(19) United States
(12) Patent Application Publication
(10) Pub. No.: US 2026/0321349 A1
(43) Pub. Date: Oct. 15, 2026

(54) AI-BASED MULTI-OMICS DATA PROCESSING FOR DETECTION OF GENOMIC INSTABILITY

(71) Applicant: OmniSeq, Inc., Buffalo, NY (US)

(72) Inventors: Zachary Daniel WALLEN, Raleigh, NC (US); Eric Allan SEVERSON, Cary, NC (US); Rebecca Ann PREVIS, Chapel Hill, NC (US); Kyle Craig STRICKLAND, Chapel Hill, NC (US); Sarabjot PABLA, Williamsville, NC (US)

(73) Assignee: OmniSeq, Inc., Buffalo, NY (US)

(21) Appl. No.: 19/222,838

(22) Filed: May 29, 2025

Related U.S. Application Data
(60) Provisional application No. 63/652,983, filed on May 29, 2024.
(60) Provisional application No. 63/765,274, filed on Feb. 28, 2025.

(51) Int. Cl. G16B 40/00; G16B 30/00; G16B 50/30

(52) U.S. Cl. CPC G16B 40/00; G16B 30/00; G16B 50/30

(57) ABSTRACT

The present disclosure relates to predicting genomic instability status in biological samples using machine learning techniques with comprehensive genomic and immune profiling (CGIP) data. Particularly, aspects are directed towards performing a genomic instability test on a biological sample. Then, multi-omics data for the subject are obtained by DNA sequencing and RNA sequencing assays, including genomic alteration data for a first set of genes and expression data for a second set of immune genes. The multi-omics data are input into a machine learning model having a tree-based architecture, which is configured to analyze features by traversing paths from root nodes to terminal nodes in each tree based on values generated from the data. The model predicts a genomic instability status, which is then provided via a user interface notification or as part of a testing report.

### CROSS-REFERENCES TO RELATED APPLICATIONS

The present application claims priority and benefit from U.S. Provisional Application No. 63/765,274, filed Feb. 28, 2025, and U.S. Provisional Application No. 63/652,983, filed May 29, 2024, the entire contents of each of which are incorporated herein by reference for all purposes.

### FIELD

The present disclosure relates to Artificial Intelligence (AI)-based techniques for processing multi-omics data to detect genomic instability in biological samples, and in particular to implementing machine learning techniques to predict microsatellite instability (MSI) status and/or homologous recombination deficiency (HRD) status using comprehensive genomic and immune profiling data.

### BACKGROUND

Genomic instability is a hallmark of cancer and is characterized by the accumulation of genetic and structural alterations that drive tumorigenesis, progression, and resistance to therapy. Two major forms of genomic instability with significant clinical relevance are homologous recombination deficiency (HRD) and microsatellite instability (MSI). These distinct forms of genomic instability represent vulnerabilities in cancer cells that can be exploited for targeted therapies, making their identification critical for advancing precision oncology.

HRD arises from the inability of cells to accurately repair double-strand DNA breaks (DSBs) via the homologous recombination (HR) repair pathway. HRD is commonly associated with mutations in key HR repair genes such as BRCA1 and BRCA2, as well as other genetic, epigenetic, or structural alterations that disrupt the HR pathway. The loss of HR repair function leads to genomic instability, manifested as chromosomal rearrangements, large-scale deletions, and other DNA aberrations. Therefore, HRD is considered a critical biomarker in cancer biology, for example, for identifying tumors likely to respond to DNA-damaging therapies such as platinum-based chemotherapies and poly(ADP-ribose) polymerase (PARP) inhibitors. These therapies induce DNA damage that HRD-positive cancer cells are unable to repair, resulting in synthetic lethality. Accordingly, there is a need for accurate identification of HRD status for guiding treatment decisions and improving outcomes for patients with HR-deficient tumors.

Traditional methods for detecting HRD rely on genetic testing for mutations in BRCA1, BRCA2, and other HR-related genes (e.g., RAD51C, RAD51D, PALB2, and ATM), as well as genomic instability scores (GIS) derived from sequencing-based analyses. However, these approaches are not only limited by high costs and the requirement for high-quality tumor samples but also lack sensitivity in detecting HRD cases that arise from non-genetic factors such as epigenetic silencing, transcriptional downregulation, or regulatory disruptions. Furthermore, traditional HRD detection methods can fail to provide a result in cases when the nature of the genomic alterations (such as highly repetitive regions or complex structural variants) interferes with accurate detection. As a result, many HRD-positive cases may go undetected using these traditional methods. Accordingly, there is a need for developing an HRD detection assay that can provide HRD status when traditional assays are unable to generate a definitive result with improved sensitivity and specificity, enabling more accurate and comprehensive patient stratification for targeted therapies.

MSI is another form of genomic instability that arises from defects in the DNA mismatch repair (MMR) system. The MMR pathway is responsible for correcting errors that occur during DNA replication, particularly in repetitive DNA sequences known as microsatellites. When the MMR system is impaired, errors accumulate in microsatellites, leading to MSI. MSI is a hallmark of certain cancers, including colorectal, endometrial, and gastric cancers, and is associated with specific genetic alterations, such as mutations in MMR genes (MLH1, MSH2, MSH6, and PMS2), as well as epigenetic silencing, such as MLH1 promoter hypermethylation. MSI status is an important biomarker with both prognostic and therapeutic implications. Tumors with high levels of MSI (MSI-high) are associated with favorable prognoses in certain cancers and exhibit increased sensitivity to immune checkpoint inhibitors, such as anti-PD-1 and anti-PD-L1 therapies. This sensitivity is thought to result from the high mutational burden in MSI-high tumors, which leads to the generation of neoantigens that can elicit robust immune responses.

Traditional methods for detecting MSI include polymerase chain reaction (PCR)-based assays to analyze microsatellite loci and immunohistochemistry (IHC) to assess the expression of MMR proteins. These methods can be time-consuming, expensive, and reliant on high-quality tissue samples. More recently, NGS-based approaches have been developed to evaluate MSI status. However, these existing MSI detection methods are particularly susceptible to test failure due to the repetitive nature of microsatellites and other technical limitations. In such cases, a conclusive MSI status cannot be determined, which may delay or preclude optimal patient management. Consequently, there is also a need for accurate and reliable determination of MSI status for identifying patients who are likely to benefit from immunotherapy.

### BRIEF SUMMARY

In various embodiments, a computer-implemented method is provided, comprising performing a genomic instability testing on a first sample obtained from a subject to generate an indication of a genomic instability of the first sample, wherein the indication comprises a presence of the genomic instability, an absence of the genomic instability, or a failure to make the indication; performing an AI-based genomic instability determination by: obtaining, using one or more multi-analyte assays, multi-omics data for the subject, wherein the one or more multi-analyte assays comprise DNA sequencing and RNA sequencing, and wherein the multi-omics data comprises genomic alteration data for a first set of genes obtained using the DNA sequencing and expression data for a second set of immune genes obtained using the RNA sequencing; inputting the multi-omics data into a machine learning model, wherein the machine learning model comprises a tree-based architecture configured to analyze one or more features by traversing a path from a root node to a terminal node in each tree of the tree-based architecture based on values of one or more features generated from the multi-omics data; and predicting a genomic instability status of the subject using the machine learning model based on the paths and/or the terminal nodes; and providing the predicted genomic instability status via a notification to a user interface or through a testing report.

In some embodiments, in response to the failure to make the indication, providing the indication through the testing report; and in response to the indication of the presence or absence of the genomic instability, comparing the indication with the predicted genomic instability status, and providing the comparison through the testing report confirming if the indication is consistent or inconsistent with the predicted genomic instability status.

In some embodiments, the genomic instability is microsatellite instability (MSI), and the predicted genomic instability status is an MSI status.

In some embodiments, the genomic instability is homologous recombination deficiency (HRD), and the predicted genomic instability status is an HRD status.

In some embodiments, the MSI status is MSI-high or microsatellite stable (MSS).

In some embodiments, the HRD status is HRD-high or HRD-low.

In some embodiments, the multi-omics data is obtained by obtaining a biopsy sample from the subject, wherein the biopsy sample is the first sample or a portion thereof, or a different sample; extracting DNA and RNA from the biopsy sample; performing the DNA sequencing to obtain DNA sequencing data; performing the RNA sequencing to obtain RNA sequencing data; generating the genomic alteration data for the first set of genes based on the DNA sequencing data; and generating the expression data for the second set of immune genes based on the RNA sequencing data.

In some embodiments, the computer-implemented method further comprises imputing missing genomic alteration data for one or more genes in the first set of genes or missing expression data for one or more immune genes in the second set of immune genes based on the DNA sequencing data, the RNA sequencing data, or available genomic alteration data or available expression data from one or more other genes in the rest of the first set of genes or the second set of immune genes.

In some embodiments, the imputing is performed using a Multivariate Imputation by Chained Equations (MICE) method with predictive mean matching or by fitting a linear regression model.

In some embodiments, the computer-implemented method further comprises performing a confirmatory testing using a second sample obtained from a subject to confirm the predicted genomic instability status, wherein the second sample is the first sample or a portion thereof, or a different sample.

In some embodiments, the confirmatory testing comprises one or more of: (a) MLH1 promoter methylation analysis, (b) mismatch repair gene sequencing, (c) loss of heterozygosity (LOH) analysis, (d) functional DNA repair assay, (e) fluorescence in situ hybridization (FISH) assay, (f) chromosomal microarray analysis, (g) targeted sequencing using an extended genomic instability panel, (h) Sanger sequencing, and (i) immunohistochemistry for mismatch repair proteins.

In some embodiments, the computer-implemented method further comprises outputting the testing report, wherein the testing report comprises the indication for the genomic instability testing, the predicted genomic instability status using the AI-based genomic instability determination, the multi-omics data and/or features used by the machine learning model to determine the predicted genomic instability status.

In some embodiments, the first set of genes comprises at least 10 genes, and/or the second set of immune genes comprises at least 20, 30, 40, 50, or 60 genes.

In some embodiments, the genomic alteration data comprises data of single nucleotide variants (SNVs), insertions and deletions (indels), copy number variations (CNVs), gene fusions, splice variants, and/or tumor mutational burden (TMB).

In some embodiments, the multi-omics data for the subject further comprises immunohistochemistry data, cell proliferation data, tumor inflammation data, and/or cancer testis antigen burden data.

In some embodiments, (i) the immunohistochemistry data comprises PD-L1 immunohistochemistry data, (ii) the cell proliferation data comprises Ki-67 proliferation index data, (iii) the tumor inflammation data comprises gene expression signatures associated with tumor inflammation, and/or (iv) the cancer testis antigen burden data comprises expression levels of one or more cancer testis antigens.

In some embodiments, the computer-implemented method further comprises (i) staining a formalin-fixed, paraffin-embedded (FFPE) tissue sample obtained from the subject with an antibody specific to a protein of interest and evaluating the stained FFPE tissue sample by light microscopy or digital image analysis to obtain the immunohistochemistry data, the cell proliferation data, the tumor inflammation data, and/or the cancer testis antigen burden data; and/or (ii) extracting RNA from the FFPE tissue sample and performing gene expression profiling RNA sequencing or quantitative PCR to obtain the cell proliferation data, the tumor inflammation data, and/or the cancer testis antigen burden data.

In some embodiments, the machine learning model is trained using training samples with known genomic instability statuses to learn patterns from multi-omics data of the training samples to predict genomic instability statuses of training samples.

In some embodiments, the computer-implemented method further comprises training the machine learning model, wherein the training comprises: obtaining training data associated with the training samples, wherein the training data comprises genomic alteration data, gene expression data, and the known genomic instability statuses; selecting, using a feature-selection model, candidate features from a set of features based on the training data, wherein the candidate features are predictors to the genomic instability status; training the machine learning model using input data with the candidate features to predict the genomic instability status; and outputting the trained machine learning model.

In some embodiments, the computer-implemented method further comprises dividing the input data into training sets and testing sets; performing the training using the training sets to learn a mapping from the candidate features to the genomic instability status; evaluating the trained machine learning model on the testing sets to generate one or more performance metrics indicative of an ability of the trained machine learning model to predict the genomic instability status; and outputting the trained machine learning model with the one or more performance metrics.

In some embodiments, the one or more performance metrics comprise sensitivity, specificity, positive predictive value (PPV), negative predictive value (NPV), balanced accuracy, F1 score, and/or Receiver Operating Characteristic-Area Under the Curve (ROC-AUC) value.

In some embodiments, the feature-selection model is a Boruta algorithm-based model or a random forest model.

In some embodiments, the training samples are biological samples obtained from patients having been diagnosed with cancer.

In some embodiments, the cancer is one or more cancer selected from the group consisting of: colorectal cancer, colon adenocarcinoma, rectum adenocarcinoma, uterine cancer, adrenal gland cancer, bile duct cancer, bladder cancer, bone cancer, bone marrow and blood cancer, brain cancer, breast cancer, cervix cancer, esophageal cancer, eye cancer, head and neck cancer, kidney cancer, liver cancer, lung cancer, lymph node cancer, nervous system cancer, ovarian cancer, pancreatic cancer, pleura cancer, prostate cancer, skin cancer, soft tissue cancer, stomach cancer, testicular cancer, thymus cancer, thyroid cancer, and uterine cancer.

In some embodiments, the computer-implemented method further comprises selecting a plurality of machine learning algorithms; training each machine learning model with one or more machine learning algorithm in the plurality of machine learning algorithms; generating a set of performance metrics for each trained machine learning model; and selecting the machine learning model based on the set of performance metrices.

In some embodiments, the plurality of machine learning algorithms comprises Classification and Regression Trees (CART), stochastic gradient boosting, Gradient Boosting Machine (GBM), K-Nearest Neighbors (K-NN), Linear Discriminant Analysis (LDA), Logistic Regression, Multi-Layer Perceptron (MLP), Naïve Bayes, Random Forest, Support Vector Machine with Radial Basis Function Kernel (SVM Radial), and/or Extreme Gradient Boosting (XGBoost).

In some embodiments, the set of performance metrics comprises sensitivity, specificity, positive predictive value (PPV), negative predictive value (NPV), balanced accuracy, F1 score, and/or Receiver Operating Characteristic-Area Under the Curve (ROC-AUC) value.

In some embodiments, the one or more performance metrics or the set of performance metrics are generated using a k-fold cross-validation.

In various embodiments, a computer-implemented method is provided for training a machine learning model to be used for an AI-based genomic instability determination, comprising: obtaining training data associated with training samples, wherein the training data comprises genomic alteration data, gene expression data, and known genomic instability statuses; selecting, using a feature-selection model, candidate features from a set of features based on the training data, wherein the candidate features are predictors to the genomic instability status; training the machine learning model using input data with the candidate features to predict the genomic instability status; and outputting the trained machine learning model.

In some embodiments, a non-transitory computer-readable medium is provided that stores instructions which, when executed by one or more processors, cause the one or more processors to perform operations in any of the computer implemented methods disclosed herein.

In some embodiments, a system is provided that includes one or more processors, and one or more computer-readable media storing instructions which, when executed by the one or more processors, cause the system to perform operations in any of the computer implemented methods disclosed herein.

In some embodiments, a computer-program product is provided that is tangibly embodied in a non-transitory computer-readable memory that includes instructions which, when executed by one or more processors, cause the one or more processors to perform operations in any of the computer implemented methods disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the disclosure claimed. Thus, it should be understood that although the present disclosure has been specifically disclosed by embodiments and optional features, modification and variation of the concepts herein disclosed may be resorted to by those skilled in the art, and that such modifications and variations are considered to be within the scope of this application as defined by the appended claims.

### BRIEF DESCRIPTION OF THE DRAWINGS

Aspects and features of the various embodiments will be more apparent by describing examples with reference to the accompanying drawings, in which:

FIG. 1 shows an exemplary computing environment for implementing genomic instability prediction workflows in accordance with various embodiments.

FIG. 2 shows an exemplary workflow for performing a genomic instability prediction assay that enables the prediction of genomic instability status for a biological sample in accordance with various embodiments.

FIG. 3 shows an exemplary workflow for training a machine learning model to genomic instability status using multi-omics data in accordance with embodiments.

FIG. 4 shows an exemplary system for generating pathology images and sequencing data for comprehensive genomic and immune profiling in accordance with various embodiments.

FIG. 5 shows feature importance scores for genomic and gene expression (GEx) factors using a Boruta algorithm in accordance with various embodiments.

FIG. 6 shows performance of various ML algorithms across six metrics in accordance with various embodiments.

FIG. 7 shows a block diagram of a machine learning pipeline to train, validate, and implement one or more machine learning models to predict genomic instability status in accordance with various embodiments.

FIG. 8 shows an exemplary illustration of a gradient boosting decision tree machine learning model in accordance with various embodiments.

FIG. 9 provides an exemplary overview of the OmniSeq INSIGHT assay in accordance with various embodiments.

FIG. 10 shows a photomicrograph of a case identified as potentially MSI-H that failed MSI testing by NGS in accordance with various embodiments.

FIG. 11 provides a workflow summary for calculating gene expression ranks in accordance with various embodiments.

FIG. 12 shows the features chosen during feature selection as important for distinguishing between MSI-High versus MSS cases in accordance with various embodiments.

FIG. 13 provides the workflow for generation and testing of the classification models in accordance with various embodiments. (A) Obtained NGS data from OmniSeq® INSIGHT (OSI), including genomic variant detection within 523 genes (SNVs, insertions, deletions) and RNA sequencing of 395 immune-related genes. (B) Immune gene expression and genomic variant data were collected for 2,282 CRC cases that were evaluated as part of routine clinical practice and passed microsatellite testing. (C) Feature selection was performed to identify important gene expression and genomic features differentiating MSI from MSS cases. (D) CRC cases were divided into training and testing cohorts using a 70/30 split and multiple classification models were trained, tested, and evaluated using features identified in feature selection and TMB. (E) Model performance was validated on orthogonal datasets, including TCGA COAD/READ cases (independent cohort) and UC OSI cases (separate anatomic location). (F) Top performing model was used to predict MSI status in CRC cases failing microsatellite testing to assess the feasibility of predicting MSI for failed cases.

FIG. 14 provides a workflow for predicting MSI status in cases failing microsatellite testing in accordance with various embodiments. (A) Clinical NGS testing performed per protocol using OmniSeq INSIGHT (OSI). (B) Cases failing microsatellite testing identified and data for model features (TMB, gene expression rank for 62 genes, gene-level summarized SNV presence or absence for 76 genes) extracted from their routine OSI testing. (C) Model feature data combined with cohort of cases passing microsatellite testing and missing datapoints imputed using MICE method (gene expression ranks, SNVs) or fitted linear regression model (TMB). (D) MSI status prediction using trained classification model. (E) Binary output of “likely MSI-high” or “likely MSS” received from classification model. (F) Addition of classification model prediction on the OSI reports along with language suggesting further testing for MSI if case received a “likely MSI-high” classification.

FIG. 15 is a bar graph showing model performance metrics when evaluating trained classification models in the CRC testing cohort in accordance with various embodiments.

FIG. 16 is a bar graph showing model performance metrics when evaluating trained classification models in the TCGA COAD/READ cohort in accordance with various embodiments.

FIG. 17 is a bar graph showing model performance metrics when evaluating random forest (rf), classification and regression trees (rpart), and stochastic gradient boosting (gbm) models in the UC cohort in accordance with various embodiments.

FIG. 18A shows area under the receiver operating curve (AUC-ROC) and FIG. 18B shows a graph for precision-recall (AUC-PR) for each cohort using the classification and regression tree model, in accordance with various embodiments.

### TERMS

As used herein, the articles “a” and “an” are used herein to refer to one or to more than one (i.e. at least one) of the grammatical object of the article. By way of example, an element means at least one element and can include more than one element.

As used herein, the terms “about,” “approximately,” and “substantially” are used interchangeably and mean within an acceptable error range for the particular value as determined by one of ordinary skill in the art, and thus depend in part on how the value is measured or determined, e.g., the limitations of the measurement system. For example, the term “substantially,” “approximately,” or “about” may be substituted with “within [a percentage] of” what is specified, where the percentage includes 0.1, 1, 5, and 10 percent. Where particular values are described in the application and claims, unless otherwise stated, the term “about” means within an acceptable error range for the particular value.

As used herein, the term “antibody” refers to an immunoglobulin (Ig) molecule, an antigen binding fragment thereof or a binding derivative thereof. An antigen binding fragment of an antibody contains an antigen binding site that specifically binds an antigen. The antibodies (Abs) may be monoclonal antibodies, polyclonal antibodies, or multi-specific antibodies (e.g., bispecific antibodies). Examples of antibodies include immunoglobulin (Ig) types IgG, IgD, IgE, IgA and IgM. The antibodies may be native antibodies or recombinant antibodies. The antibodies may be produced by host cells. The term antibody is not restricted to immunoglobulins derived from any particular mammalian species and includes murine, human, equine, and camelids antibodies (e.g., human antibodies). The term “antibody” encompasses antibodies isolatable from natural sources or from animals following immunization with an antigen as well as engineered antibodies including monoclonal antibodies, bispecific antibodies, tri-specific, chimeric antibodies, humanized antibodies, human antibodies, CDR-grafted, veneered, or deimmunized (e.g., to remove T-cell epitopes) antibodies.

As used herein, when an action is “based on” something, this means the action can be based at least in part on at least a part of the something.

As used herein, the term “cancer” refers to an abnormal state or condition characterized by rapidly proliferating cell growth. Rapidly proliferating cells may be categorized as pathologic (i.e., characterizing or constituting a disease state), or may be categorized as non-pathologic (i.e., a deviation from normal but not associated with a disease state). In addition, cancer cells can spread locally or through the bloodstream and lymphatic system to other parts of the body. In general, a cancer will be associated with the presence of one or more tumors (i.e., abnormal cell masses). The term “tumor” is meant to include all types of cancerous growths or oncogenic processes, metastatic tissues or malignantly transformed cells, tissues, or organs, irrespective of histopathologic type or stage of invasiveness. Examples of cancer include malignancies of various organ systems, such as bladder cancers, lung cancers, breast cancers, thyroid cancers, lymphoid cancers, gastrointestinal cancers, and genito-urinary tract cancers. Cancer can also refer to adenocarcinomas, which include malignancies such as colon cancers, renal-cell carcinoma, prostate cancer and/or testicular tumors, non-small cell carcinoma of the lung, cancer of the small intestine, and cancer of the esophagus. Carcinomas are malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. An “adenocarcinoma” refers to a carcinoma derived from glandular tissue or in which the tumor cells form recognizable glandular structures. “Melanoma” refers to a tumor arising from a melanocyte. Melanomas occur most commonly in the skin and are frequently observed to metastasize widely. In various embodiments, the patients have colorectal (CRC) and/or endometrial (EMCA) solid tumors.

As used herein, the term “multi-omics data” refers to integrated datasets that combine multiple layers of biological information derived from various omics fields, including, but not limited to, genomics, transcriptomics, epigenomics, proteomics, metabolomics, lipidomics, and immunomics. Unless specifically limited, the term encompasses data obtained from high-throughput technologies that analyze DNA, RNA, proteins, metabolites, lipids, or immune system components. Multi-omics data may include information on genetic variations, gene expression levels, epigenetic modifications, protein abundance, metabolic profiles, and immune signatures. Such data can be derived from naturally occurring biological samples, synthetic systems, or computational simulations. Multi-omics data also includes all forms of integrative analyses, such as cross-layer correlations, pathway modeling, and systems-level interactions.

As used herein, the term “nucleic acid” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar properties as the reference nucleic acid. A nucleic acid sequence can comprise combinations of deoxyribonucleic acids and ribonucleic acids. Such deoxyribonucleic acids and ribonucleic acids include both naturally occurring molecules and synthetic analogues. Nucleic acids also encompass all forms of sequences including, but not limited to, single-stranded forms, double-stranded forms, hairpins, stem-and-loop structures, and the like.

As described herein, the terms “non-tumor tissue/cell(s)” and “non-cancerous tissue/cell(s)” are used interchangeably and refer to tissues or cells that are not tumor or cancerous. Examples of non-tumor tissues/cells include, but are not limited to, pathologically healthy/normal cells, immune cells, non-malignant cells, and cells/tissues displaying abnormal pathology that is not related to cancer (e.g., inflammation, increase inflammatory cells, increase in apoptotic cells, and the like).

As described herein, “patient,” and “subject” are used interchangeably and refer to any single animal, more preferably a mammal (e.g., humans). In certain embodiments, subjects are “patients,” (i.e., living humans) that are receiving medical care for a disease or condition. This includes persons with no defined illness who are being investigated for signs of pathology. In some embodiments, the patient or subject may be at risk of/diagnosed with/being treated for cancer. For example, the patient or subject may be at risk of developing cancer, diagnosed with cancer, and/or is being treated for cancer.

As used herein, the term “sample,” “biological sample,” “patient sample,” “tissue,” and “tissue sample” refer to any sample including a biomolecule (such as a protein, a peptide, a nucleic acid, a lipid, a carbohydrate, or a combination thereof) that is obtained from any organism including viruses, and the terms may be used interchangeably. Other examples of organisms include mammals (such as humans; veterinary animals like cats, dogs, horses, cattle, and swine; and laboratory animals like mice, rats and primates), insects, annelids, arachnids, marsupials, reptiles, amphibians, bacteria, and fungi. Biological samples include tissue samples (such as tissue sections and needle biopsies of tissue), cell samples (such as cytological smears such as Pap smears or blood smears or samples of cells obtained by microdissection), or cell fractions, fragments or organelles (such as obtained by lysing cells and separating their components by centrifugation or otherwise). Other examples of biological samples include blood, serum, urine, semen, fecal matter, cerebrospinal fluid, interstitial fluid, mucous, tears, sweat, pus, biopsied tissue (for example, obtained by a surgical biopsy or a needle biopsy), nipple aspirates, cerumen, milk, vaginal fluid, saliva, swabs (such as buccal swabs), or any material containing biomolecules that is derived from a first biological sample. In certain embodiments, the term “biological sample” as used herein refers to a sample (such as a homogenized or liquefied sample) prepared from a tumor or a portion thereof obtained from a subject.

The use herein of the terms including, comprising, or having, and variations thereof, is meant to encompass the elements listed thereafter and equivalents thereof as well as additional elements. Embodiments recited as including, comprising, or having certain elements are also contemplated as consisting essentially of and consisting of those certain elements. As used herein, the term “and/or” refers to and encompasses any and all possible combinations of one or more of the associated listed items, as well as the lack of combinations were interpreted in the alternative (or).

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values (e.g., integer, whole number, decimal, fraction, and the like) between and including the lowest value and the highest value enumerated are to be considered to be expressly stated in this disclosure.

Unless defined otherwise, all technical and scientific terms used herein have the same meaning as commonly understood by one of ordinary skill in the art to which this disclosure belongs. Although any methods and materials similar to or equivalent to those described herein can be used in the practice or testing of the application, the preferred methods and materials are now described.

### DETAILED DESCRIPTION

The ensuing description provides preferred exemplary embodiments only, and is not intended to limit the scope, applicability or configuration of the disclosure. Rather, the ensuing description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the following description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

Also, it is noted that individual embodiments may be described as a process which is depicted as a flowchart, a flow diagram, a data flow diagram, a structure diagram, or a block diagram. Although a flowchart or diagram may describe the operations as a sequential process, many of the operations may be performed in parallel or concurrently. In addition, the order of the operations may be re-arranged. A process is terminated when its operations are completed, but could have additional steps not included in a figure. A process may correspond to a method, a function, a procedure, a subroutine, a subprogram, etc. When a process corresponds to a function, its termination may correspond to a return of the function to the calling function or the main function.

Publications cited herein and the material for which they are cited are hereby specifically incorporated by reference in their entireties.

### I. INTRODUCTION

Genomic instability, characterized by a high frequency of mutations, chromosomal rearrangements, and structural alterations in DNA, is a defining hallmark of cancer. It underpins tumor evolution by disrupting normal cellular processes, promoting genetic diversity, and fostering resistance to therapy. Detecting genomic instability can be critical for cancer diagnosis, prognosis, and treatment selection, as different forms of instability can reveal unique vulnerabilities in tumor cells that can be therapeutically targeted. Among the various manifestations of genomic instability, homologous recombination deficiency (HRD) and microsatellite instability (MSI) represent two notable examples that have significant clinical implications. HRD results from the failure of the homologous recombination (HR) DNA repair pathway, responsible for repairing double-strand breaks with high fidelity, leading to the accumulation of large-scale genomic alterations. MSI arises from defects in the DNA mismatch repair (MMR) pathway, resulting in length alterations in microsatellite regions of the genome. Both deficiencies create unique vulnerabilities in tumor cells that can be therapeutically exploited.

Comprehensive genomic profiling (CGP) is a next generation sequencing (NGS) approach that detects and characterizes genomic alterations across hundreds of genes. The types of genomic alterations and signatures that are typically identified in CGP include small DNA variants (e.g., single nucleotide variants (SNVs) and small insertions or deletions (indels)), copy number variations (CNVs), gene fusions, splice variants, tumor mutational burden, and microsatellite instability, making CGP an incredibly valuable tool in cancer diagnostics. CGP may be performed on solid tumor tissue, formalin-fixed, paraffin-embedded (FFPE) tumor tissue, or liquid biopsies and provides insight about a cancer's genomic makeup allowing clinicians to make informed decisions about a patient's treatment.

NGS (also known as massively parallel sequencing) technologies allow for millions of bases to be concurrently sequenced. Briefly, NGS uses a process known as clonal amplification to amplify the DNA fragments of a patient sample and bind them to a flow cell. Then, a sequencing by synthesis method is used where fluorescently labeled nucleotides compete for addition onto a growing chain based on the sequence of the template. A light source is used to excite the unique fluorescence signal associated with each nucleotide and the emission wavelength and fluorescence signal intensity determine the base call. Each lane in a flow cell can hold hundreds to millions of DNA templates, giving NGS its massively parallel sequencing capabilities. Importantly, NGS technologies have greatly improved the flexibility of genetic screenings, providing highly sensitive and accurate high-throughput platforms for large-scale genomic testing, including sequencing of single genes, targeted gene panels, whole-exomes, and whole-genomes.

Despite these advances, there are persistent technical and biological challenges in detecting genomic instability using conventional and next generation sequencing-based methods. For example, HRD is commonly assessed by calculating scores based on large-scale chromosomal events, such as loss of heterozygosity, telomeric allelic imbalance, and large-scale state transitions. These approaches may require matched tumor-normal specimens and sophisticated data analysis pipelines, which are not always available in clinical settings. Furthermore, these methods may not capture all mechanisms underlying HRD, such as epigenetic silencing or transcriptional downregulation of key genes.

Similarly, detecting MSI presents unique technical hurdles. Microsatellites are short, repetitive DNA sequences distributed throughout the genome. The repetitive nature of these regions makes them difficult for PCR-based and sequencing-based methods to accurately analyze, especially when DNA is fragmented or degraded, as is common in formalin-fixed, paraffin-embedded samples. Sequencing errors, slippage during amplification, and high mutation rates complicate both the alignment and interpretation of these regions. As a result, MSI testing can fail to provide a conclusive result for a significant number of samples, necessitating retesting or alternative approaches.

The inability to obtain a reliable genomic instability determination from standard assays can delay or hinder patient care. Failures may be due to poor DNA quality, insufficient sample quantity, or intrinsic technical limitations of the assay, such as inability to confidently sequence repetitive regions or detect certain types of structural variants. When a definitive result cannot be obtained, patients may lose access to precision therapies or clinical trials specifically designed for tumors with genomic instability.

To address these limitations and challenges, techniques disclosed herein provide robust and scalable approaches that can accurately predict genomic instability status even when conventional testing fails or is inconclusive. Integrating multi-omics data, such as genomic alterations and gene expression profiles, offers a broader perspective on the molecular state of a tumor. Machine learning models are particularly effective for this purpose, as they can learn complex patterns and relationships from large, multi-dimensional datasets. These models are capable of predicting genomic instability status by analyzing diverse data types, including those generated by multi-analyte assays. Importantly, such approaches can provide a result when traditional laboratory tests are unsuccessful, thereby supporting clinical decision-making and precision oncology.

As an illustrative example, the techniques disclosed herein include computer-implemented methods, systems, and computer program products that employ machine learning models to predict genomic instability status using multi-omics data. For example, when a genomic instability testing report from a first sample yields a failure or inconclusive result, multi-omics data is then obtained for the subject through multi-analyte assays. This multi-omics data includes genomic alteration data for a first set of genes and expression data for a second set of immune genes. The multi-omics data is then input into a machine learning model (e.g., a random forest model) that has been trained on samples with known genomic instability status to learn predictive patterns across the multi-omics features. The model generates a prediction of the subject's genomic instability status, and this result is provided via a user interface or included in an updated testing report. When the genomic instability testing report from the first sample yields a conclusive result, the AI-based approach can be also used as a confirmatory testing. to further improve the overall testing accuracy.

The integration of AI-based models into the computing system enhances its structural efficiency and computational performance by optimizing the allocation and utilization of processing resources during genomic instability analysis. Unlike conventional techniques, which often operate on static algorithms with limited adaptability, AI-based models dynamically adjust computational workflows based on the complexity of the input data, ensuring optimal resource usage. In particular, the machine learning model can be implemented as a decision tree model, such as a random forest model or a model generated using recursive partitioning (e.g., an rpart model). Decision tree-based models are inherently memory-efficient because they store the decision rules and splits in a compact tree structure, rather than relying on large numbers of coefficients or complex matrix operations. This structure allows the model to make predictions by traversing the tree with simple, sequential decisions, significantly reducing memory requirements and computational overhead during both training and inference. For random forest models, which aggregate the output of multiple decision trees, the ensemble can still be stored and executed efficiently, as each tree remains a lightweight and interpretable object. These models leverage advanced machine learning architectures and parallel computing capabilities, enabling distributed processing across multiple nodes or GPUs, which significantly reduces latency and computation times. Additionally, the AI-based models incorporate advanced data preprocessing and feature extraction techniques, which streamline the computational pipeline by filtering out irrelevant data and focusing on high-impact variables. This also results in a reduction in memory overhead and computational redundancy. By using decision tree-based models, the disclosed techniques are able to efficiently handle increasingly large and complex datasets without sacrificing accuracy or scalability, thereby surpassing the limitations of traditional methods in genomic analysis.

In addition to improving computational efficiency, the disclosed techniques provide broad adaptability for genomic instability detection by allowing the selection and integration of diverse omic input data, such as gene expression profiling, genomic alterations, and immune-related features. This flexibility ensures that the system can be configured for the detection of any particular type of genomic instability, depending on the clinical application or research need. For example, gene expression profiling can be used to reveal the functional consequences of homologous recombination pathway disruption, supporting the detection of HRD status. Similarly, the integration of immune markers, mutational burden, and gene-level alterations can support the detection of MSI status or other instability phenotypes. Machine learning models, including decision tree-based and ensemble models such as random forests, can process large and complex transcriptomic and genomic datasets to identify predictive signatures associated with HRD, MSI, or any other form of genomic instability. The input features for prediction are not fixed but can be tailored to include gene expression changes, mutation status, immune cell infiltration, or other relevant biomarkers, depending on the specific type of genomic instability being assessed and the available data. Unlike traditional methods that may be limited to a single assay type or require matched tumor-normal specimens, these techniques can flexibly accommodate high-quality data from a variety of sources, including FFPE tissue or liquid biopsy. AI-based models further improve the sensitivity and specificity of genomic instability prediction by detecting complex, multidimensional patterns in the data that may be overlooked by conventional approaches. This adaptability enables robust and accurate detection of any clinically relevant genomic instability, facilitating precision oncology and more personalized therapeutic strategies across a broad range of cancer types and testing scenarios.

### II. EXEMPLARY COMPUTING ENVIRONMENTS

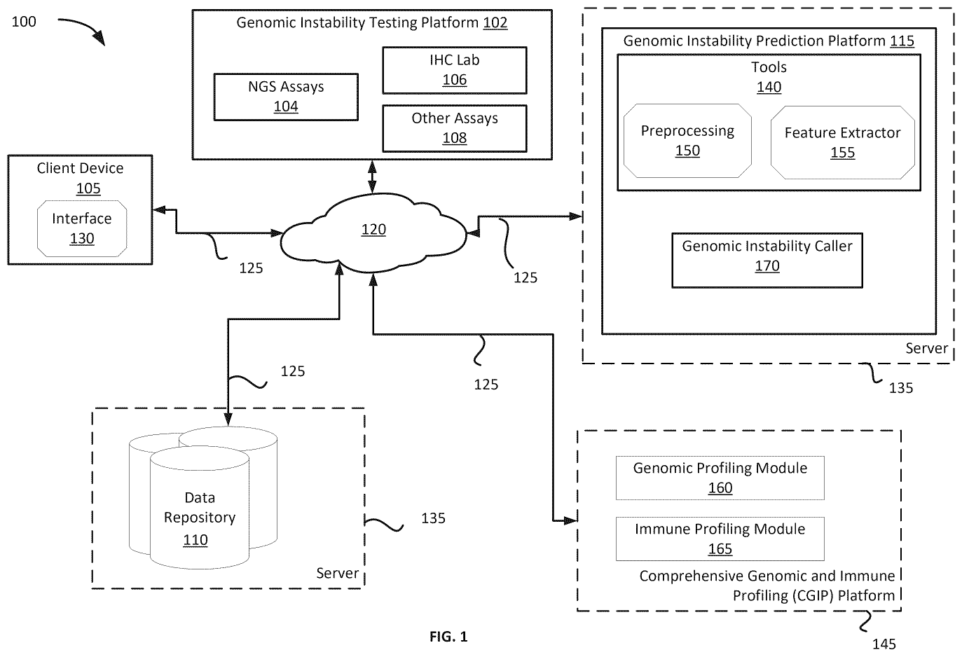

FIG. 1 shows an exemplary computing environment 100 for implementing genomic instability prediction workflows in accordance with various embodiments. The computing environment 100 includes a genomic instability testing platform 102, a client device 105, a server 135, a comprehensive genomic and immune profiling (CGIP) platform 145, and a network 120 connecting to components of the computing environment 100. Although FIG. 1 illustrates a particular arrangement of the genomic instability testing platform 102, the client device 105, the server 135, the CGIP platform 145, and the network 120, this disclosure contemplates any suitable arrangement of these components and additional components. As an example, and not by way of limitation, two or more client devices 105, the server 135, and the CGIP platform 145 may be connected to each other directly, bypassing the network 120. As another example, two or more client devices 105, the server 135, and the CGIP platform 145 may be physically or logically co-located with each other in whole or in part. Moreover, although FIG. 1 illustrates a particular number of the components, this disclosure contemplates any suitable number of components (e.g., client devices 105, servers 135, CGIP platforms 145, and networks 120). As an example, and not by way of limitation, computing environment 100 may include multiple client devices 105, multiple servers 135, multiple CGIP platforms 145, and multiple networks 120.

This disclosure contemplates any type of network 120 familiar to those skilled in the art that may support data communications using any of a variety of available protocols including without limitation TCP/IP (transmission control protocol/Internet protocol), SNA (systems network architecture), IPX (Internet packet exchange), AppleTalk®, and the like. Merely by way of example, network(s) 120 may be a local area network (LAN), networks based on Ethernet, Token-Ring, a wide-area network (WAN), the Internet, a virtual network, a virtual private network (VPN), an intranet, an extranet, a public switched telephone network (PSTN), an infra-red network, a wireless network (e.g., a network operating under any of the Institute of Electrical and Electronics (IEEE) 102.11 suite of protocols, Bluetooth®, and/or any other wireless protocol), and/or any combination of these and/or other networks.

Links 125 may connect a client device 105, a server 135 or a unit thereof (e.g., a data repository 110, a genomic instability prediction platform 115), or a CGIP platform 145 or a unit thereof (e.g., a genomic profiling module 160, or an immune profiling module 165) to a network 120 or to each other. This disclosure contemplates any suitable links 125. In particular embodiments, one or more links 125 include one or more wireline (such as for example Digital Subscriber Line (DSL) or Data Over Cable Service Interface Specification (DOCSIS)), wireless (such as for example Wi-Fi or Worldwide Interoperability for Microwave Access (WiMAX)), or optical (such as for example Synchronous Optical Network (SONET) or Synchronous Digital Hierarchy (SDH)) links. In particular embodiments, one or more links 125 each include an ad hoc network, an intranet, an extranet, a VPN, a LAN, a WLAN, a WAN, a WWAN, a MAN, a portion of the Internet, a portion of the PSTN, a cellular technology-based network, a satellite communications technology-based network, another link 125, or a combination of two or more such links 125. Links 125 need not necessarily be the same throughout the computing environment 100. One or more first links 125 may differ in one or more respects from one or more second links 125.

The genomic instability testing platform 102 serves as a component for performing genomic instability testing on biological samples. The genomic instability testing platform 102 may comprise several specialized modules, including NGS assays 104, an immunohistochemistry laboratory (IHC lab) 106, and other assays 108. Each of these modules is capable of generating specific omic data for characterizing genomic instability in clinical and research settings.

For example, the NGS assays 104 is configured to perform next generation sequencing on patient samples. The NGS assays 104 typically includes automated systems for DNA and RNA extraction, high-throughput sequencing instruments, and robotic equipment for sample preparation. The NGS assays 104 is further supported by specialized software for sequence alignment, variant calling, and data analysis, which can operate on local servers (e.g., the server 135) or through cloud-based infrastructure (e.g., the network 120), ensuring efficient processing of large datasets. DNA sequencing conducted within the NGS assays 104 enables comprehensive detection of genomic alterations, including single nucleotide variants, small insertions or deletions, copy number variations, and larger structural changes across numerous genes relevant to genomic instability. This DNA-based analysis also facilitates the identification of key genomic signatures (e.g., MSI, HRD, or other genomic instability). In addition, RNA sequencing may be performed to analyze gene expression profiles from the same or related patient samples. Through RNA sequencing, the NGS assays 104 can quantify immune gene expression, detect gene fusions, and identify aberrant splicing events, all of which may contribute to or indicate genomic instability.

In some embodiments, the sequencing data produced by the NGS assays 104 may also be used to inform or trigger confirmatory testing workflows (including the AI-based genomic instability prediction using the genomic instability prediction platform 115) when initial genomic instability results are ambiguous or when further validation is clinically indicated. For example, if the primary NGS analysis suggests a high probability of MSI or HRD but the evidence is borderline or inconclusive, additional confirmatory tests can be ordered. These may include sequencing of specific mismatch repair genes, MLH1 promoter methylation analysis, loss of heterozygosity (LOH) studies, or targeted Sanger sequencing to validate potentially pathogenic variants. The confirmatory testing processes may be coordinated with other modules within the genomic instability testing platform 102, such as the IHC laboratory 106 or other assays 108. The confirmatory testing may also include the AI-based genomic instability prediction. In other instances, when the primary NGS analysis indicates a failure to provide MSI or HRD determination, the sequencing data may be sent to the genomic instability prediction platform 115 via the network 120 to perform the AI-based genomic instability prediction.

The IHC lab 106 is configured to perform IHC-based assays on patient samples, such as tissue specimens obtained from biopsies or surgical resections. This lab includes equipment for preparing and processing FFPE tissue sections, automated or manual stainers for applying antibodies, and microscopes or digital slide scanners for visualizing stained slides. In the IHC lab 106, specific protein markers such as the mismatch repair proteins MLH1, MSH2, MSH6, and PMS2 can be detected by applying targeted antibodies and assessing their presence or absence in tumor tissue. The evaluation of these markers is often used for conventional detection of MSI status, as the loss of expression of one or more mismatch repair proteins can be highly indicative of underlying genomic instability. Beyond MSI, the IHC lab 106 can also be used to detect other protein-level changes relevant to cancer biology, including markers of cell proliferation, immune cell infiltration, or expression of cancer testis antigens. The data generated in the IHC lab 106 provides another layer of biological information that can complement findings from NGS assays 104. For example, IHC results may validate or clarify ambiguous NGS findings, offer orthogonal evidence of genomic instability, or help resolve cases where sequencing-based tests yield inconclusive results. In some embodiments, data generated at the IHC lab 106 may be sent to the genomic instability prediction platform 115 to facilitate the genomic instability prediction or assessment.

The other assays 108 encompasses a variety of additional genomic instability testing assays that may be employed for comprehensive genomic instability detection. For example, the other assays 108 may include PCR-based fragment analysis designed to evaluate microsatellite loci for length variability for MSI status determination. Through capillary electrophoresis or similar detection systems, these assays can compare the size of microsatellite repeats in tumor and normal DNA, providing direct or indirect evidence of instability. The other assays 108 module may also incorporate methylation-specific assays, such as those targeting promoter regions like MLH1, to identify epigenetic silencing events that are frequently associated with MSI in certain cancer types. Additionally, functional assays assessing DNA repair capacity may be included in the other assays 108. These can involve cell-based assays or biochemical tests that evaluate the proficiency of HR or other repair pathways, offering direct evidence of HRD or other forms of defective DNA repair. Equipment used in the other assays 108 may comprise PCR machines, capillary electrophoresis instruments, quantitative methylation-specific PCR platforms, and systems for performing functional DNA repair assays, as well as associated reagents and quality control tools.

The inclusion of other assays 108 ensures that the genomic instability testing platform 102 can flexibly address a wide range of clinical scenarios and sample types. For example, when NGS assays 104 or the IHC lab 106 yield inconclusive or ambiguous results, the other assays 108 provides alternative or confirmatory testing options to resolve uncertainty and support clinical decision-making. Alternatively, after the genomic instability prediction platform 115 generates a predicted genomic instability status, the other assays 108 can be used to perform confirmatory testing to confirm the predicted results. Confirmatory testing may include PCR-based fragment analysis for microsatellite loci to validate suspected MSI, methylation-specific assays to assess the epigenetic status of promoter regions such as MLH1, and Sanger sequencing to confirm specific variants identified by broader panels. Additional tests, such as loss of heterozygosity analysis, chromosomal microarray, or functional assays measuring DNA repair capacity, may also be performed to further clarify or confirm the genomic instability status. The inclusion of other assays 108 is also valuable for challenging samples, such as those with degraded DNA, low tumor content, or limited tissue availability, where primary assays may be compromised.

The genomic instability testing platform 102 interacts with other components of the environment 100 to ensure streamlined data flow and comprehensive analysis. For example, test orders and results can be exchanged with the client device 105, which may be used by clinicians or laboratory personnel to initiate testing, review reports, manage patient records, or develop personalized treatment plans. Processed data and intermediate results from the genomic instability testing platform 102 can also be transmitted to the genomic instability prediction platform 115 to predict genomic instability status when traditional assays are inconclusive. In addition, the platform may communicate with the server 135 (e.g., the data repository 110) for data storage, processing, and secure management of patient information or the CGIP platform 145 to provide the multi-omics data. All communication between the genomic instability testing platform 102 and other components may occur via the network 120.

A client device 105 is an electronic device including hardware, software, or embedded logic components or a combination of two or more such components and capable of interacting with the server 135 or a unit thereof (e.g., the data repository 110, the genomic instability prediction platform 115) and the CGIP platform 145 or a unit thereof (e.g., the genomic profiling module 160, the immune profiling module 165), optionally via the network 120. The client device 105 may include various types of computing systems such as portable handheld devices such as cell phones, general purpose computers such as personal computers and laptops, workstation computers, wearable devices, gaming systems, thin clients, various messaging devices, sensors or other sensing devices, and the like. These computing devices may run various types and versions of software applications and operating systems (e.g., Microsoft Windows®, Apple Macintosh®, UNIX® or UNIX-like operating systems, Linux or Linux-like operating systems such as Google Chrome™ OS) including various mobile operating systems (e.g., Microsoft Windows Mobile®, iOS®, Windows Phone®, Android™, BlackBerry®, Palm OS®). Portable handheld devices may include cellular phones, smartphones, (e.g., an iPhone), tablets (e.g., iPad®), personal digital assistants (PDAs), and the like. Wearable devices may include Google Glass® head mounted display, and other devices. The client device 105 may be capable of executing various different applications such as various Internet-related apps, communication applications (e.g., E-mail applications, short message service (SMS) applications) and may use various communication protocols. This disclosure contemplates any suitable client device 105 configured to generate and output product target discovery content to a user. For example, users may use client device 105 to execute one or more applications, which may generate one or more discovery or storage requests that may then be serviced in accordance with the teachings of this disclosure. The client device 105 may provide an interface 130 (e.g., a graphical user interface) that enables a user of the client device 105 to interact with the client device 105. The client device 105 may also output information to the user via this interface 130 (e.g., displaying a report). Although FIG. 1 depicts only one client device 105, any number of client devices 105 may be supported.

The client device 105 is capable of inputting data, generating data, and receiving data. The client device 105 may be operated by clinicians, laboratory personnel, or researchers who require access to testing services, results, or advanced analytics. For example, a user of the client device 105 may initiate a request to perform an initial genomic instability screening by interacting with the interface 130, which is designed for intuitive order entry and sample tracking. This request can direct the genomic instability testing platform 102 to process a patient sample using the NGS assays 104, the IHC lab 106, or other assays 108.

The client device 105 can also be used to request AI-based genomic instability prediction through the genomic instability prediction platform 115. In this scenario, the user may select a specific sample or cohort and trigger advanced computational analyses, for example when the initial testing has failed or provided inconclusive results. The client device 105 can transmit these requests and receive prediction results or detailed reports seamlessly, allowing users to act on the information quickly in a clinical or research setting. In addition, the client device 105 may communicate with the CGIP platform 145 through the network 120 to request or retrieve extensive genomic profiling data and gene expression data for a particular sample or a set of training samples, which supports both real-time clinical decision-making and retrospective data analysis, such as refining machine learning models or validating predictive features. The secure connection provided by the network 120 ensures that all communications between the client device 105 and other components are reliable, fast, and compliant with data privacy requirements.

A data repository 110 is a data storage entity (or sometimes entities) into which data has been specifically partitioned for an analytical or reporting purpose. The data repository 110 may be used to store data and other information generated or used by the genomic instability testing platform 102, the genomic instability prediction platform 115, the client device 105, and/or the CGIP platform 145. For example, one or more of the data repositories 110 may be used to store data and information to be used as input into the genomic instability prediction platform 115 for generating a genomic instability report. The data repositories 110 may reside in a variety of locations including the server 135. For example, a data repository used by a server 135 may be local to the server 135 or may be remote from the server 135 and in communication with the server 135 via a network-based or dedicated connection of network 120. Data repositories 110 may be of different types or of the same type. In certain examples, a data repository 110 may be a database which is an organized collection of data stored and accessed electronically from one or more storage devices such as one or more servers 135. The one or more servers 135 may be configured to execute a database application that provides database services to other computer programs or to computing devices (e.g., client device 105 and genomic instability prediction platform 115) within the computing environment 100, as defined by a client-server model. One or more of these databases may be adapted to enable storage, update, and retrieval of data to and from the database in response to SQL-formatted commands or like programming language that is used to manage databases and perform various operations on the data within them.

The genomic instability prediction platform 115 comprises a set of tools 140 for the purpose of analyzing and visualizing data (e.g., data stored in the data repository 110, data generated by the genomic instability testing platform 102 or the CGIP platform 145, or the data sent from the client device 105) and a genomic instability caller 170. The genomic instability prediction platform 115 is used to execute a process to provide genomic instability predictions based on CGIP data. In the exemplary configuration depicted in FIG. 1, the set of tools 140 include two units: a preprocessing unit 150 and a feature extractor 155. The preprocessing unit 150 is capable of loading, processing, and saving data (e.g., accessed from the data repository 110) to be used by the preprocessing unit 150 itself and the feature extractor 155. The feature extractor 155 uses the processed data to identify genomic instability related features based on the CGIP data. The genomic instability caller 170 trains machine learning models and uses the trained machine learning models to make genomic instability callings.

In some embodiments, the genomic instability prediction platform 115 is used together with the genomic instability testing platform 102 and/or the CGIP platform 145 to: (i) generate genomic profiling data for a patient sample (for whole genome or for regions of interest), (ii) generate immune profiling data for the sample; (iii) obtain demographic information, configuration files, and reference data (e.g., from the data repository 110), (iv) determine small-scale genomic features and feature values associated with the genomic instability status based on the genomic profiling data and/or data from (iii), (v) determine gene expression changes associated with the genomic instability status based on the immune profiling data and/or data from (iii), (vi) select genomic instability predictor features from features in (iv) and (v) using a feature selection algorithm, (vii) train a machine learning model to predict genomic instability status based on genomic instability predictor features, (viii) predict a genomic instability status using the trained machine learning model, and (ix) output information and/or reports. The genomic instability prediction platform 115 may also be configured to train multiple machine learning models using different machine learning algorithms and select a trained model with desired accuracy (e.g., sensitivity, specificity, positive predictive value (PPV), negative predictive value (NPV), balanced accuracy, F1 score, and/or Receiver Operating Characteristic-Area Under the Curve (ROC-AUC) value).

The genomic instability prediction platform 115 may reside in a variety of locations including the server 135. For example, a genomic instability prediction platform 115 used by a server 135 may be local to the server 135 or may be remote from the server 135 and in communication with the server 135 via a network-based or dedicated connection of network 120. The genomic instability prediction platform 115 may be of different configurations or of the same configuration.

The server 135 may be adapted to run one or more services or software applications that enable one or more embodiments described in this disclosure. In certain instances, server 135 may also provide other services or software applications that may include non-virtual and virtual environments. In some examples, these services may be offered as web-based or cloud services, such as under a Software as a Service (SaaS) model to the users of client device 105. Users operating client device 105 may in turn utilize one or more client applications to interact with server 135 to utilize the services provided by these components (e.g., database and rescue applications). In the configuration depicted in FIG. 1, server 135 may include one or more components that implement the functions performed by server 135. These components may include software components that may be executed by one or more processors, hardware components, or combinations thereof. It should be appreciated that various different device configurations are possible, which may be different from computing environment 100. The example shown in FIG. 1 is thus one example of a computing environment and is not intended to be limiting.

Server 135 may be composed of one or more general purpose computers, specialized server computers (including, by way of example, PC (personal computer) servers, UNIX® servers, mid-range servers, mainframe computers, rack-mounted servers, etc.), server farms, server clusters, or any other appropriate arrangement and/or combination. Server 135 may include one or more virtual machines running virtual operating systems, or other computing architectures involving virtualization such as one or more flexible pools of logical storage devices that may be virtualized to maintain virtual storage devices for the server. In various instances, server 135 may be adapted to run one or more services or software applications that provide the functionality described in the foregoing disclosure. In some embodiments, the server 135 is a physical or virtual machine for hosting applications, storing data, managing databases, or facilitating communication between systems that provides computing resources and services to other devices (clients) on a network. In some embodiments, the server 135 is an on-premises server or a cloud-based server.

The computing systems in server 135 may run one or more operating systems including any of those discussed above, as well as any commercially available server operating system. Server 135 may also run any of a variety of additional server applications and/or mid-tier applications, including HTTP (hypertext transport protocol) servers, FTP (file transfer protocol) servers, CGI (common gateway interface) servers, JAVA® servers, database servers, and the like. Exemplary database servers include without limitation those commercially available from Oracle®, Microsoft®, Sybase®, IBM® (International Business Machines), and the like.

In some implementations, server 135 may include one or more applications to analyze and consolidate data feeds and/or data updates received from users of client devices 105. As an example, data feeds and/or data updates may include, but are not limited to, in vivo feeds, in silico feeds, or real-time updates received from public studies, user studies, one or more third party information sources, and data streams (continuous, batch, or periodic), which may include real-time events related to sensor data applications, biological system monitoring, and the like. Server 135 may also include one or more applications to display the data feeds, data updates, and/or real-time events via one or more display devices of client devices 105.

The CGIP platform 145 is configured to obtain, generate, and process multi-omics data by accessing public databases to retrieve the data or performing a wide range of sequencing tasks and analytical assays. These assays may include the assays conducted within the genomic instability testing platform 102, including next-generation sequencing (NGS), Sanger sequencing, droplet digital PCR (ddPCR), quantitative PCR (qPCR), RNA sequencing (RNA-Seq), immunohistochemistry (IHC), and other functional or confirmatory assays, such as homologous recombination (HR) repair assays, drug sensitivity assays, or synergy assays. The CGIP platform 145 incorporates integrated data processing pipelines that manage raw data acquisition, quality control, normalization, data imputation, and downstream analysis, ensuring that the resulting multi-omics datasets are both complete and accurate for further interpretation and decision-making. This platform enables comprehensive genomic and immune profiling by integrating multiple sequencing and analysis technologies. The CGIP platform 145 may operate fully automatically once biological samples are loaded, or semi-automatically with the assistance of a practitioner, depending on the workflow and complexity of the analysis. In some embodiments, the CGIP platform 145 is capable of performing same or overlapping functions with the genomic instability testing platform 102.

As depicted in FIG. 1, the CGIP platform 145 includes a genomic profiling module 160 and an immune profiling module 165. The genomic profiling module 160 is designed to perform DNA sequencing tasks, generating high-resolution genomic profiling data, including information on mutations, copy number variations, structural rearrangements, and other genomic instability events. The immune profiling module 165 focuses on RNA sequencing to provide immune profiling data, such as transcriptomic signatures and the expression levels of immune-related genes, enabling insights into tumor-immune interactions and immune system modulation. In some embodiments, the CGIP platform 145 can include additional specialized modules or units to expand its capabilities. For example, a third-generation sequencing (TGS) unit may be integrated to the genomic profiling module 160 to perform further sequencing techniques, such as single molecule real-time (SMRT) sequencing or nanopore sequencing, which allow for the analysis of long DNA or RNA reads with high accuracy. The CGIP platform 145 may also include a pyrosequencing module, an Ion Torrent sequencing module, a PCR module, a sequencing by ligation (SOLID) module, or an IHC imaging module. In certain configurations, the CGIP platform 145 can be configured to consolidate sequencing and profiling capabilities into a single module and provide consolidated profiling information. Integrated data processing software within the CGIP platform 145 manages the aggregation of data from these modules, standardizes data formats, and applies normalization, data imputation, and quality control to generate coherent outputs suitable for downstream use.

Data processing steps such as filtering, normalization, imputation, and feature engineering may be performed within the CGIP platform 145, within the genomic instability prediction platform 115, or at both locations as needed. Filtering may involve removing low-quality sequencing reads, discarding base calls with low Phred quality scores, or eliminating PCR duplicates that can introduce bias into the dataset. For example, reads with ambiguous nucleotides or those failing to map confidently to a reference genome may be excluded to enhance the accuracy of variant detection. Normalization is performed to correct for technical variability, such as differences in sequencing depth, library preparation protocols, or batch effects between different runs. For instance, RNA-Seq data may be normalized using methods like TPM (Transcripts Per Million) or FPKM (Fragments Per Kilobase of transcript per Million mapped reads), while DNA variant calls may be adjusted for coverage depth to ensure fair comparison across samples. Imputation addresses missing values by estimating or filling in incomplete data points. As an example, if a particular gene's expression value is absent from an RNA-Seq dataset due to low transcript abundance or technical dropout, statistical methods such as multivariate imputation by chained equations (MICE) or k-nearest neighbors can be used to estimate the missing value based on observed patterns in the rest of the data. Feature engineering transforms raw data into informative variables that can enhance the predictive power of machine learning models. For example, tumor mutational burden (TMB) may be calculated from the variant call files, immune gene expression may be summarized into composite inflammation scores, or pathway activity scores may be derived from sets of related genes. Additional examples include encoding the presence or absence of specific driver mutations, aggregating copy number changes into segment-level alterations, or constructing binary indicators for loss of heterozygosity events.

In some embodiments, the candidate mutation information or the gene expression information may be sent back to the CGIP platform 145 to perform confirmatory sequencing or testing, for example, using the genomic profiling module 160 or immune profiling module 165. For example, for targeted mutation confirmation, a Sanger sequencing may be performed by the genomic profiling module 160, and to confirm gene expression levels, qPCR can be performed by the immune profiling module 165. The candidate mutation information and the gene expression information may also be communicated to the user of the client device 105 and the user may decide whether to perform confirmation testing or determine personalized treatments. In some embodiments, the genomic profiling/gene expression data and the confirmation data are used together to determine if the sample comprises the certain mutations and its gene expression levels, or if a subject where the sample obtained is predicted to have a genomic instability condition (e.g., HRD positive or MSI-high). The genomic instability condition information may be transmitted to the client device 105 via the network 120. The data (e.g., the genomic profiling/gene expression data, the confirmation data, the feature information, the demographic information, and/or genomic instability condition) may also be sent and stored in the data repository 110. Automated data processing modules within the CGIP platform 145 and the server 135 handle the integration, validation, and storage of all incoming and outgoing data, providing traceability and audit trails for clinical and research use.

In some embodiments, the genomic profiling module 160 performs a nucleic acid extraction process to isolate high-quality nucleic acid from a biological sample. This process may involve using automated extraction systems or manual protocols, depending on the sample type and throughput needs. Extraction reagents and spin columns, magnetic beads, or organic solvents may be used to purify the DNA or RNA from whole blood, tissue, FFPE samples, or other biological matrices. For example, thin sections of FFPE tissue may be obtained and placed into appropriate microtubes. Then, deparaffinization is performed by incubating the tissue sections with xylene or another organic solvent to dissolve and remove the paraffin. After removal of the paraffin, the samples are washed with ethanol to eliminate residual solvent. In some instances, the tissue is subjected to rehydration using graded ethanol solutions or buffer washes. The processed samples may undergo a proteinase K digestion step, which breaks down crosslinked proteins and helps release the nucleic acids from the fixed tissue matrix. Incubation at elevated temperatures may be applied to further reverse crosslinking between nucleic acids and proteins. At last, nucleic acid purification is achieved using spin columns, magnetic beads, or organic extraction methods to separate the DNA or RNA from other cellular components.

Quality and integrity of the extracted nucleic acid can be assessed by spectrophotometry, fluorometric assays, or gel electrophoresis before proceeding. For example, spectrophotometric analysis using instruments such as a NanoDrop can provide measurements of nucleic acid concentration and assess purity ratios to identify contamination by proteins or solvents. Fluorometric assays and gel electrophoresis, including agarose gel or capillary systems, can be used to visualize the size distribution of the nucleic acid fragments and to evaluate the extent of degradation or shearing. Bioanalyzer or TapeStation instruments may also be employed to determine RNA integrity number (RIN) values or DNA integrity scores, providing further confidence in sample suitability for downstream applications.

This may be followed by fragmentation, where the extracted nucleic acids are broken into smaller, more manageable pieces. Mechanical shearing methods, such as sonication (using instruments like Covaris) or nebulization, physically break the nucleic acid strands into fragments of a defined size range. Enzymatic digestion utilizes specific nucleases to cleave the nucleic acids at random or targeted locations, while maintaining controlled fragment lengths. Some protocols may combine both approaches or use tagmentation, in which a transposase simultaneously fragments and tags the DNA with sequencing adapters in a single reaction. The chosen fragmentation method is often selected based on the desired insert size, input quality, and compatibility with downstream library preparation workflows.

In some embodiments, the nucleic acid is cell-free nucleic acid and fragmentation may not be required. Cell-free DNA or RNA, such as that isolated from plasma or other body fluids, is typically already present in short fragments due to natural degradation processes. In these cases, the extracted nucleic acid can proceed directly to library preparation without additional fragmentation, thereby preserving as much material as possible for sensitive downstream analysis.

The fragmented nucleic acid or cell-free nucleic acid is then prepared for sequencing as part of a library preparation process. This process may include the ligation of sequencing adapters, which adapters are short, double-stranded DNA sequences (or single-stranded nucleic acid sequences) that are ligated to the ends of the fragments, allowing them to bind to the sequencing flow cell and facilitate amplification. The library preparation process may also involve additional steps to ensure that the fragments are of the appropriate size and concentration for sequencing. This can include size selection, where fragments of a specific length are isolated using gel electrophoresis or magnetic beads. The library can be cleaned to remove adapter dimers and unwanted fragments. The prepared library is then quantified and quality-checked using techniques such as quantitative PCR (qPCR) or bioanalyzer assays to ensure that it meets the requirements for sequencing. In some instances, the wet-lab procedures are performed by a trained practitioner.

Once the library is ready, it can be loaded onto the CGIP platform 145 or the genomic profiling module 160. Different sequencing platforms may have their own sequencing chemistries and technologies, generally involving the attachment of the library fragments to a solid surface, amplification to create clusters or colonies of identical sequences, and sequencing-by-synthesis or other methods to read the nucleotide sequence of each fragment. The sequencing process performed by the genomic profiling module 160 generates massive amounts of data (e.g., millions to billions of sequence reads or raw data), which can be then transferred to the server 135 or the genomic instability prediction platform 115 for analysis. In some instances, the genomic profiling module 160 or another component of the CGIP platform 145 may analyze, process, or manage the sequencing data. For example, bioinformatics tools and algorithms may be employed to process raw sequencing data, which includes base calling, quality control, read alignment, and variant calling. High-performance computing systems and cloud-based platforms are often used to handle the computationally intensive tasks of sequence alignment and data analysis. Additionally, specialized software pipelines are used to assemble the sequenced reads into complete genomes or to identify genetic variants. The integration of artificial intelligence and machine learning algorithms may be further adopted to enhance the accuracy and efficiency of data analysis, enabling the identification of genetic markers and potential therapeutic targets.

The immune profiling module 165 is configured to generate comprehensive gene expression data by analyzing RNA extracted from biological samples. The process begins with sample preparation, where biological materials are collected through biopsy, surgical resection, or blood draws. RNA is then extracted using standardized protocols that involve cell lysis, removal of contaminants like proteins and DNA, and purification of RNA. RNA extraction may use silica column kits, magnetic beads, or phenol-chloroform extraction, depending on the desired yield and purity. The quality and integrity of the extracted RNA can be assessed. Additionally, RNA concentration may be measured using spectrophotometers or fluorometric assays.

Once the RNA is prepared, it may undergo reverse transcription to convert it into complementary DNA (cDNA). Reverse transcriptase enzymes and random hexamer or oligo-dT primers may be used for this conversion. The cDNA is fragmented, and sequencing adapters are ligated to facilitate amplification and compatibility with sequencing platforms. Unique molecular barcodes or indices can also be added during this step to allow for sample multiplexing, enabling simultaneous sequencing of multiple samples. The prepared cDNA libraries are then amplified using polymerase chain reaction (PCR) to generate sufficient quantities for sequencing. Target enrichment protocols may also be applied to focus on immune-related transcripts to enhance the specificity of immune profiling. For example, hybrid capture or amplicon-based enrichment methods may be employed to selectively amplify genes of interest, such as those involved in immune response or checkpoint pathways.

The sequencing process can be carried out on sequencing units integrated into the immune profiling module 165. These units read the cDNA fragments and generate raw sequencing data in digital data formats such as FASTQ. The raw data undergoes quality control to remove low-quality reads, adapter sequences, and other artifacts. Quality metrics such as Phred score distribution, duplication rate, and adapter content can be checked. High-quality reads can then be aligned to a reference genome or transcriptome using bioinformatics tools to map the reads to specific genes or transcripts. Once alignment is complete, the immune profiling module 165 may quantify gene expression levels by counting the number of reads mapped to each gene, and the resulting raw counts can be further normalized to account for factors such as sequencing depth and transcript length. Common normalization methods include fragments per kilobase of transcript per million mapped reads (FPKM) or transcripts per million (TPM). Alternative normalization approaches, such as DESeq2's median-of-ratios or edgeR's Trimmed Mean of M-values (edgeR's TMM), may be also used. The raw data or normalized data is then subjected to further analysis (e.g., sending to the genomic instability prediction platform 115 for making genomic instability calls).

In some embodiments, further analysis may also be performed by the immune profiling module 165 to understand the functional implications of these gene expression level changes. Pathway analysis tools can be used to identify biological pathways and immune-related processes, such as immune activation, inflammation, or immune evasion. Immune signature profiling can be also conducted to assess specific immune features, such as T-cell activation, interferon response, or checkpoint inhibition pathways, providing insights into the immune landscape of the sample. Algorithms may calculate cytolytic scores, estimate immune cell populations, or assess immune checkpoint gene expression. In some embodiments, the immune profiling data can be processed using AI-based algorithms to predict immune responses, identify potential biomarkers, or stratify patients for personalized therapies.

In some embodiments, Sanger sequencing can be performed by the CGIP platform 145 to confirm the nucleotide sequences of nucleic acid molecules. Sanger sequencing utilizes a method known as chain termination, which synthesizes a complementary DNA strand using a single-stranded DNA template, a DNA polymerase enzyme, and a mixture of normal deoxynucleotides (dNTPs) and chain-terminating dideoxynucleotides (ddNTPs). The ddNTPs are modified nucleotides that lack a 3′ hydroxyl group, preventing further elongation of the DNA strand once incorporated. These ddNTPs are fluorescently or radioactively labeled, enabling the detection of terminated fragments. By incorporating a small proportion of ddNTPs in the reaction, a mixture of DNA fragments of varying lengths is generated, each ending at a specific nucleotide. The DNA fragments are then separated by size using capillary electrophoresis, where an electric field is applied to a capillary tube filled with a polymer matrix. Smaller fragments migrate faster through the capillary, while larger fragments move more slowly. As the fragments pass through a detector, the fluorescent or radioactive labels are identified, and the sequence of the DNA is determined by analyzing the order of the labeled fragments. Sanger sequencing may be used for confirmation of specific variants detected in NGS data, for quality control of cloned constructs, or for targeted hotspot analysis. The sequence data generated by Sanger sequencing can also be sent to the server 135 or the genomic instability prediction platform 115 for further analysis. The CGIP platform 145 can compile and interpret Sanger sequencing data to reconstruct the original DNA sequence, validate sequences obtained from the genomic profiling module 160, or detect variants in biological materials.

In some embodiments, gene expression data can be confirmed by the CGIP platform 145 through a series of complementary molecular and analytical techniques. For example, a quantitative PCR (qPCR) module can be used to measure the expression level of specific genes by amplifying cDNA derived from RNA. The qPCR process begins with the extraction of total RNA from biological samples, followed by reverse transcription to convert the RNA into cDNA using a reverse transcriptase enzyme. The cDNA is then used as a template in a qPCR reaction, which includes gene-specific primers, a DNA polymerase enzyme, and a fluorescent dye or probe to monitor DNA amplification in real time. The fluorescence intensity increases proportionally with the amount of amplified DNA, allowing precise quantification of gene expression. The qPCR results are analyzed by comparing the amplification cycle threshold (Ct) values of the target genes to those of reference (housekeeping) genes, which serve as internal controls for normalization. The normalized expression data can then be compared to baseline or control samples to confirm whether specific genes are upregulated or downregulated under certain conditions.

In addition to qPCR, the CGIP platform 145 may also confirm gene expression data using complementary techniques such as Northern blotting, which involves the separation of RNA on a gel followed by hybridization with labeled probes to detect specific transcripts. Protein-level validation techniques, such as Western blotting or IHC, can further confirm gene expression by correlating RNA levels with the abundance of the corresponding protein. For higher-throughput validation, RNA sequencing (RNA-Seq) data can be cross-referenced with publicly available datasets, such as The Cancer Genome Atlas (TCGA) or Gene Expression Omnibus (GEO), to ensure consistency and accuracy.

To further illustrate the operation of the described environment, consider a comprehensive workflow for determining an MSI status in a cancer patient. After a clinician collects a tumor biopsy sample from the patient, the sample may be processed by the genomic instability testing platform 102 or the CGIP platform 145, both of which are capable of handling biological samples and generating multi-omics data relevant to genomic instability determination. The sample is submitted for genomic instability testing through the client device 105, which serves as the entry point for initiating test requests and tracking samples within the environment. DNA and RNA extraction is conducted using automated systems within the NGS assays 104 of the genomic instability testing platform 102 or the genomic profiling module 160 of the CGIP platform 145. DNA sequencing is performed to detect genomic alterations, including single nucleotide variants, indels, copy number variations, and gene fusions, while RNA sequencing profiles the expression of immune-related genes implicated in MSI status. In parallel, a portion of the sample may be processed in the IHC lab 106 of the genomic instability testing platform 102, where tissue sections are stained with antibodies targeting mismatch repair proteins such as MLH1, MSH2, MSH6, and PMS2. Visualization of these stained slides using digital scanners provides evidence for the presence or absence of genomic instability. All resulting raw and processed data, including sequencing reads, gene expression values, and IHC findings, are securely transmitted to the data repository 110, ensuring traceability, secure storage, and accessibility for downstream analysis by the genomic instability prediction platform 115 and the CGIP platform 145.

If the initial NGS and IHC results are inconclusive or conflicting regarding the patient's MSI status, the clinician may use the client device 105 to initiate an AI-based genomic instability prediction workflow. The genomic instability prediction platform 115 retrieves multi-omics data from the data repository 110, including both genomic alteration data from DNA sequencing and immune gene expression profiles from RNA sequencing. The platform preprocesses the data, applies normalization to address technical variability, and imputes missing values using methods such as MICE or regression-based approaches. Relevant features (relevant predictors of MSI status) are extracted from the multi-omics data. A machine learning model trained on annotated cancer samples with established MSI status uses these extracted features to generate a prediction for the patient. The predicted MSI status, alongside supporting data, feature importance, and model performance metrics, is compiled into a comprehensive testing report and securely transmitted to the clinician's interface via the client device 105 for clinical decision-making. If the AI-based analysis suggests a high likelihood of MSI-high status, the report may recommend confirmatory tests, such as MLH1 promoter methylation analysis, Sanger sequencing, or other assays available through the genomic instability testing platform 102 or the CGIP platform 145, to validate the prediction. Throughout this workflow, all data flows and actions are securely managed and auditable within the integrated environment, supporting compliance with privacy and security standards and enabling personalized patient care.

### III. GENOMIC INSTABILITY PREDICTION WORKFLOW

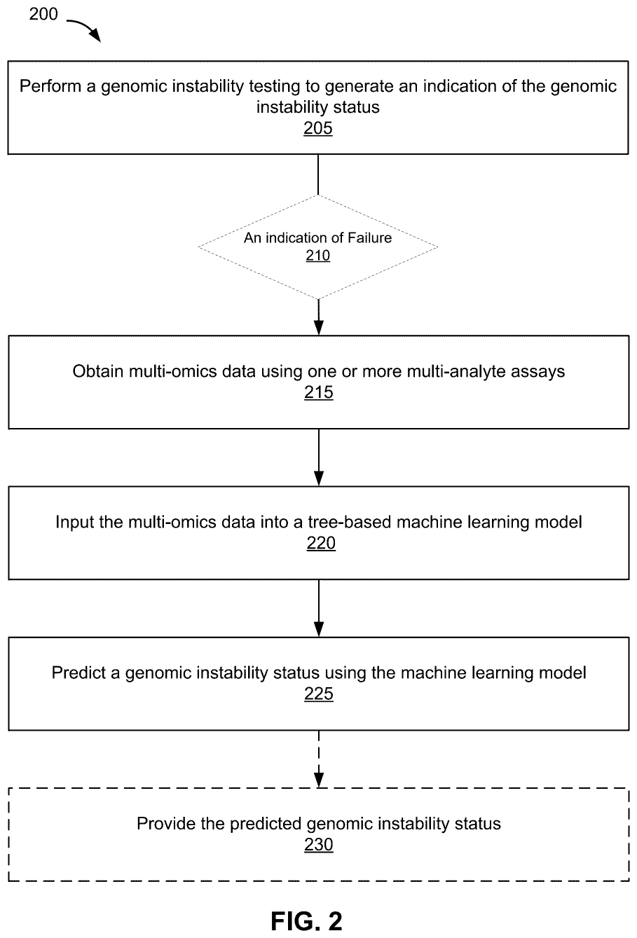

FIG. 2 shows an exemplary workflow 200 for performing a genomic instability prediction assay that enables the prediction of genomic instability status for a biological sample in accordance with various embodiments. This workflow 200 is designed to support both conventional and AI-based approaches for detecting genomic instability, such as MSI and HRD, particularly in cases where traditional genomic instability testing may not yield a definitive result. The workflow 200 depicted in FIG. 2 may be implemented in software (e.g., code, instructions, program) executed by one or more processing units (e.g., processors, cores) of the respective systems, hardware, or combinations thereof (e.g., the intelligent selection machine). The software may be stored on a non-transitory store medium (e.g., on a memory device). The workflow 200 can be performed using the computing environment 100 described with respect to FIG. 1. The workflow 200 presented in FIG. 2 and described below is intended to be illustrative and non-limiting. Although FIG. 2 depicts the various processing steps occurring in a particular sequence or order, this is not intended to be limiting. In certain alternative embodiments, the steps may be performed in some different orders, or some steps may also be performed in parallel.

At block 205, the workflow 200 begins by performing a genomic instability testing on a biological sample to generate an initial indication of the sample's genomic instability status. In some embodiments, the genomic instability is MSI or HRD, and the initial indication includes MSI-high, microsatellite stable (MSS), HRD-high, and/or HRD-low. This initial test can be conducted using the genomic instability testing platform 102 as described in FIG. 1. The genomic instability testing platform 102 may use the NGS assays 104, the IHC lab 106, and other assays 108 to assess markers such as MSI or HRD to determine the initial indication. Standard methods performed by these modules may include NGS for detecting genomic alterations, PCR-based assays for microsatellite analysis, or IHC for evaluating protein markers relevant to genomic instability. If the genomic instability testing yields a conclusive result, the workflow 200 may be completed by generating a testing report, which can be managed or communicated through components such as the server 135 or the client device 105 within the environment 100. If the genomic instability testing yields an inconclusive result or a test failure (see decision block 210), the workflow 200 advances to an AI-based genomic instability prediction, leveraging multi-omics data and advanced machine learning models implemented on platforms such as the genomic instability prediction platform 115 and the CGIP platform 145. If the genomic instability testing yields a conclusive result (e.g., MSI-high or MSS), the workflow 200 can also advance to the AI-based genomic instability prediction to confirm the original genomic instability testing result.

The decision block 210 evaluates the result generated from the genomic instability testing performed at block 205 to determine whether the outcome is conclusive. The result can be assessed based on predefined criteria for reportability and clarity. If the outcome is definitive, such as when a clear MSI-high, MSS, HRD-high, or HRD-low status is assigned, the workflow may conclude and the result can be recorded or communicated to downstream systems or clinical users. Alternatively, the workflow 200 may advance to AI-based genomic instability prediction to confirm the original genomic instability testing result. However, if the conventional testing yields an indication of failure, the workflow advances to the next phase. In some embodiments, the decision block 210 is optional and the workflow 200 can go directly to block 215.

For example, MSI status can be established using either PCR-based fragment analysis, NGS-based approaches, or IHC for MMR proteins. In PCR-based fragment analysis, a panel of microsatellite markers (e.g., 5 markers include BAT-25, BAT-26, D2S123, D5S346, and D17S250) is amplified from tumor and, if available, matched normal DNA. The lengths of the microsatellite repeats are compared between tumor and normal. If two or more of the five markers show instability, the sample is classified as MSI-high. If none show instability, it is classified as MSS. For example, if three out of five Bethesda markers display size shifts, the tumor is considered MSI-high; if all five are stable, the tumor is MSS. If only one marker shows instability, the indication is inconclusive or failure.

For NGS-based MSI detection, sequencing panels may evaluate hundreds of microsatellite loci across the genome. Analytical algorithms, such as MSIsensor, are used to calculate the proportion of loci that are unstable in the tumor sample compared to a matched normal or reference dataset. If the computed score exceeds a validated threshold, for example, an MSIsensor score greater than 10, the case is classified as MSI-high. If the score falls below a lower cutoff, such as less than 3, the sample is classified as MSS. When the score is between these thresholds, or when the result cannot be confidently assigned due to data quality or coverage issues, the outcome is designated as inconclusive or as a test failure.

HRD status can be determined by calculating the genomic instability score that reflects the sum of large-scale chromosomal events, such as loss of heterozygosity (LOH), telomeric allelic imbalance (TAI), and large-scale state transitions (LST). Commercial assays may use a validated cutoff value (e.g., set at 42) to distinguish between HRD-high and HRD-low classifications. If a sample's HRD score is 42 or greater, it is considered HRD-high; if the score is below 42, it is considered HRD-low. Additionally, the identification of pathogenic mutations in genes such as BRCA1 or BRCA2 may also support a classification of HRD-high, particularly when the overall genomic instability score is elevated. For example, a sample with an HRD score of 48, or one that harbors a pathogenic BRCA1 mutation along with high LOH, would be classified as HRD-high. Conversely, a sample with an HRD score of 27, or one lacking BRCA1/2 mutations and demonstrating low genomic instability, would be classified as HRD-low. These criteria provide a standardized framework for interpreting genomic instability in clinical and research settings, although specific thresholds may vary depending on the assay or clinical guidelines. If a sample's HRD score is close to the cutoff and other supporting evidence is ambiguous, the result may be designated as inconclusive or failure. These examples and thresholds are provided solely for the purpose of illustration and are not intended to be limiting. Actual cutoff values and interpretation criteria may vary by laboratory protocol, assay design, or clinical guideline.

An indication of failure can occur for several reasons. For instance, the test may be inconclusive due to technical limitations, such as low DNA quantity or quality, degraded RNA, insufficient tumor content, or assay artifacts that prevent a reliable result. Ambiguous findings may also result from borderline values, uninterpretable sequencing data, or conflicting results between different assay modalities. Additionally, sample-specific factors like contamination or the presence of inhibitors may interfere with assay performance. In these situations, the workflow 200 classifies the result as a failure or indeterminate status. Upon recognizing this indication of failure, the workflow 200 initiates an alternative approach.

At block 215, multi-omics data for the subject is obtained using one or more multi-analyte assays. The multi-omics data may include a combination of genomic profiling data (genomic alterations such as SNVs, indels, CNVs, gene fusions, splice variants, and TMB) and immune profiling data (e.g., gene expression profiles for immune-related genes or inflammation signatures). Part or all of the multi-omic data can be generated using platforms such as the genomic instability testing platform 102 or the CGIP platform 145 as described with respect to FIG. 1. The biological samples used for multi-omics profiling may include blood, tissue, or other materials, depending on the clinical context. In some embodiments, the one or more multi-analyte assays comprise DNA sequencing and RNA sequencing, and wherein the multi-omics data comprises genomic alteration data for a first set of genes obtained using the DNA sequencing and expression data for a second set of immune genes obtained using the RNA sequencing. In some embodiments, the one or more multi-analyte assays include the genomic instability testing performed at block 205 and the multi-omics data includes data generated at block 205. In some embodiments, the one or more multi-analyte assays may be a single CGIP assay.

In some embodiments, the multi-omics data for the subject may further comprise immunohistochemistry data, cell proliferation data, tumor inflammation data, and/or cancer testis antigen burden data. The immunohistochemistry data may include results from antibody-based staining assays performed on tissue samples. For example, the immunohistochemistry data can comprise PD-L1 immunohistochemistry data, which reflects the expression of PD-L1 protein in tumor or immune cells and is relevant for assessing immune checkpoint status. The cell proliferation data may include the Ki-67 proliferation index, which quantifies the percentage of cells expressing the Ki-67 protein and serves as a marker of tumor growth rate. The tumor inflammation data may consist of gene expression signatures associated with tumor inflammation, such as expression levels of cytokines, chemokines, or immune activation genes. The cancer testis antigen burden data may include the expression levels of one or more cancer testis antigens, which are proteins typically restricted to germ cells but aberrantly expressed in certain tumors.

To obtain these data types, several laboratory methods may be employed. In some embodiments, a formalin-fixed, paraffin-embedded (FFPE) tissue sample is stained with an antibody specific to a protein of interest. The stained FFPE tissue sample is then evaluated by light microscopy or digital image analysis to obtain the immunohistochemistry data, the cell proliferation data, the tumor inflammation data, and the cancer testis antigen burden data. For example, PD-L1 expression, Ki-67 index, immune marker staining, and cancer testis antigen detection can all be assessed using this approach.

Alternatively or additionally, RNA may be extracted from the FFPE tissue sample and subjected to gene expression profiling using RNA sequencing or quantitative PCR. These molecular assays can be used to obtain the cell proliferation data by measuring the expression of proliferation markers such as Ki-67, the tumor inflammation data by quantifying inflammation-related gene signatures, and the cancer testis antigen burden data by assessing the expression of cancer testis antigen genes. This molecular profiling enables a quantitative and high-throughput assessment of additional biological features associated with genomic instability.

The one or more multi-analyte assays may also be performed at block 215 to generate the CGIP data. For example, a biopsy or a tissue sample can be obtained from the subject, and DNA, RNA, proteins, or metabolites may be extracted from the same sample. In some embodiments, the sample is the same sample as the biological sample where the genomic instability testing is performed on (e.g., at block 205) or a portion thereof. An NGS assay may be performed to detect DNA alterations, a transcriptomic profiling assay may measure RNA expression levels using RNA sequencing (RNA-Seq), and proteomic analysis, mass spectrometry, or antibody-based assays may be performed to quantify protein abundance and post-translational modifications. Metabolomic profiling assays may also be performed using techniques such as liquid chromatography-mass spectrometry (LC-MS) to identify and quantify metabolites that reflect cellular metabolic states.

In some embodiments, a blood sample may be obtained from the subject and cell-free DNA (cfDNA) may be extracted from the blood sample for genomic and immune profiling. Blood may be collected in specialized tubes to stabilize cfDNA, then centrifuged to isolate plasma. The cfDNA is extracted from the plasma and used for both analyses. Genomic profiling can be performed using NGS to identify mutations, copy number changes, and other tumor-specific alterations. Immune profiling can analyze the same cfDNA for epigenetic markers, such as DNA methylation, or immune signatures, to infer the activity of immune-related genes and pathways.

In some embodiments, multiple samples may also be obtained from the subject to perform the necessary multi-analyte assays. These samples can be of the same type, such as two tissue or blood samples, or of different types, such as a combination of solid tissues (tumor biopsies, resected specimens, organ tissues), blood-derived materials (whole blood, plasma, serum, PBMCs), or other bodily fluids (urine, saliva, CSF, synovial fluid). Additional sample types may include CTCs, hair, nails, skin biopsies, bone marrow aspirates, feces, or sputum, depending on availability and the assay requirements.

In some embodiments, the multi-omics data is preprocessed prior to being fed into the machine learning model. This preprocessing step is essential for ensuring that the data are standardized, complete, and optimally structured for robust machine learning analysis regardless of the specific type of genomic instability being assessed, such as MSI, HRD, or other relevant biomarkers. Preprocessing may include normalization of gene expression levels to adjust for technical variability across samples, imputation of missing data to address incomplete measurements, feature selection to reduce dimensionality and focus on the most informative variables, and encoding of categorical variables so they can be effectively used by the model. These preprocessing tasks can be performed using the preprocessing unit 150 and the feature extractor 155 as described with respect to FIG. 1.

The feature set used for analysis can be aligned with those identified as most predictive during the training of the machine learning model, which may include gene expression markers, genomic alterations such as SNVs, indels, CNVs, TMB, immune signatures, and/or relevant clinical features (e.g., patient demographics, tumor type, stage, or prior treatment history). If some required features are missing from a sample, for example due to technical limitations, failed assays, or insufficient sample quantity, imputation techniques are applied to estimate the missing values. These imputation methods may include statistical approaches such as mean, median, or mode substitution for numerical or categorical variables, k-nearest neighbors imputation that leverages information from similar samples, or advanced techniques like multivariate imputation by chained equations (MICE) that account for correlations among features. For example, imputing missing genomic alteration data for a gene in the first set of genes or missing expression data for an immune genes in the second set of immune genes can be performed using the DNA sequencing data, the RNA sequencing data, or available genomic alteration data or available expression data from other genes in the rest of the first set of genes or the second set of immune genes. By applying imputation, the model can generate predictions for all samples, even when the input data is incomplete, thus avoiding the need to repeat costly or time-consuming laboratory tests, recollect samples, or exclude valuable cases from analysis.

The multi-omics data may be tailored to include genomic alteration data for the first set of genes, which may comprise cancer driver genes, DNA repair genes, or additional immune-related or non-immune-related genes. The multi-omics data may also include expression data for the second set of immune genes, such as cytokines, chemokines, TCR or BCR signaling components, NK cell activation markers, or immune checkpoint genes including PDCD1, CD274, CTLA4, and LAG3. In some embodiments, the immune gene set includes key regulators of immune response, and the first set of genes may partially or fully overlap with the immune gene set, depending on the biological pathways of interest. The genomic alteration data includes or can be used to identify alterations such as SNVs, indels, CNVs, gene fusions, splice variants, TMB, and MSI at relevant loci.

In some embodiments, the precise set of features included in the multi-omics data can be determined based on the features selected and validated during the training of the machine learning model (used at block 220). For example, if the model was trained using a specific panel of gene expression markers and genomic alterations identified through feature selection algorithms, the same markers and alterations would be included in the multi-omics data for new subjects to ensure consistency and optimal predictive performance. The composition of the multi-omics panel may be further guided by prior studies, pathway involvement, or predictive value for therapeutic response, such as response to immune checkpoint inhibitors or DNA repair-targeted therapies.

If some features required by the model are missing from the multi-omics data for a new sample due to assay limitations, sample quality, or technical failure, data imputation techniques may be applied. For example, statistical methods such as mean or median substitution, k-nearest neighbors, or multivariate imputation by chained equations can estimate missing values based on the observed patterns in the available data. This approach ensures that the input data remains compatible with the trained machine learning model and enables robust prediction of genomic instability status, even when not all features can be directly measured for every sample. By aligning the selected features in the multi-omics data with those used during model training and applying imputation where necessary, the workflow consistently provides high-quality and analysis-ready data to support accurate genomic instability prediction. In addition, by using imputation to estimate missing values rather than repeating laboratory tests or requesting additional samples, this method reduces the need for redundant or costly experimental procedures. As a result, the workflow saves both time and resources, allowing for faster turnaround of clinically actionable results and more efficient use of laboratory and healthcare system capacity.

In some embodiments, the second set of immune genes includes at least 10, 20, 30, 40, 50, 60, 70, 80, 90, 100, 200, 300, or 400 genes. In some embodiments, these immune genes may include regulators of immune responses, such as cytokines, chemokines, and their receptors, as well as genes involved in antigen presentation, immune cell activation, and checkpoint regulation. Examples of such genes may include encoding interferons (e.g., IFNG, IFNA1), interleukins (e.g., IL6, IL10, IL12A), tumor necrosis factors (e.g., TNF, LTA), chemokines (e.g., CCL2, CXCL10), genes involved in immune cell-specific pathways, such as T-cell receptor (TCR) signaling (e.g., CD3D, CD8A, ZAP70), B-cell receptor (BCR) signaling (e.g., CD19, CD79A, BLNK), and natural killer (NK) cell activation (e.g., KLRK1, PRF1, GZMB), and/or immune checkpoint genes such as PDCD1 (PD-1), CD274 (PD-L1), CTLA4, and LAG3. In some embodiments, the second set of immune genes include RORC, PTK7, TNF, CD276, IDO1, TFRC, LYZ, LRG1, MPO, CCL18, NCAM1, IKZF4, CD8B, IL23A, IL10, CXCL10, CXCL9, TNFSF13B, CD83, CXCL11, CD40, CCL22, IL2RA, IL22, VEGFA, IL17A, and IL15. In some embodiments, the second set of immune genes is selected based on their differential expression patterns observed in prior studies, their involvement in immune-related pathways, or their predictive value for therapeutic response, such as response to immune checkpoint inhibitors or adoptive cell therapies.

In some embodiments, the first set of genes includes both immune-related genes and non-immune-related genes. The first set of genes may include at least 10, 20, 30, 40, 50, 60, 70, 80, 90, 100, 200, 300, or 400 genes. In some embodiments, the first set of genes includes only non-immune-related genes, for example, one or more of TP53, PIK3CA, CDH1, GATA3, PVR, FOXMI, BRCA1, CHD2, MTOR, TBX3, RANBP2, EPHB1, MELK, MAP3K1, BUB1, CDKN2A, BCL2, TUBB, LAMP3, MAGEA10, CCNB2, GBP1, CD47, CXCL1, LCN2, GAGE12J, CDKN3, MAGEA4, TRIM29, ADGRE5, ZEB1, TLR9, RORC, PTK7, PTEN, TBP, ABCF1, FUT4, TNF, CD276, IDO1, TFRC, LYZ, LRG1, MPO, MAD2L1, CCL18, NCAM1, IKZF4, CD8B, RB1, IL23A, IL10, CXCL10, KLF2, M6PR, CXCL9, TNFSF13B, CD83, CXCL11, CDK1, CD40, CCL22, IL2RA, IL22, VEGFA, IL17A, and IL15. In some embodiments, the first set of genes further include the immune genes in the second set. Genomic profiling data, including genomic alterations such as mutations, copy number variations, and structural rearrangements, is identified at locations of the first set of genes. In some embodiments, the genomic alterations include single nucleotide variants (SNVs), insertions and deletions (indels), copy number variations (CNVs), gene fusions, splice variants, tumor mutational burden (TMB), and/or microsatellite instability (MSI).

At block 220, the multi-omics data obtained at block 215 is input into a tree-based machine learning model configured to analyze one or more features by traversing a path from a root node to a terminal node in each tree of the tree-based architecture based on values of one or more features generated from the multi-omics data. The machine learning model may use architectures such as random forest (RF), recursive partitioning and regression trees (rpart), classification and regression trees, or other ensemble tree-based approaches. These models are designed to analyze the complex and high-dimensional relationships present in multi-omics datasets.

For example, in the case of an RF model, the architecture consists of a collection of individual decision trees. Each tree can be trained on a bootstrapped subset of the training data and selects a random subset of features at each split. For each input sample, the RF model evaluates the values of the selected features by traversing from the root node through a hierarchy of decision nodes, where each node applies a threshold to a specific feature derived from the multi-omics data. The sample is routed through the tree depending on whether the feature value satisfies the splitting criterion at each decision point, continuing this process until it reaches a terminal node. Each terminal node assigns a classification or probability score, such as MSI-high, MSS, HRD-high, or HRD-low. The RF model then aggregates the predictions from all trees, for example, by majority voting for classification or averaging for regression, to generate a final robust prediction for the sample.

Similarly, in an rpart model, a single decision tree is constructed by recursively partitioning the data based on values of selected features, with each split chosen to maximize the homogeneity of the outcome variable within the resulting nodes. The rpart model processes the input sample by starting at the root node and following a series of splits determined by the feature values, ultimately arriving at a terminal node that provides the predicted class or probability score for genomic instability status.

The tree-based machine learning models, including RF and rpart, offer significant technical advantages for multi-omics data analysis. These models efficiently process large numbers of heterogeneous input features by focusing computation on only the most informative variables identified during training, often selected using algorithms such as Boruta or importance metrics from random forest analysis. This targeted feature selection reduces computational complexity, minimizes memory requirements, and streamlines the input set, which allows the models to handle high-dimensional datasets while avoiding unnecessary data storage or the use of large parameter matrices. RF and rpart models are able to generate predictions using the available features for each sample, so if certain data points are missing or incomplete, the model still delivers reliable results without requiring imputation or discarding valuable samples. This capacity reduces the need for repeat laboratory testing, saves time by enabling immediate analysis, and lowers costs by eliminating redundant experiments. In addition, the internal structure of these models, which stores only split rules and terminal node outcomes, results in lower memory consumption compared to models that rely on dense parameter storage. An additional benefit is interpretability, as tree-based models provide feature importance rankings that clearly indicate which molecular or clinical features most influenced a given prediction. This transparency supports clinical trust and facilitates review or validation of model outputs. By combining computational and memory efficiency, accurate prediction with incomplete data, and interpretable results, tree-based models enable robust and scalable genomic instability assessment in both research and clinical environments.

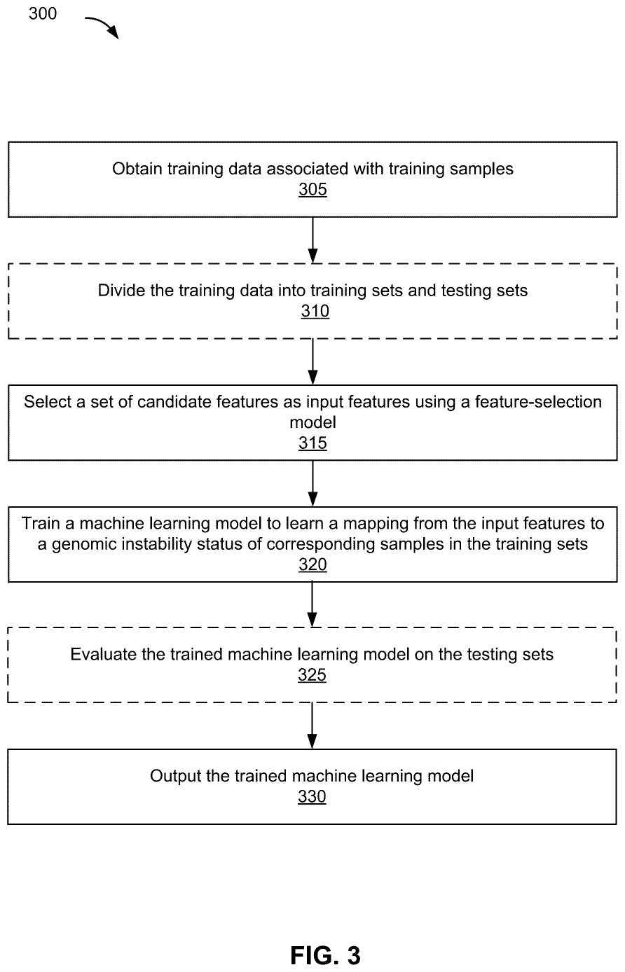

In some embodiments, the machine learning model is trained using training data obtained from training samples. The training samples may be biological samples obtained from patients having been diagnosed with cancer and with known genomic instability statuses. For example, the training samples may be obtained from ovarian cancer patients, from breast cancer patients, or a mix thereof. The training data includes gene expression data associated with a plurality of genes, genomic alteration data, and genomic instability data (e.g., HRD scores). Details regarding training is further illustrated in FIG. 3 and described below.

At block 225, the tree-based machine learning model is used to predict a genomic instability status based on the obtained multi-omics data and/or the values of the selected input features. As discussed above, the tree-based machine learning model may analyze the data by traversing decision paths within each tree of the architecture. The prediction process begins at the root node of each decision tree, where the model examines a specific feature and applies a decision criterion to determine which branch to follow. This evaluation continues through a sequence of internal nodes, each considering a different feature, until the sample arrives at a terminal node that provides an outcome. The outcome at each terminal node represents a classification or probability score related to the genomic instability status. In the case of an ensemble model such as a random forest, multiple decision trees independently process the same input data. Each tree generates its own prediction, and the model aggregates these predictions, commonly by majority vote for a classification task or by averaging for a probability score. The prediction of the subject's genomic instability status may include designations such as MSI-high, MSS, HRD-high, or HRD-low, depending on the specific molecular profile and the model's learned decision logic.

In some embodiments, an MSI status is detected for the subject based on the multi-omics data using the trained machine learning model. The MSI status can be selected from MSI-high or MSS. The machine learning model, after processing the multi-omics features, may generate a probability score that reflects the likelihood of the subject being MSI-high. Additionally or alternatively, the model can produce a numerical MSI score that quantifies the extent of microsatellite instability in the sample. This MSI score may be derived from the integrated analysis of gene expression signatures, genomic alterations such as SNVs, indels, TMB, or direct instability metrics from microsatellite regions, as captured in the multi-omics panel. The predicted MSI status and associated probability or score can be used to provide a comprehensive assessment of the tumor's genomic instability and to guide further clinical decision-making. For example, a classification of MSI-high may identify subjects who are likely to benefit from immune checkpoint inhibitor therapies, while an MSS status may suggest alternative therapeutic options or indicate the need for additional diagnostic testing. In some embodiments, the machine learning model is further trained or configured to provide recommendations regarding the subject's potential response to immunotherapy or to suggest eligibility for molecularly stratified clinical trials, based on the predicted MSI status and the broader molecular profile.

In some embodiments, an HRD status is detected for the subject based on the multi-omics data using the trained machine learning model. The HRD status can be selected from HRD-positive and HRD-negative, or from HRD-high or HRD-low. A probability score indicating the likelihood of HRD and/or a numerical HRD score that quantifies the extent of HRD may also be provided at block 225. For example, the trained machine learning model may generate a probability score that reflects the likelihood of the subject being HRD-positive. Additionally or alternatively, a numerical HRD score that quantifies the extent of the HRD may also be computed. This HRD score can be integrated with other molecular features, such as large-scale genomic events (e.g., loss of heterozygosity, telomeric allelic imbalance, or large-scale state transitions) to provide a comprehensive assessment of the HRD and/or determination of therapeutic strategies (whether to administer PARP inhibitors or platinum-based chemotherapy, or administer alternative treatment regimens).

Optionally, at block 230, the predicted genomic instability status generated by the tree-based machine learning model is provided or communicated to downstream systems, end users, or external devices. In some embodiments, this may be accomplished by generating a testing report that summarizes the findings in a standardized digital or printable format. The testing report can include the initial genomic instability indication determined at block 205 or 210, the predicted genomic instability status determined at block 225, supporting probability or confidence scores, and a list of the most important features that contributed to the model's decision for the individual sample.

In some embodiments, the report includes data visualizations such as feature importance plots, heatmaps, scatter plots, or bar charts to graphically represent key molecular features contributing to the genomic instability status. These visual tools are designed to support intuitive understanding and interpretation of complex multi-omics data by clinicians, laboratory professionals, or researchers. For instance, the report may highlight specific genomic alterations, such as mutations, CNVs, LOH events, or telomeric allelic imbalances, as well as transcriptomic insights like the differential expression of genes associated with DNA repair pathways, immune response, or inflammation. The visualization of these features allows users to quickly identify molecular drivers of the predicted status, whether it be MSI-high, MSS, HRD-high, HRD-low, or any other genomic instability classification produced by the machine learning model.

The report may also include clinical implications based on the predicted genomic instability status, offering recommendations for potential therapeutic strategies or next steps. For example, the report could indicate that a subject classified as MSI-high may be a candidate for immune checkpoint inhibitor therapy, while a subject with HRD-high may benefit from PARP inhibitors or platinum-based chemotherapy. Conversely, for MSS or HRD-low results, the report may suggest consideration of alternative treatment options, further diagnostic workup, or additional molecular testing.

Beyond reporting for human review, the predicted genomic instability status and supporting information can be transmitted electronically to downstream clinical information systems, laboratory information management systems (LIMS), or decision support platforms. The output may be integrated into automated clinical workflows, where it can trigger subsequent technical actions, such as flagging samples for confirmatory testing, generating alerts for pathologists or treating physicians, or adjusting laboratory resource allocation. In research or pharmaceutical settings, the results can be programmatically incorporated into bioinformatics pipelines, used for automated cohort stratification, or drive adaptive trial randomization in clinical studies.

Confirmatory tests may be performed using the same biological sample used in previous workflow steps or, if necessary, using a different sample obtained from the same subject. In some embodiments, the confirmatory testing comprises one or more of the following: (a) MLH1 promoter methylation analysis, which is used to distinguish between sporadic and hereditary causes of MSI; (b) mismatch repair gene sequencing, which can detect pathogenic variants in genes such as MLH1, MSH2, MSH6, or PMS2; (c) loss of heterozygosity (LOH) analysis, which provides additional evidence for HRD status; (d) functional DNA repair assays, which directly assess the cell's ability to repair DNA damage and support the classification of HRD; (e) fluorescence in situ hybridization (FISH) assays, which are used to detect chromosomal rearrangements or copy number changes supporting the initial prediction of genomic instability; (f) chromosomal microarray analysis, which can identify broad genomic alterations such as large deletions, duplications, or copy number variants; (g) targeted sequencing using an extended genomic instability panel, which enables comprehensive assessment of multiple relevant genes or loci; (h) Sanger sequencing, which may be performed to validate specific variants identified by high-throughput sequencing; and (i) immunohistochemistry for mismatch repair proteins, which assesses the expression of key DNA repair proteins if this has not already been evaluated. The selection of specific confirmatory tests is guided by the clinical context, the molecular findings from the initial prediction, and the requirements of downstream clinical or research workflows.

Additional technical applications include leveraging the testing report or model output as an input to automated therapy selection algorithms, where the predicted genomic instability status (for example, MSI-high or HRD-high) can be automatically matched to relevant FDA-approved therapies or clinical guidelines in an electronic decision support system. For instance, a predicted MSI-high status could trigger a recommendation for immune checkpoint inhibitor therapy, while an HRD-high result may prompt the suggestion of PARP inhibitor treatment. The model output can also be used by eligibility assessment tools to determine whether a patient qualifies for targeted therapies or biomarker-driven clinical trials, such as an electronic system that cross-references the predicted status with ongoing trial inclusion criteria and alerts research coordinators to potential matches. In addition, the predicted genomic instability status can be incorporated into electronic systems that guide enrollment in molecularly stratified clinical trials, where patient assignment to trial arms is dynamically managed based on molecular features.

The model output may serve as a digital input for quality assurance modules, which automatically monitor the consistency and performance of genomic testing across a laboratory network. For example, repeated inconclusive results or outlier predictions can trigger automated review or recalibration of laboratory instruments. The results can also be logged in regulatory compliance systems to demonstrate adherence to reporting requirements or in audit trails that document each step of the computational workflow for technical validation and reproducibility.

By providing actionable and machine-readable output in a timely and interpretable format, the workflow 200 supports not only informed clinical decision-making but also enables a range of automated, technical downstream processes. These include but are not limited to integration with electronic health records, triggering of further diagnostic or laboratory workflows, automated population of clinical registries, and support for technical audit and compliance systems. The combination of a structured report, digital interoperability, and actionable output ensures the solution delivers both clinical and technical value, enhances the quality and efficiency of patient care, and supports the broader digital infrastructure required for precision medicine and advanced laboratory automation.

### IV. TRAINING GENOMIC INSTABILITY PREDICTION WORKFLOW

FIG. 3 shows an exemplary workflow 300 for training a machine learning model to genomic instability status using multi-omics data in accordance with embodiments. The processing depicted in FIG. 3 may be implemented in software (e.g., code, instructions, program) executed by one or more processing units (e.g., processors, cores) of the respective systems, hardware, or combinations thereof (e.g., the intelligent selection machine). The software may be stored on a non-transitory store medium (e.g., on a memory device). The workflow 300 presented in FIG. 3 and described below is intended to be illustrative and non-limiting. Although FIG. 3 depicts the various processing steps occurring in a particular sequence or order, this is not intended to be limiting. In certain alternative embodiments, the steps may be performed in some different orders, or some steps may also be performed in parallel.

At block 305, the workflow begins by obtaining training data associated with training samples. The training data may include multi-omics measurements, such as genomic alteration data, gene expression profiles, immunophenotypic features, and relevant clinical or phenotypic annotations (e.g., known genomic instability statuses). The training data can be collected from a variety of sources to ensure a robust and diverse training set. In some embodiments, the training data may be generated from cohort samples collected in-house, for example through sequencing or molecular assays (e.g., the one or more multi-analyte assays) performed on normal and cancer patient samples using the CGIP platform 145. In some embodiments, the cancer is one or more cancer selected from the group consisting of: colorectal cancer, colon adenocarcinoma, rectum adenocarcinoma, uterine cancer, adrenal gland cancer, bile duct cancer, bladder cancer, bone cancer, bone marrow and blood cancer, brain cancer, breast cancer, cervix cancer, esophageal cancer, eye cancer, head and neck cancer, kidney cancer, liver cancer, lung cancer, lymph node cancer, nervous system cancer, ovarian cancer, pancreatic cancer, pleura cancer, prostate cancer, skin cancer, soft tissue cancer, stomach cancer, testicular cancer, thymus cancer, thyroid cancer, and uterine cancer.

In some embodiments, the training data may be obtained from external sources, such as public repositories or commercial databases. Publicly available databases, including TCGA, GEO, and similar resources, provide access to large-scale, well-annotated multi-omics datasets that include transcriptomic profiles, mutation and variant data, genomic instability metrics, and comprehensive clinical annotations for various cancer types. Commercial sources, such as proprietary panels or curated datasets, may also be used to supplement or enhance the training data. Regardless of the source, the training samples must have known genomic instability statuses, such as MSI-high, MSS, HRD-high, or HRD-low, as determined by validated laboratory assays, clinical reports, or reference standards.

In some embodiments, the training data includes (i) gene expression data associated with a plurality of genes, which provides quantitative measurements of transcriptional activity, enabling the identification of functional patterns and regulatory mechanisms that may correlate with HRD status; (ii) genomic alteration data, capturing detailed structural and sequence-level changes within the genome, such as single nucleotide variations, insertions, deletions, copy number variations, and chromosomal rearrangements, and (iii) genomic instability scores for each training sample, which serves as the ground-truth output label during the training phase. The gene expression and genomic alteration data may form the input features for the machine learning model to detect patterns indicative of the genomic instability.

Optionally, at block 310, the training data are divided into training sets and testing sets to enable robust evaluation of the machine learning model's performance. This division can be performed using a variety of methods, each designed to ensure that the resulting subsets are representative of the overall dataset and maintain the distribution of key variables, such as genomic instability status and relevant clinical features.

For example, a simple random split may be applied at block 310, where the training data are randomly assigned to training and testing sets in proportions such as seventy percent (70%) for training and thirty percent (30%) for testing. To address potential imbalances in the distribution of genomic instability statuses (e.g., MSI-high, MSS, HRD-high, and HRD-low), stratified sampling can be used. In stratified sampling, the training data are split so that each subset preserves the proportion of each class or category found in the original dataset, reducing the risk of bias and ensuring fair model evaluation across all groups.

In some embodiments, the training data are partitioned into k approximately equal-sized folds or subsets. For instance, in a ten-fold cross-validation, the data are split into ten folds. The model is then trained on nine folds and evaluated on the remaining fold, and this process is repeated ten times, with each fold serving as the testing set once. The performance metrics from each iteration can be averaged to provide a comprehensive estimate of the model's generalizability and stability. This approach maximizes the use of available data, reduces overfitting, and provides a more reliable assessment of how the model will perform on new, unseen samples.

Other data segmentation methods, such as leave-one-out cross-validation, can also be used when the dataset is particularly small or when maximizing the use of every available sample is important. The choice of splitting method can be informed by the size of the dataset, the distribution of outcome variables, and the intended clinical or research application.

In some embodiments, k-fold cross-validation can iteratively partition the training sets into different combinations of training and validation parts to ensure robust evaluation and reduce the risk of performance variability caused by the specific choice of training-validation splits. By incorporating this additional layer of division, the training process becomes more rigorous and effective, ultimately resulting in a more reliable and generalizable machine learning model. The value of k can range from 2 to 10, or more than 10.

At block 315, a set of candidate features is selected as input features using a feature-selection model. This step contributes to the optimization of the machine learning model's performance by ensuring that the input data are both informative and manageable in size. Feature selection may be performed separately for each data type, such as by first extracting and evaluating features from gene expression data and then independently selecting features from genomic alteration data (or vice versa), including SNVs, indels, CNVs, gene fusions, or other relevant molecular measurements. In some workflows, feature selection may also be performed jointly across the full multi-omics dataset to identify combinations of features that together best predict genomic instability status.

Several algorithms may be employed for this purpose. For example, the Boruta algorithm can be used to assess the importance of each original feature by introducing shadow features, which are random permutations of the data, and comparing the original feature's importance score to that of the shadow features. Features that show a statistically significant contribution are retained as candidate predictors. Similarly, random forest models can provide feature importance metrics based on how much each feature contributes to reducing impurity or classification error within the trees. Additional methods, such as recursive feature elimination or mutual information analysis, may also be used to refine the list of features. In some embodiments, the feature-selection model is pretrained before the training of the machine learning model.

In some embodiments, the feature selection process begins with the generation of shadow features by shuffling the values of each original feature. This ensures that these randomized features do not carry any predictive information. A random forest model is then trained on the dataset, which now includes both original and shadow features, and the importance of all features is calculated. Next, the algorithm compares the importance of each original feature to the maximum importance of the shadow features. Based on this comparison, features are classified as important if their importance is significantly higher than that of the shadow features, unimportant if their importance is significantly lower, and tentative if their importance falls in between. Features classified as unimportant are removed from the dataset, while tentative features are retained for further evaluation in subsequent iterations. This process is repeated until all features are classified as either important or unimportant. The final output of the algorithm is a list of confirmed important features that can be used to train predictive models with improved accuracy and reduced complexity. In some embodiments, more than 400 features are input into the feature-selection model and less than 100 features are selected. In some embodiments, the feature-selection model selects features of the first set of immune genes and features of the second set of genes. For example, expression of 59 immune genes and genomic alterations in 10 genes are selected by the feature-selection model for training the machine learning model to predict the genomic instability status.

The selected candidate features may include a wide range of genomic, transcriptomic, and immunophenotypic variables, as well as composite or derived parameters like TMB or immune gene expression signatures. By focusing on only the most informative features, the feature selection process reduces computational complexity, allows the model to process high-dimensional data more efficiently, and minimizes the risk of overfitting. To ensure the robustness and stability of the selected features, validation techniques such as cross-validation within the training data are applied, confirming that the chosen feature set consistently supports accurate prediction of genomic instability status across different subsets of the data. This targeted and validated feature selection improves both the accuracy and interpretability of the final machine learning model, supporting its application in clinical and research settings. In some embodiments, the candidate features are treated as a hyperparameter of the machine learning model and may be adjusted through cross validation or other validation or hyperparameter adjustment approaches.

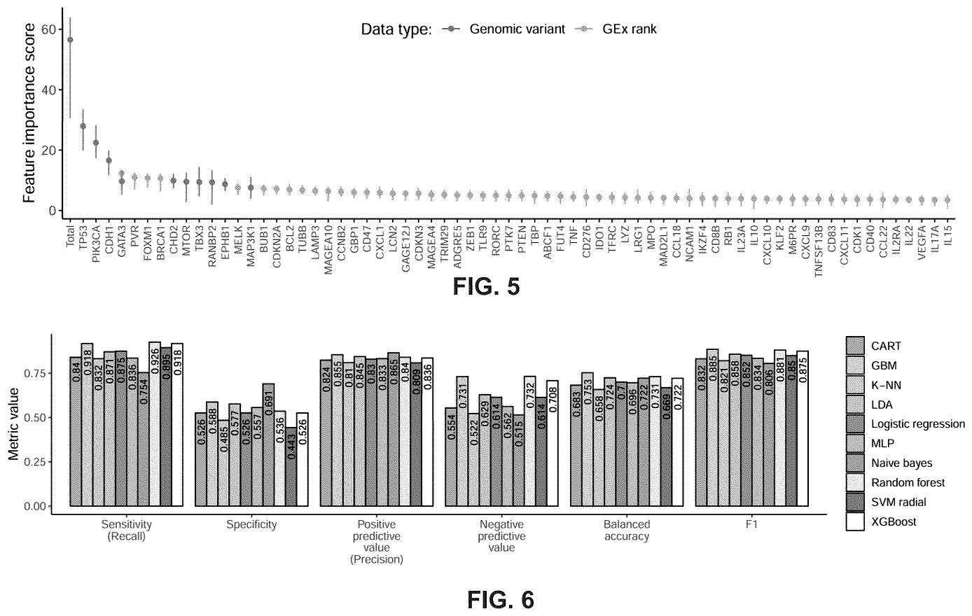

For example, a set of HRD-related features may be selected to serve as input features for the machine learning model. This step begins with obtaining a broader set of features derived from the training data, which may include various biological attributes such as gene expression levels, genomic alteration metrics, or immune-related markers. Using a feature-selection model, such as the Boruta algorithm or Random Forest, the most relevant predictors of HRD status can be identified from this broader feature set. These feature-selection models evaluate the importance of each feature based on statistical significance or contribution to predictive accuracy, retaining only those features that are highly correlated with HRD status. For instance, the process may identify features such as the total number of genomic alterations or key expression patterns in immune-related genes. By narrowing the feature set to only the most informative variables, this step reduces computational complexity and enhances the accuracy of the machine learning model. For example, FIG. 5 shows feature importance scores generated using the Boruta algorithm for various genomic and gene expression (GEx) factors, ranked by their predictive significance. The x-axis lists specific genes or genetic features, while the y-axis represents their corresponding importance scores. Two data types are represented—genomic variants (dark blue) and GEx ranks (light blue)—with error bars indicating variability or uncertainty in the scores.

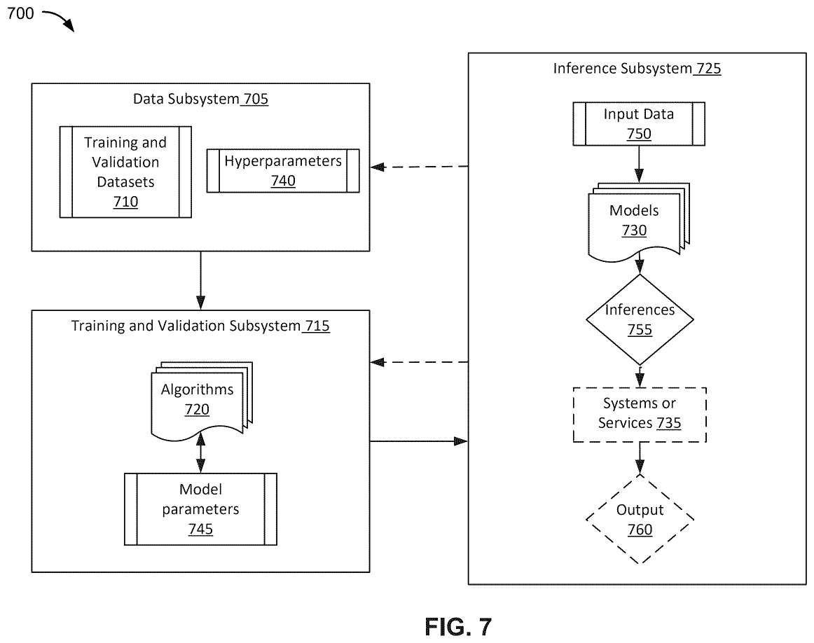

At block 320, a machine learning model is trained to learn a mapping from the input features to the genomic instability status of the corresponding samples in the training sets. The model architecture may include tree-based models such as random forest or rpart, gradient boosting models, support vector machines, or neural networks. Each of these model types can be implemented and managed by the training and validation subsystem 715 described in FIG. 7, which provides the computational resources and workflow management necessary for robust model development.

During the training process, the selected input features, such as gene expression markers, genomic alterations like SNVs, indels, CNVs, TMB, immune-related features, and relevant clinical variables, are supplied to the model along with the known genomic instability statuses of the training samples. The model learns associations between these features and the corresponding outcomes by adjusting its internal parameters to minimize prediction error, typically measured by a loss function suited to the classification or regression task at hand.

During the training, the model iteratively updates its internal weights or decision rules to align with the ground-truth labels provided in the training data. The validation parts, on the other hand, are held out from the direct training process and are used to evaluate the model's performance during intermediate stages of training. This division serves as an internal checkpoint, enabling the monitoring of the model's ability to generalize to unseen data. By assessing the model on the validation parts, issues like overfitting or underfitting can be detected early. The validation parts help strike a balance by providing feedback to guide hyperparameter tuning, such as adjusting the learning rate, model complexity, or regularization parameters. In some embodiments, early stopping techniques are adopted to monitor the model's performance on the validation parts and halts training when performance ceases to improve, thereby preventing overfitting. Moreover, the use of validation parts supports model selection by allowing the comparison of different model architectures or configurations to identify the one that performs best on the validation data.

For tree-based models like random forest or rpart, the algorithm constructs decision trees by recursively partitioning the data based on values of selected features. Each split is chosen to maximize the homogeneity of the genomic instability status within the resulting nodes, enabling the model to capture complex relationships between features such as gene expression levels, SNVs, indels, CNVs, TMB, and clinical or immunophenotypic variables. In the case of a random forest, the architecture consists of many individual decision trees, each trained on a bootstrapped subset of the training data and using a random subset of features at each split. This ensemble approach increases robustness, provides better generalization to new data, and reduces overfitting by averaging the predictions from multiple independent trees.

A significant technical benefit of using tree-based models, including random forest and rpart, is their ability to efficiently process large numbers of heterogeneous input variables while minimizing computational and memory costs. These models do not require storing large parameter matrices or performing complex matrix operations, as is common in neural networks or some regression-based methods. Instead, they store only the split rules and terminal node outcomes, which significantly reduces memory usage and enables fast inference. This memory efficiency allows for the deployment of the models in resource-limited clinical or laboratory environments and supports high-throughput batch processing of multi-omics samples.

Tree-based models also have the advantage of being able to generate predictions using only the available features for each sample. If certain data points are missing or incomplete, the model can still produce reliable results without discarding the sample or requiring computationally expensive imputation in all cases. This feature is particularly valuable in real-world clinical settings, where incomplete data are common and repeating laboratory assays may not be feasible due to time or cost constraints.

In some embodiments, during training, feature selection is integrated into the tree construction process. Features that provide the most information about genomic instability status are prioritized, and features that do not contribute meaningfully are excluded, further reducing computational burden and memory requirements. Algorithms such as Boruta or feature importance metrics from random forest analysis can be used to formally select the best subset of features during model training. The Boruta algorithm operates as a wrapper method around random forest, iteratively comparing the importance of each actual feature to the importance of randomly permuted shadow features. In each iteration, Boruta adds shadow features created by shuffling the values of the original features, then fits a random forest to the dataset containing both original and shadow features. The algorithm calculates importance scores for all features and classifies them as important, unimportant, or tentative by comparing their scores to the maximum score achieved by any shadow feature. Features that consistently outperform the shadow features are retained as important, while those that do not are removed. This process is repeated until all features are classified, yielding a final subset of informative features that have demonstrated a statistically significant contribution to the prediction task.

Similarly, feature importance metrics derived from random forest analysis can be used to rank input features by their influence on model accuracy or node impurity. For example, the mean decrease in Gini impurity or the mean decrease in accuracy when a feature is permuted can be calculated for each feature in the dataset. Features with the highest importance scores are selected for further modeling, while less informative features are excluded to reduce dimensionality and computational overhead. This selection of features enhances predictive accuracy, reduces the risk of overfitting, streamlines model complexity, and supports interpretability by highlighting which features are most critical for determining genomic instability status. The selected features are further validated for stability and relevance by cross-validation within the training data, ensuring that the final model generalizes well to new, unseen cases in both clinical and research applications.

Throughout training, hyperparameter tuning and regularization techniques may be employed to further optimize model performance. Hyperparameters such as the number of trees in a random forest, the maximum depth of trees, the learning rate for gradient boosting, or the architecture of a neural network are systematically adjusted, often using grid search or random search strategies. Regularization methods, such as limiting tree depth, early stopping, or adding penalty terms, are applied to prevent overfitting and ensure the model generalizes well to new data.

The training process may also include monitoring performance metrics such as accuracy, sensitivity, specificity, F1 score, and ROC-AUC on validation data to guide model selection and optimization. Once the model's parameters are optimized and its performance is validated, the trained machine learning model is ready for further evaluation or deployment, as depicted in the subsequent blocks of the workflow in FIG. 3, and is integrated with the prediction and reporting systems shown in FIG. 2 and FIG. 7. This comprehensive training approach ensures that the resulting model can accurately predict genomic instability status based on complex, high-dimensional multi-omics input data.

Optionally, at block 325, the trained machine learning model is evaluated on the testing sets to assess its ability to generalize to new, unseen data. Evaluation employs one or more performance metrics that reflect different aspects of predictive accuracy and clinical relevance. These metrics may include sensitivity, which measures the proportion of true positive cases correctly identified; specificity, which quantifies the proportion of true negatives accurately predicted; positive predictive value and negative predictive value, which provide information on the reliability of positive and negative predictions; balanced accuracy, which accounts for class imbalance; F1 score, which combines precision and recall into a single measure; and area under the receiver operating characteristic curve (ROC-AUC), which summarizes the model's ability to discriminate between classes across various threshold settings.

To further support robust evaluation, metrics such as the area under the precision-recall curve (PR-AUC) may be used, especially when the prevalence of genomic instability is low or when the model is deployed in a screening context. The evaluation may be conducted using dedicated modules within the genomic instability prediction platform 115, which can automate metric calculation and support visualization of confusion matrices, ROC curves, and feature importance plots. The results of the model evaluation can be reviewed by system administrators, clinical researchers, or data scientists via the client device 105, enabling real-time or retrospective validation of model performance.

In some embodiments, supervised learning algorithms such as Classification and Regression Trees (CART), Gradient Boosting Machine (GBM), K-Nearest Neighbors (K-NN), Linear Discriminant Analysis (LDA), Logistic Regression, Multi-Layer Perceptron (MLP), Naive Bayes, Random Forest, Support Vector Machine with Radial Basis Function Kernel (SVM Radial), and/or Extreme Gradient Boosting (XGBoost) may be used. The choice of algorithm depends on the nature of the dataset, the complexity of the problem, and the desired trade-offs between accuracy, interpretability, and computational efficiency. In some embodiments, the training is unsupervised or semi-supervised training.

In some embodiments, a plurality of machine learning algorithms may be selected to train a plurality of machine learning models. For instance, the selected algorithms may include CART, GBM, K-NN, LDA, Logistic Regression, MLP, Naïve Bayes, Random Forest, SVM Radial, and XGBoost. A machine learning model is trained for each algorithm in the plurality of selected algorithms. Training involves fitting each algorithm to the provided dataset, allowing it to learn patterns, relationships, and rules that map the input features to the target variable. During this phase, the model's parameters are optimized to minimize the prediction error, and specific techniques such as hyperparameter tuning may be applied to further refine the training process. After training is complete for all models, one or more performance metrics are generated for each trained machine learning model. Performance metrics provide a quantitative assessment of how well each model has learned from the training data and how effectively it generalizes to unseen data. Metrics such as accuracy, sensitivity, specificity, positive predictive value (PPV), negative predictive value (NPV), balanced accuracy, F1 score, and Receiver Operating Characteristic-Area Under the Curve (ROC-AUC) are calculated to capture various aspects of the models' performance. Additionally, k-fold cross-validation may be employed to improve the robustness of the performance assessment by averaging the metrics across multiple training and testing splits.

One or more machine learning models may be selected based on the generated performance metrics. This selection process entails comparing the metrics of all trained models to determine which algorithm delivers the best performance for the given problem and dataset. The criteria for selection may vary depending on the specific application and priorities. For instance, in scenarios where minimizing false negatives is critical, such as medical diagnostics, a model with high sensitivity may be preferred. Conversely, for applications like fraud detection, where false positives carry a high cost, specificity or precision might take precedence. The selected model represents the optimal balance between performance, interpretability, and computational efficiency, tailored to the problem's constraints and objectives. By following this systematic approach of algorithm selection, training, evaluation, and comparison, the process ensures that the most suitable machine learning model is chosen for deployment, enhancing the reliability and effectiveness of the solution.

FIG. 6 shows performance of various ML algorithms across six metrics: Sensitivity (Recall), Specificity, Positive Predictive Value (Precision), Negative Predictive Value, Balanced Accuracy, and F1 Score. Each bar represents the performance of a specific algorithm, using distinct colors for models such as CART, GBM, K-NN, LDA, Logistic Regression, MLP, Naive Bayes, Random Forest, SVM Radial, and XGBoost. The x-axis represents the metrics, while the y-axis shows scores ranging from 0 to 1. Performance varies across algorithms, with GBM, Random Forest, and XGBoost consistently achieving higher scores across most metrics, highlighting their effectiveness for predictive tasks.

At block 330, the final trained machine learning model is output, optionally along with the one or more performance metrics associated with the final trained machine learning model. This output model may be deployed in a variety of clinical or research environments, as illustrated in FIG. 2 and supported by the environment in FIG. 1. The deployment can include integration into automated prediction workflows, such as those managed by the genomic instability prediction platform 115, or direct use within laboratory information management systems, clinical decision support tools, or research analytics platforms. The trained model is capable of receiving new multi-omics data as input, including data generated from the genomic instability testing platform 102, the CGIP platform 145, or other clinical or laboratory data sources. Upon receiving new multi-omics data, the trained model can provide actionable predictions of genomic instability status for future samples, such as identifying MSI-high, MSS, HRD-high, or HRD-low cases.

Deployment of the model enables automated, real-time analysis of complex multi-omics datasets, supporting timely and accurate determination of genomic instability in patient samples. The model can be configured to generate output in formats compatible with downstream reporting systems, electronic health records, or research databases, facilitating seamless integration into existing clinical and laboratory workflows. Additionally, the model's interpretability features, such as feature importance scores and transparent decision logic, support regulatory compliance, clinical validation, and auditability. The trained model may also be stored for subsequent use, allowing for periodic updates, retraining with new data, or benchmarking against emerging standards and datasets. By making the trained machine learning model readily available for real-world application, the workflow ensures continuous improvement in the accuracy, efficiency, and scalability of genomic instability prediction in both clinical diagnostics and translational research.

### V. COMPREHENSIVE GENOMIC AND IMMUNE PROFILING ASSAYS

### 1. Obtaining Samples

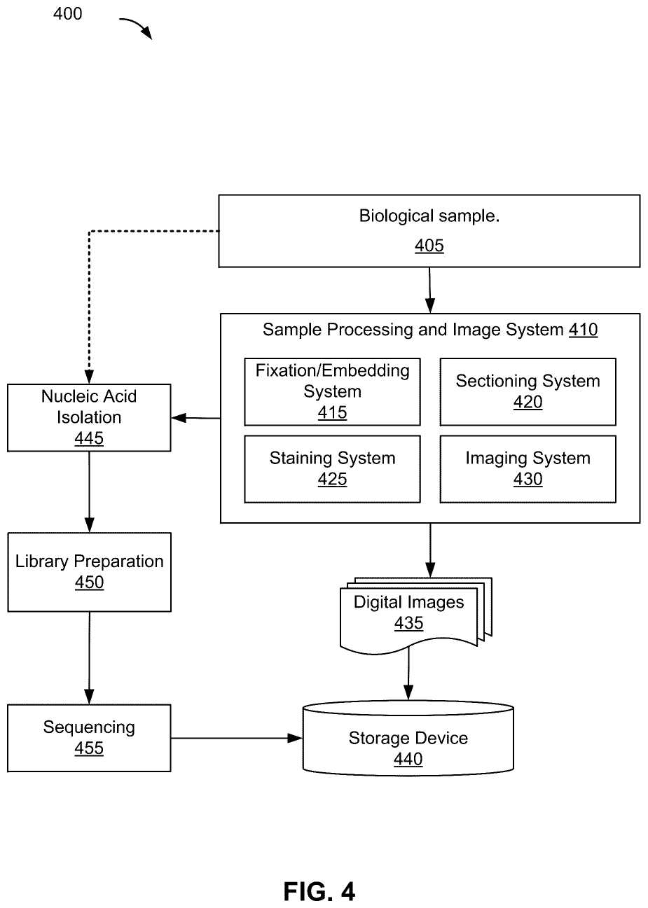

FIG. 4 shows an exemplary system 400 for generating pathology images (e.g., IHC data) and sequencing data for comprehensive genomic and immune profiling. The system 400 can be implemented as software, such as code, instructions, or programs executed by one or more processing units, including processors or cores, as well as hardware components (e.g., imaging sensor) or combinations of hardware and software. The software may reside on a non-transitory storage medium, such as a memory device, to enable persistent and reliable operation. The system 400 presented in FIG. 4 and described below is intended to be illustrative rather than limiting. Although FIG. 4 depicts various processing steps and units occurring in a particular sequence or order, this arrangement is not intended to be restrictive. In some embodiments, the steps may be performed in a different order, or some steps may be executed in parallel to improve efficiency or accommodate specific workflow requirements. In some embodiments, the system 400 depicted in FIG. 4 may be implemented by components within the environment 100 described with respect to FIG. 1.

A biological sample (or a specimen) 405 may be obtained from a patient using a variety of clinical or laboratory procedures. The biological sample 405 can be a liquid or a tissue that comprises nucleic acid molecules (e.g., DNA and RNA). The type of biological sample 405 that may be collected can be diverse and can include, but are not limited to, amniotic fluid, tissue biopsies from solid organs or tumors, peripheral whole blood or blood cells, bone marrow aspirates, fine needle biopsy samples, peritoneal fluid, plasma and serum samples, pleural fluid, saliva and buccal swabs, semen, and tissue homogenates. The biological sample 405 can also include frozen sections of tissue or formalin-fixed, paraffin-embedded (FFPE) tissue sections. Methods of obtaining a biological sample 405 include but are not limited to biofilms, aspirations, tissue sections, swabs, phlebotomy, surgical or needle biopsies, and the like.

The biological sample 405 can be obtained from a wide range of subjects, including healthy volunteers as controls or reference populations, or from subjects with a disease, such as cancer, infectious diseases, autoimmune conditions, or genetic disorders. In oncology, specimens may be collected at diagnosis, during treatment, or at disease progression to support longitudinal monitoring and therapy selection. In other contexts, specimens can be used for early detection, screening, or population-level genomic studies.

Following sample acquisition, the biological sample 405 can enter a sample processing and image system 410 that comprises a fixation/embedding system 415, a sectioning system 420, a staining system 425, and an imaging system 430. Sample processing and image system 410 prepares the biological sample 405 for staining, such as histological staining, to highlight features of interest and enhance contrast in sectioned tissues or cells of the biological sample 405. For example, staining may be used to mark particular types of cells and/or to flag particular types of nucleic acids and/or proteins to aid in the microscopic examination. The stained sample can then be assessed to determine or estimate a quantity of features of interest in the sample (e.g., which may include a count, density or expression level) and/or one or more characteristic of the features of interest (e.g., locations of the features of interest relative to each other or to other features, shape characteristics, etc.).

A fixation/embedding system 415 fixes and/or embeds the sample 405 (e.g., a sample including at least part of at least one tumor). More specifically, a fixation process may be performed for histopathological images, to preserve the sample and slow down sample degradation. In histology, fixation generally refers to an irreversible process of using of chemicals to retain the chemical composition, preserve the natural sample structure, and maintain the cell structure from degradation. Fixation may also harden the cells or tissues for sectioning. Fixatives may enhance the preservation of samples and cells using cross-linking proteins. The fixatives may bind to and cross-link some proteins, and denature other proteins through dehydration, which may harden the tissue and inactivate enzymes that might otherwise degrade the sample. The fixatives may also kill bacteria. The fixatives may be administered, for example, through perfusion and immersion of the tissue sample for a predetermined amount of time. Examples of liquid fixatives include a formaldehyde solution, neutral buffered formalin (NBF) or paraffin-formalin (paraformaldehyde-PFA), methanol, and Bouin. In cases where a sample is a liquid sample (e.g., blood or cells), the sample may be smeared onto a slide and dried prior to fixation. Further, liquid sample processing may also omit embedding and/or sectioning techniques described below.

Embedding may include infiltrating a fixed sample (e.g., a fixed tissue sample) with a suitable histological wax such as a paraffin wax and/or one or more resins, such as styrene or polyethylene). The histological wax may be insoluble in water or alcohol, but may be soluble in a paraffin solvent, such as xylene. Therefore, the water in the tissue may need to be replaced with xylene. To do so, the sample may be dehydrated first by gradually replacing water in the sample with alcohol, which can be achieved by passing the tissue through increasing concentrations of ethyl alcohol (e.g., from 0 to about 100%). After the water is replaced by alcohol, the alcohol may be replaced with xylene, which is miscible with alcohol. Embedding may include embedding the sample in warm paraffin wax. Because the paraffin wax may be soluble in xylene, the melted wax may fill the space that is filled with xylene and was filled with water before. The wax filled sample may be cooled down to form a hardened block that can be clamped into a microtome for section cutting. In some cases, deviation from the above example procedure results in an infiltration of paraffin wax that leads to inhibition of the penetration of antibody, chemical, or other fixatives.

A sectioning system 420 is used to section or slice the fixed and/or embedded tissue sample using, for example, a cryostat, a microtome, a vibratome or compresstome. Sectioning is the process of cutting the tissue sample to obtain a series of sections, with each section having a thickness of, for example, 4-5 microns. In some cases, tissues can be frozen rapidly in dry ice or Isopentane and can then be cut in a refrigerated cabinet (e.g., a cryostat) with a cold knife. Other types of cooling agents can be used to freeze the tissues, such as liquid nitrogen. The sections for use with light microscopy are generally on the order of 4-10 μm thick. In some cases, sections can be embedded in an epoxy or acrylic resin, which may enable thinner sections (e.g., <2 μm) to be cut. The sections may be mounted on glass slides.

Staining system 425 can implement various staining methods to the tissue sections to render relevant structures more visible. In some instances, the staining is performed manually. In some instances, the staining is performed semi-automatically or automatically. Staining can include exposing an individual section of the tissue to one or more different stains (e.g., consecutively or concurrently) to express different characteristics of the tissue. For example, each section may be exposed to a predefined volume of a staining agent for a predefined period of time.

Many staining solutions are aqueous. Thus, to stain tissue sections, the embedding agent (e.g., wax) may need to be dissolved and replaced with water (rehydration) before a staining solution is applied to a section. For example, the section may be sequentially passed through xylene, decreasing concentration of ethyl alcohol (from about 100% to 0%), and water. Once stained, the sections may be dehydrated again and placed in xylene. The section may then be mounted on microscope slides in a mounting medium dissolved in xylene. A coverslip may be placed on top to protect the sample section. The evaporation of xylene around the edges of the coverslip may dry the mounting medium and bond the coverslip firmly to the slide.

Various types of staining protocols may be used to perform the staining. One exemplary type of tissue staining is histochemical staining, which uses one or more chemical dyes (e.g., acidic dyes, basic dyes) to stain tissue structures. Histochemical staining may be used to indicate general aspects of tissue morphology and/or cell microanatomy (e.g., to distinguish cell nuclei from cytoplasm, to indicate lipid droplets, etc.). One example of a histochemical stain is hematoxylin and eosin (H&E). Other examples of histochemical stains include trichrome stains (e.g., Masson's Trichrome), Periodic Acid-Schiff (PAS), silver stains, and iron stains. The molecular weight of a histochemical staining reagent (e.g., dye) is typically about 500 kilodaltons (kD) or less, although some histochemical staining reagents (e.g., Alcian Blue, phosphomolybdic acid (PMA)) may have molecular weights of up to two or three thousand kD. One case of a high-molecular-weight histochemical staining reagent is alpha-amylase (about 55 kD), which may be used to indicate glycogen.

Another type of tissue staining is immunohistochemistry (IHC, also called “immunostaining”), which uses a primary antibody that binds specifically to the target antigen of interest (e.g., a biomarker, a cell lineage marker, a cell surface protein). IHC may be direct or indirect. In direct IHC, the primary antibody is directly conjugated to a label (e.g., a chromophore or fluorophore). In indirect IHC, the primary antibody is first bound to the target antigen, and then a secondary antibody that is conjugated with a label (e.g., a chromophore or fluorophore) is bound to the primary antibody. The molecular weights of IHC reagents are much higher than those of histochemical staining reagents, as the antibodies have molecular weights of about 150 kD or more.

Imaging system 430 scans or generates a raw digital pathology or histopathology digital image of the stained samples. In some instances, each stained tissue section may be mounted on a slide, which is then scanned by to create a digital image. Instances where IHC was performed with one or more chromophore/fluorophore, the stained image may first be visualized using a fluorescent microscope where the image is automatically digitized. A microscope (e.g., an electron or optical microscope) can be used to magnify the stained sample. For example, optical microscopes or electron microscopes may be used. An imaging device (combined with the microscope or separate from the microscope) images the magnified biological sample to obtain the image data. The image data may be a multi-channel image (e.g., a multichannel fluorescent) with several channels. The imaging device may include, without limitation, a camera (e.g., an analog camera, a digital camera, etc.), optics (e.g., one or more lenses, sensor focus lens groups, microscope objectives, etc.), imaging sensors (e.g., a charge-coupled device (CCD), a complimentary metal-oxide semiconductor (CMOS) image sensor, or the like), photographic film, or the like. An image sensor, for example, a CCD sensor can capture a digital image of the biological sample. In some embodiments, the imaging device is a brightfield imaging system, a multispectral imaging system or a fluorescent microscopy system. The imaging device may utilize nonvisible electromagnetic radiation (UV light, for example) or other imaging techniques to capture the image.

Once the biological sample 405 has been processed by the sample processing and imaging system 410, digital images 435 are generated. The digital images 435 may be subsequently examined by digital pathology image analysis and/or interpreted by a human pathologist (e.g., using image viewer software). The pathologist may review and manually annotate the images of the slides (e.g., tissue degeneration, tissue damage, etc.) to enable the use of image analysis algorithms to extract meaningful quantitative measures (e.g., to detect and classify biological objects of interest). Conventionally, the pathologist may manually annotate each successive image of multiple tissue sections from a tissue sample to identify the same aspects on each successive tissue section.

The images of the stained sections may then be stored in a storage device 440, such as a server, a database, or a data repository. The digital images 435 may be stored locally, remotely, and/or in a cloud server. Each digital image 435 may be stored in association with an identifier of a subject and a date (e.g., a date when a sample was collected and/or a date when the image was captured). During analysis, a digital image 435 may further be transmitted to another system (e.g., a system associated with a pathologist, an automated or semi-automated image analysis system, or a machine learning training and deployment system, as described in further detail herein).

In addition to generating a digital pathology image, system 400 can additionally or alternatively be used to prepare the biological sample 405 for CGP, as described in section 2 “Genomic Profiling” below.

### 2. Genomic Profiling and Immune Profiling

CGIP, or comprehensive genomic and immune profiling, is an advanced NGS-based approach that enables the simultaneous detection and characterization of a broad spectrum of genomic alterations and immune-related features across hundreds of genes in a single integrated assay. This CGIP workflow provides a thorough molecular and immunological portrait of a biological sample by interrogating multiple classes of DNA and RNA changes together with immune biomarkers. CGIP can also be employed to identify clinically actionable cancer biomarkers and genomic signatures, such as TMB, HRD, or MSI, as well as immune gene expression profiles and markers of immune activity, each of which can inform diagnosis, therapeutic selection, and patient prognosis. The range of possible genomic alterations detectable by CGIP includes SNVs, which are point mutations in the DNA sequence that may result in missense, nonsense, or silent changes within coding regions, or may affect regulatory elements in non-coding regions. Small insertions and deletions (indels) can also be detected, involving the addition or loss of a small number of nucleotides that can cause frameshift mutations or disrupt gene function. Copy number variations (CNVs) are identified, representing gains or losses of chromosomal segments that lead to amplification or deletion of one or more genes, with significant implications for oncogene activation or tumor suppressor loss. CGIP also captures large structural variants, such as genomic rearrangements including translocations, inversions, duplications, or large deletions, which may disrupt gene integrity or create fusion genes. Splice site alterations and aberrant splicing events are identified when mutations affect the normal splicing of pre-mRNA, potentially resulting in loss of function or the production of abnormal protein products.

In addition to these genomic features, CGIP includes immune profiling components, such as quantification of immune gene expression, assessment of immune cell infiltration markers, and identification of gene expression signatures related to tumor inflammation or immune activity. These immune profiling results may include markers such as PD-L1 expression, T-cell inflamed gene expression profiles, and measures of tumor inflammation or immune evasion. By combining comprehensive genomic and immune data in a single workflow, CGIP provides a more complete understanding of the tumor microenvironment and supports precision oncology strategies that integrate both molecular and immunological insights.

As illustrated in FIG. 4, the CGIP workflow begins with the isolation of nucleic acid molecules at block 445, where DNA, RNA, or both may be isolated. Nucleic acids may be isolated either directly from the biological sample 405, such as tissue or blood, or from an FFPE block generated by the fixation/embedding system 415. In one aspect, DNA is isolated, and the DNA may be genomic DNA, mitochondrial DNA, cell-free DNA (cfDNA), circulating tumor DNA (ctDNA), or a combination thereof. For RNA extraction, total RNA may be isolated from tissues, cells, or body fluids, and can include messenger RNA (mRNA), ribosomal RNA (rRNA), microRNA (miRNA), or other non-coding RNAs, depending on the downstream application. A variety of commercial kits and protocols are available for both DNA and RNA isolation, including those specifically optimized for FFPE tissue, which often require deparaffinization and reversal of crosslinking before enzymatic digestion.

The general process for nucleic acid extraction involves several steps. First, the starting material is subjected to disruption and lysis, which can be accomplished using detergents, hypotonic solutions, proteinase K, chaotropic salts, sonication, or mechanical homogenization. For RNA isolation, additional care is taken to inhibit RNases and preserve RNA integrity, often by including guanidinium thiocyanate or other RNase inhibitors in the lysis buffer. After lysis, proteins and other contaminants are removed, for example, by phenol-chloroform extraction, protease treatment, or column-based purification. The nucleic acids are then separated from the lysate by precipitation with ethanol or isopropanol, binding to a silica membrane in a spin column, or magnetic bead capture, with the choice of method determined by the required purity, yield, and the intended downstream use. For RNA, a DNase digestion step may be included to remove contaminating DNA. Quality and integrity of the extracted DNA or RNA are typically assessed by spectrophotometry, such as using a NanoDrop instrument to determine purity ratios, by fluorometric quantification, and, for RNA, by calculating the RNA integrity number (RIN) using instruments such as an Agilent Bioanalyzer or TapeStation.

Following DNA isolation, the isolated DNA undergoes library preparation 450. Library preparation 450 can be performed using any suitable method known in the art. For DNA, library preparation can be performed using immobilization on a solid phase, enrichment, amplification, cloning, or detection. For RNA, total RNA or mRNA first undergoes reverse transcription to generate complementary DNA (cDNA), which is then used as input for library construction.

A NGS library preparation workflow for DNA includes several steps: first, fragmenting the isolated DNA into a plurality of shorter double stranded DNA target fragments. In general, fragmentation of DNA may be performed physically (e.g., acoustic shearing, sonication, etc.), or enzymatically (e.g., treated with DNase I or other DNA cleavage enzymes). The fragments of DNA can range from 150 to 500 base pairs for short-read sequencing or up to 20 kilobases or more for long-read sequencing. Second, the fragments of DNA are end repaired using an exonuclease and then extend by a single nucleotide (e.g., adenosine (A) for A-tailing) to generate a 3′ overhang on each strand. Then adapter ligation, facilitated by a DNA ligase enzyme, attaches an adapter oligonucleotide sequence using the 3′ overhang. The adapter sequence (e.g., a hairpin, Y-adapter, etc.) can be complementary to flow cell anchors and include a unique molecular identifier sequence. Optionally, the DNA library, or parts thereof, can be amplified (e.g., amplified by a PCR-based method). The prepared DNA library can also be quantified and quality-checked, for example, using qPCR, fluorometric assays, or bioanalyzer analysis.

For RNA library preparation, after reverse transcription to cDNA, the resulting cDNA is fragmented as needed, end repaired, and ligated to sequencing adapters, similar to the process for DNA. Molecular barcodes or indices may be added at this stage to enable multiplexed sequencing. Size selection and purification steps ensure the cDNA fragments are of appropriate length and free of contaminants. PCR amplification is then performed to enrich the library, and the final cDNA library is quantified and quality-checked prior to sequencing. In some workflows, target enrichment methods, such as hybrid capture or amplicon-based selection, may be employed to focus on specific genes or transcripts of interest, including immune-related or cancer biomarker genes.

The prepared DNA or RNA-derived cDNA library is added to a flow cell and immobilized by hybridization to anchors under suitable conditions in preparation for sequencing 455. For RNA sequencing, total RNA or messenger RNA is first reverse transcribed to cDNA, which is then prepared as a sequencing library in a process analogous to that used for DNA, including adapter ligation, size selection, and amplification as needed. Both DNA and RNA libraries can be sequenced on a variety of platforms. Depending on the sequencing platform and workflow, the library can be sequenced to yield either a full or a partial sequence of each fragment. Sequencing platforms may employ a variety of chemistries and detection principles. Any suitable method of sequencing nucleic acids can be used, non-limiting examples of which include Maxim & Gilbert, chain-termination methods, sequencing by synthesis, sequencing by ligation, sequencing by mass spectrometry, microscopy-based techniques, the like or combinations thereof. In some embodiments, a first-generation technology, such as, for example, Sanger sequencing methods including automated Sanger sequencing methods, including microfluidic Sanger sequencing, can be used in a method provided herein. In some embodiments, sequencing technologies that include the use of nucleic acid imaging technologies (e.g., transmission electron microscopy (TEM) and atomic force microscopy (AFM)), can be used. In some embodiments, a high-throughput sequencing method is used. High-throughput sequencing methods generally involve clonally amplified DNA templates or single DNA molecules that are sequenced in a massively parallel fashion, sometimes within a flow cell. Next generation (e.g., 2nd and 3rd generation) sequencing techniques capable of sequencing DNA in a massively parallel fashion can be used for methods described herein and are collectively referred to herein as “massively parallel sequencing” (MPS). In certain embodiments, a non-targeted approach is used where most or all nucleic acids in a sample are sequenced, amplified and/or captured randomly.

Other suitable sequencing technologies may include single molecule, real-time (SMRT) technology of Pacific Biosciences (in SMRT, each of the four DNA bases is attached to one of four different fluorescent dyes. These dyes are phospholinked. A single DNA polymerase is immobilized with a single molecule of template single stranded DNA at the bottom of a zero-mode waveguide (ZMW) where the fluorescent label is excited and produces a fluorescent signal, and the fluorescent tag is cleaved off. Detection of the corresponding fluorescence of the dye indicates which base was incorporated); nanopore sequencing (DNA is passed through a nanopore and each base is determined by changes in current across the pore, as described in Soni & Meller, 2007, Progress toward ultrafast DNA sequence using solid-state nanopores, ClinChem 53 (11): 1996-2001); chemical-sensitive field effect transistor (chemPET) array sequencing (e.g., as described in U.S. Pub. 2009/0026082); and electron microscope sequencing (as described, for example, by Moudrianakis, E. N. and Beer M., in Base sequence determination in nucleic acids with the electron microscope, III. Chemistry and microscopy of guanine-labeled DNA, PNAS 53:564-71 (1965).

In some embodiments, NGS sequencing methods (e.g., whole-genome, exome, targeted, or RNA sequencing (RNA-Seq), etc.) are performed on the prepared DNA or cDNA library samples. NGS is based on the amplification of DNA on a solid surface using fold-back PCR and anchored primers. The prepared DNA library fragments are attached to the surface of flow cell channels are extended and bridge amplified. The fragments become double stranded, and the double stranded molecules are denatured. Multiple cycles of the solid-phase amplification followed by denaturation can create several million clusters of approximately 1,000 copies of single-stranded DNA molecules of the same template in each channel of the flow cell. Primers, DNA polymerase and four fluorophore-labeled, reversibly terminating nucleotides are used to perform sequential sequencing. After nucleotide incorporation, a laser is used to excite the fluorophores, an image is captured, and the identity of the first base is recorded. The 3′ terminators and fluorophores from each incorporated base are removed and the incorporation, detection and identification steps are repeated. Sequencing according to this technology is described in U.S. Pat. Nos. 7,960,120; 7,835,871; 7,232,656; 7,598,035; 6,911,345; 6,833,246; 6,828,100; 6,306,597; 6,210,891; U.S. Pub. 2011/0009278; U.S. Pub. 2007/0114362; U.S. Pub. 2006/0292611; and U.S. Pub. 2006/0024681, each of which are incorporated by reference in their entirety.

Sequencing methods (e.g., NGS) generate a large number of reads either from one end of nucleic acid fragments (“single-end reads”) or from both ends of nucleic acid fragments (e.g., paired-end reads). As used herein, “reads” (e.g., “a read,” “a sequence read”) are short nucleotide sequences produced by any sequencing process described herein or known in the art. Sequencing reads, and their associated quality scores, are stored in files known as FASTQ files or FASTA files. The reads generated from any of the above-mentioned sequencing methods are aligned to a reference genome to indicate where in the genome a particular DNA fragment came from. During alignment, variations between the sample and the reference genome may be identified. The process of comparing sequence data to a reference is referred to as variant calling.

Variants refer to naturally occurring or acquired alterations to a DNA sequence that differ from the reference sequence. Variants can be classified as benign, likely benign, variant of unknown significance, likely pathogenic, or pathogenic. Both germline variants, which are present in all the body's cells, and somatic variants, which arise during the lifetime of an individual, such as in cancer, can be detected. Examples of variants include small structural variants (less than 50 base pairs) such as single nucleotide variants (SNVs), single nucleotide polymorphisms (SNPs) and small structural variants (SVs) (e.g., deletions, insertions, insertions and deletions, sometimes referred to as indels) and larger (greater than 50 base pairs) SVs such as chromosomal rearrangements (e.g., translocations and inversions). SNVs/SNPs are the result of single point mutations that can cause synonymous changes (nucleotide change does not alter the encoded amino acid), missense changes (nucleotide change does alter the encoded amino acid), or nonsense changes (resulting amino acid change converts the encoded codon to a stop codon). A sequence variation may consist of a change in, insertion of, or deletion of a single nucleotide, or of a plurality of nucleotides (e.g. 2, 3, 4, 5, 6, 7, 8, 9, 10, or more nucleotides). Where a sequence variant includes two or more nucleotide differences, the nucleotides that are different may be contiguous with one another, or discontinuous. Additional, non-limiting examples of variants further include copy number variants (CNVs), loss of heterozygosity (LOH), microsatellite instability (MSI), variable number of tandem repeats (VNTR), and retrotransposon-based insertion polymorphisms. Additional examples of types of sequence variants include those that occur within short tandem repeats (STR) and simple sequence repeats (SSR), or those occurring due to amplified fragment length polymorphisms (AFLP), differences in epigenetic marks that can be detected (e.g. methylation differences). Further, variants can occur in both coding and non-coding regions of the genome and be detected by NGS technologies. The list of detected variants and their properties (e.g., type of variant) are annotated and saved in a variant file (e.g., variant call format (VCF)).

The variant data detected using NGS can also be used to generate TMB scores. TMB, or tumor mutational burden, quantifies the total number of somatic mutations present in a tumor genome, typically reported as the number of mutations per megabase (mut/Mb) of coding DNA. The spectrum of somatic mutations contributing to TMB includes missense mutations, which alter amino acid sequences; nonsense mutations, which introduce premature stop codons; and various small insertions and deletions that can disrupt protein coding regions. The TMB score can be derived by identifying and counting non-synonymous somatic variants, including missense mutations, nonsense mutations, and small insertions or deletions, across targeted genomic regions or the entire exome using high-throughput sequencing data. TMB serves as a surrogate marker for the likelihood that a tumor will produce altered proteins, or neoantigens, which may be recognized by the immune system. Tumors with a higher TMB are generally more likely to generate immunogenic neoepitopes, making them potentially more responsive to immunotherapy such as immune checkpoint inhibitors.

TMB is calculated by sampling regions of the cancer genome, often using comprehensive genomic profiling panels or whole-exome sequencing, to estimate the overall mutation density. A high TMB score, for instance, above thresholds such as 10 mut/Mb or higher, can be associated with improved clinical response to immunotherapeutic agents. In practice, samples may be tested for high mutational burden by identifying tumors with 100, 200, 500, 1000, or more somatic mutations per genome, depending on the sequencing method and region analyzed. High mutational burden indicates a substantially greater number of somatic mutations in the tumor compared to normal tissue from the same individual. For reference, the average number of somatic mutations in non-microsatellite instable (MSS) tumors is approximately 70, whereas MSI-high tumors or tumors with DNA repair defects often exhibit much higher mutation counts. In some embodiments, the TMB score is derived directly reported from a TMB assay using the sequencing data generated by that TMB assay.

For RNA-Seq data, beyond variant detection, the aligned reads are also used to quantify gene expression levels, detect alternative splicing events, identify gene fusions, and profile transcriptome-wide changes. This quantification involves counting the number of sequencing reads that align to each gene or transcript, which provides a direct measure of gene activity in the sample. To ensure that gene expression measurements are comparable across different samples and experimental conditions, normalization methods such as TPM or FPKM are applied. These normalization techniques adjust for differences in gene length and sequencing depth, allowing for accurate comparison of expression levels both within and between samples.

In addition to gene expression quantification, RNA-Seq data can be analyzed to detect alternative splicing events. This involves identifying reads that span exon-exon junctions, which may indicate the presence of different transcript isoforms produced from a single gene. By analyzing these splice junction reads, patterns of exon inclusion or exclusion, intron retention, or usage of alternative splice sites can be characterized. RNA-Seq data also supports the identification of gene fusions, which occur when segments from two different genes are aberrantly joined together, often as a result of chromosomal rearrangements. Fusion transcripts are detected by finding reads or read pairs that map to two different genes, and their presence can serve as diagnostic or prognostic biomarkers, especially in oncology.

Beyond these targeted analyses, RNA-Seq enables comprehensive profiling of transcriptome-wide changes. Differential expression analysis can be performed to identify genes that are upregulated or downregulated between experimental groups, assess global shifts in gene expression patterns, or characterize gene signatures associated with specific biological states, such as immune activation or response to therapy. Pathway analysis and gene set enrichment analysis can be conducted to interpret the biological significance of these changes and to uncover underlying regulatory networks. Furthermore, RNA-Seq data can be integrated with other omics data, such as genomic alterations or protein expression, to provide a multi-dimensional view of the molecular landscape of a sample.

Integration of DNA and RNA sequencing results within the same workflow allows for comprehensive genomic and transcriptomic profiling, supporting multi-omics analysis for precision oncology and biomarker discovery. Multi-omics data obtained from CGIP may comprise genomic alteration data for a first set of genes and expression data for a second set of immune genes, resulting in an integrated molecular profile for the subject. Genomic alteration data may include SNVs, indels, CNVs, gene fusions, splice variants, and TMB. These alterations are detected and annotated through variant calling and structural variant analysis pipelines. The expression data can be generated by RNA-Seq or equivalent gene expression profiling assays, quantifying transcripts for a set of immune-related genes and providing information on the activity of immune pathways and tumor-immune interactions.

Optionally, the multi-omics data for the subject may further comprise IHC data, cell proliferation data, tumor inflammation data, and/or cancer testis antigen (CTA) burden data. The IHC data may include results from immunohistochemistry assays that assess the expression of specific proteins within tissue samples (e.g., obtained using the imaging system 430). For example, PD-L1 IHC data provides a quantitative or qualitative measure of PD-L1 protein expression on tumor cells or tumor-infiltrating immune cells, which can be used to guide the selection of immune checkpoint inhibitor therapy. IHC data may also capture the expression of other clinically relevant markers, such as mismatch repair proteins, HER2, or hormone receptors. The cell proliferation data can include the Ki-67 proliferation index, which is determined by staining tissue sections with antibodies against the Ki-67 nuclear protein and calculating the percentage of cells that are Ki-67 positive. The Ki-67 index can be used as a biomarker for estimating the growth fraction of a tumor and is associated with prognosis and treatment response in many cancer types. Tumor inflammation data may consist of gene expression signatures associated with immune activation or suppression within the tumor microenvironment. This can include quantification of transcripts for immune cell markers (such as CD8, CD4, or FOXP3), cytokines, chemokines, and interferon response genes. Composite inflammation signatures, such as the interferon-gamma signature or T-cell inflamed gene expression profile, can be derived to characterize the extent and nature of the immune response in the tumor. The CTA burden data may include the expression levels of one or more cancer testis antigens, which are a class of tumor-associated proteins typically restricted to germ cells in normal adult tissues but aberrantly expressed in a wide range of cancers. Measurement of CTA expression, using RNA-Seq or IHC, can support the identification of tumors that may be susceptible to CTA-targeted immunotherapies, such as cancer vaccines or adoptive T-cell therapies. The assessment of CTA burden can also contribute to tumor classification and prognostication.

By integrating SNV, indel, CNV, gene fusion, splice variant, TMB, immune gene expression, IHC, Ki-67, tumor inflammation signatures, and/or CTA expression data, the resulting multi-omics dataset enables comprehensive analysis of the genomic, transcriptomic, and phenotypic landscape of the sample. This integrated approach supports advanced analyses such as ML-based genomic instability prediction, immunophenotyping, and biomarker discovery for precision oncology. The framework allows flexible inclusion of specific data types depending on clinical or research requirements.

### 3. Storing Data

Raw or processed sequencing files from genomic profiling or immune profiling may be stored in a storage device 440, such as a server, a database, or a data repository. These files can include formats such as FASTQ, BAM, VCF, or normalized gene expression matrices, depending on the stage of analysis. Storage can be local, such as on a laboratory server or internal database, or remote, including secure cloud-based servers that support large-scale data storage and sharing. During analysis, one or more files may further be transmitted to another system (e.g., a machine learning training and deployment system, as described in further detail herein). Secure data transfer protocols and access controls can be used to protect the confidentiality and integrity of the sequencing data throughout these processes.

In some embodiments, particularly when training a machine learning model, it is not necessary to collect biological samples or perform laboratory-based sample processing as described above (e.g., steps at block 405, 410, 435, 445, 450, or 455). Instead, the training data may consist of multi-omics data (gene expression data, genomic alteration data, and genomic instability data, all corresponding to training samples) that are obtained directly from public, private, or commercial databases. Publicly available resources, such as the Cancer Genome Atlas (TCGA) and Gene Expression Omnibus (GEO), offer access to large and well-curated multi-omics datasets. These databases contain transcriptomic profiles, mutation and variant information, genomic instability metrics, and a variety of clinical annotations for many different cancer types and research cohorts. In addition to public repositories, institutional databases and commercial providers may also supply suitable training data. Once acquired, these data can be securely stored in a storage device, such as storage device 440, to support further processing, harmonization, and analysis. The storage device 440 functions as a centralized repository, making it possible to efficiently manage, integrate, and retrieve these diverse data types for downstream computational tasks. Such tasks may include feature extraction, machine learning model training, and model validation. Using these external sources may help improve the efficiency of developing and optimizing predictive models, since there is no need for direct sample collection or laboratory profiling when high-quality, curated training data are already available from established databases.

### VI. TRAINING AND USING A MACHINE LEARNING MODEL FOR AI-BASED GENOMIC INSTABILITY PREDICTION

FIG. 7 shows a block diagram of a machine learning pipeline 700 comprising several subsystems that work together to train, validate, and implement one or more machine learning models to predict genomic instability status in accordance with various embodiments. As discussed previously, conventional genomic instability detection methods, such as standard sequencing or immunohistochemistry, can fail to yield results or may not capture the complex, multi-dimensional patterns present in multi-omics data. Machine learning models are uniquely suited to learn from large and diverse datasets that integrate genomic alteration data, gene expression profiles, and immune-related features. By leveraging these models, genomic instability status such as HRD and MSI can be predicted even when traditional tests are inconclusive or limited by sample quality and technical constraints.

The machine learning pipeline 700 comprises a data subsystem 705 for collecting, generating, preprocessing, and labeling of training and validation datasets 710, training and validation subsystem 715 that facilitates the training and validation of one or more machine learning algorithms 720, and inference subsystem 725 for deploying and implementing one or more trained machine learning models 730 independently or in combination with one or more other systems or services 735 for downstream processes.

As used herein, machine learning algorithms (also described herein as simply algorithm or algorithms) are procedures that are run on datasets (e.g., training and validation datasets) and perform pattern recognition on datasets, learn from the datasets, and/or are fit on the datasets. Examples of machine learning algorithms include linear and logistic regression, decision trees, artificial neural networks, k-means, k-nearest neighbor, Classification and Regression Trees (CART), Gradient Boosting Machine (GBM), K-Nearest Neighbors (K-NN), Linear Discriminant Analysis (LDA), Logistic Regression, Multi-Layer Perceptron (MLP), Naive Bayes, Random Forest, Support Vector Machine with Radial Basis Function Kernel (SVM Radial), Boruta algorithm, and Extreme Gradient Boosting (XGBoost). Machine learning models (also described herein as simply model or models) are the output of the machine learning algorithms and are comprised of model data and a prediction algorithm. In other words, the machine learning model is the program that is saved after running a machine learning algorithm on training data and represents the rules, numbers, and any other algorithm-specific data structures required to make inferences. For example, a linear regression algorithm may result in a model comprised of a vector of coefficients with specific values, a decision tree algorithm may result in a model comprised of a tree of if-then statements with specific values, or neural network, backpropagation, and gradient descent algorithms together result in a model comprised of a graph structure with vectors or matrices of weights with specific values.

### 1. Data Subsystem

Data subsystem 705 is used to collect, generate, preprocess, and label data to be used to train and validate one or more machine learning algorithms 720. The data subsystem 705 can be specifically designed to handle multi-omics data, which includes both genomic alteration data (such as SNVs, indels, CNVs, gene fusions, splice variants, and TMB) and gene expression data (such as transcriptomic profiles for immune genes or tumor inflammation signatures), as well as optional data types like IHC data (e.g., PD-L1 IHC), cell proliferation data (e.g., Ki-67 proliferation index), tumor inflammation data (e.g., gene expression signatures associated with inflammation), and CTA burden data (e.g., CTA expression levels). The data collection can include exploring various data sources such as public datasets, private data collections, or real-time data streams, depending on a project's needs. In some instances, a data source is a public or online repository of information or examples pertinent to a general or target domain space. Many domains have publicly available datasets provided by governments, universities, or organizations. For example, many government and private entities offer datasets on healthcare, environmental data, and more through various portals. For proprietary needs, data might be available through partnerships or purchases from private companies that specialize in data aggregation.

For example, data sources may include comprehensive cancer databases such as TCGA or GEO, as well as commercial panels that provide integrated DNA and RNA sequencing data covering clinically relevant genes and immune markers. In certain instances, a data source can be the storage device 440 (described with respect to FIG. 4) that stores digital images and raw and/or processed sequencing files from NGS or RNA sequencing assays. In other instances, a data source can be a public or private database that includes DNA and RNA sequencing data covering cancer-relevant genes. Once a data source is identified, data subsystem 705 can be used to collect data through appropriate methods such as downloading from online repositories, web scraping, using APIs for real-time data, creating datasets through surveys and experiments, or by deploying sensors in the environment. The inclusion of multi-omics data in the data subsystem 705 enables the machine learning pipeline to integrate diverse molecular features, supporting more accurate and comprehensive model training for genomic instability prediction.

Data synthesis and/or data augmentation techniques may be implemented using data subsystem 705 to generate data to be used for the training and validation datasets 710 (e.g., when data is insufficient from public datasets and/or private data collections). Data synthesis involves creating entirely new data points, which can be especially useful when actual patient data are scarce, highly sensitive, or prohibitively expensive to collect. For example, Generative Adversarial Networks (GANs) can be trained on existing multi-omics datasets—such as gene expression matrices or genomic alteration profiles—to generate synthetic samples that mimic the distribution of real patient data. Variational Autoencoders (VAEs) can also be used to simulate new transcriptomic profiles or variant sets that reflect the complexity and variability of actual biological samples. These synthesized data points help increase the diversity of the training set, provided that they remain realistic and compliant with applicable privacy or copyright regulations.

Data augmentation refers to techniques that artificially expand the dataset by creating modified versions of existing data points. For instance, in the case of pathology images, augmentation may involve rotating, flipping, scaling, or changing the color balance of IHC images to simulate variability in slide preparation and imaging conditions. For RNA-Seq or other tabular omics data, augmentation can include introducing small amounts of noise to gene expression values, randomly shuffling feature order, or simulating batch effects. In text-based clinical records, augmentation may use synonym replacement or sentence reordering. The primary goal of these techniques is to enhance the robustness of the machine learning model, making it more resilient to variability in real-world data and improving its ability to generalize to unseen samples.

In some embodiments, data imputation can be implemented using the data subsystem 705 to address the challenge of missing values in training or validation datasets. Missing data can arise for many reasons, including incomplete sequencing coverage, technical assay failures, or variability in sample quality. To ensure that machine learning models are trained on complete and consistent datasets, imputation methods are used to estimate and fill in these missing values. For example, in genomic profiling, missing gene expression values may be imputed using statistical techniques such as mean or median substitution, k-nearest neighbors, or more advanced methods like MICE. For categorical features, such as mutation presence or absence, imputation can involve inferring likely values based on patterns observed in the remaining data. In the context of high-dimensional multi-omics data, machine learning-based imputation approaches, such as predictive mean matching, matrix factorization, or model-based regression, can leverage correlations among features to produce more accurate estimates. By applying data imputation, the analysis pipeline maintains the integrity of the dataset, prevents bias from case-wise deletion, and allows robust training of models that can generalize well to real-world, heterogeneous clinical samples.

Preprocessing may be implemented using data subsystem 705 in the data collection process, serving as a bridge between raw data acquisition and effective model training. The primary objective of preprocessing is to transform raw data into a format that is more suitable and efficient for analysis, ensuring that the data fed into machine learning algorithms is clean, consistent, and relevant. This step can be useful because raw data often comes with a variety of issues such as missing values, noise, irrelevant information, and inconsistencies that can significantly hinder the performance of a model. By standardizing and cleaning the data beforehand, preprocessing helps in enhancing the accuracy and efficiency of the subsequent analysis, making the data more representative of the underlying problem the model aims to solve.

Several example techniques implemented in preprocessing include data cleaning, normalization, feature extraction, and dimensionality reduction. Data cleaning may involve removing duplicates, filling in missing values, or filtering out outliers to improve data quality. Normalization involves scaling numeric values to a common scale without distorting differences in the ranges of values, which helps prevent biases in the model due to the inherent scale of features.

Feature extraction and feature selection may be conducted both per data category and across the entire dataset. For example, features can be extracted separately from genomic data, such as SNVs, indels, or CNVs, and from immune data, such as gene expression signatures or immune cell infiltration markers. These extracted features may then be integrated to form a comprehensive multi-omics dataset. Additionally, feature extraction may target combined patterns across all data types, enabling the identification of cross-modality interactions that are relevant for predicting genomic instability.

In some embodiments, feature selection is performed separately for each CGIP data type, such as gene expression and SNV data, to identify candidate features most relevant (e.g., top 10%) for predicting genomic instability. For example, a feature-selection model, such as a Boruta algorithm or a random forest model, may be used to compare the importance of original features to that of randomly permuted shadow features. Features confirmed by the algorithm as statistically significant predictors are selected for model training. The final feature set may include genomic, transcriptomic, and optional immunophenotypic variables, such as markers from IHC data, as well as calculated parameters like TMB. The selected features are further validated for stability and relevance by applying cross-validation within the training data, ensuring that the chosen features generalize well to unseen samples. Feature selection may also be performed on the entire multi-omics dataset to identify globally important predictors.

Dimensionality reduction techniques like Principal Component Analysis (PCA) or Autoencoders may be used to reduce the number of random variables under consideration, by obtaining a set of principal variables. These techniques not only help in reducing the computational load on the model but also in mitigating issues like overfitting by simplifying the data without losing critical information.

In the instance that machine learning pipeline 700 is used for supervised or semi-supervised learning of machine learning models, labeling techniques can be implemented as part of the data collection. The quality and accuracy of data labeling directly influence the model's performance, as labels serve as the definitive guide that the model uses to learn the relationships between the input features and the desired output. Particularly in complex domains such as image recognition, natural language processing, or medical diagnosis, precise and consistent labeling is important because it provides the ground truth or target outcomes against which the model's predictions are compared and adjusted during training. Effective labeling ensures that the model is trained on correct and clear examples, thus enhancing its ability to generalize from the training data to real-world scenarios. In various embodiments, labeling specifically involves annotating each training sample with its known genomic instability status. For example, a sample might be labeled as “HRD-high” if it shows homologous recombination deficiency, “HRD-low” if homologous recombination is intact, “MSI-high” if it exhibits microsatellite instability, or “MSS” if it is microsatellite stable. These labels are assigned based on corresponding labeling data saved in the databases or gold-standard results from prior conventional assays, such as PCR-based fragment analysis, immunohistochemistry, or validated next generation sequencing workflows. For multi-omics datasets, each data point may also be linked to clinical metadata, such as cancer type, treatment history, or outcome, further refining the labeling process.

Labeling techniques can vary significantly depending on the type of data and the specific requirements of the project. Manual labeling, where human annotators label the data, is one method that can be used. This approach may be useful when a detailed understanding and judgment are required, such as in labeling medical images or categorizing text data where context and subtlety are important. However, manual labeling can be time-consuming and prone to inconsistency, especially with a large number of annotators. To mitigate this, semi-automated labeling tools may be used as part of data subsystem 705 to pre-label data using algorithms, which human annotators may then review and correct as needed. For example, semi-automated systems might use rule-based algorithms to pre-assign likely MSI or HRD status based on thresholds in mutational burden or gene expression signatures, with human reviewers confirming or correcting these assignments. Another approach is active learning, a technique where the model being developed is used to label new data iteratively. The model suggests labels for new data points, and human annotators may review and adjust certain predictions such as the most uncertain predictions. This technique optimizes the labeling effort by focusing human resources on a subset of the data, e.g., the most ambiguous cases, improving efficiency and label quality through continuous refinement.

Once the data is collected, preprocessed, and labeled, the data subsystem splits the complete dataset into training, validation, and testing sets (e.g., training and validation datasets 710). The split may be performed using a random strategy, stratified sampling, or k-fold cross-validation, ensuring that each subset maintains the distribution of genomic instability statuses and relevant clinical variables. For example, in stratified sampling, the proportion of HRD-high, HRD-low, MSI-high, and MSS cases is preserved across all subsets, preventing class imbalance and supporting fair model evaluation. The data collected is typically split into at least three subsets: training, validation, and testing. The training set is used to fit the model, where the machine learning model learns to make inferences based on the training data. The validation set, on the other hand, is utilized to tune hyperparameters and prevent overfitting by providing a sandbox for model selection. Finally, the test set serves as a new and unseen dataset for the model, used to simulate real-world application and evaluate the final model's performance. The process of splitting ensures that the model can perform well not just on the data it was trained on, but also on new, unseen data, thereby validating and testing its ability to generalize.

Various techniques can be employed to split the data effectively, with each method aiming to maintain a good representation of the overall dataset in each subset. A simple random split (e.g., a 70/20/10%, 80/10/10%, or 60/25/15%) is the most straightforward approach, where examples from the data are randomly assigned to each of the three sets. However, more sophisticated methods may be necessary to preserve the underlying distribution of data. For instance, stratified sampling may be used to ensure that each split reflects the overall distribution of a specific variable, particularly useful in cases where certain categories or outcomes are underrepresented. Another technique, k-fold cross-validation, involves rotating the validation set across different subsets of the data, maximizing the use of available data for training while still holding out portions for validation. These methods help in achieving more robust and reliable model evaluation and are useful in the development of predictive models that perform consistently across varied datasets.

Data subsystem 705 is also used to set and implement hyperparameters 740 to be optimized by the training and validation subsystem 715. Hyperparameters are configuration settings that govern the overall behavior and structure of machine learning models before the training process begins. Unlike model parameters 745, which are automatically learned and updated by the model during training, hyperparameters can be defined in advance and can significantly influence the learning process and final model performance.

For example, in the context of neural networks, hyperparameters may include the learning rate, which determines how much the model updates its weights with each iteration; the number of layers in the network; the number of neurons or nodes in each layer; the choice of activation functions; the width of convolutional kernels; the number of kernels for a given layer; the number of graph connections established during a lookback period; and the maximum depth of a decision tree in a random forest. In the context of a random forest model, hyperparameters include the number of trees in the forest, the maximum depth allowed for each tree, the minimum number of samples required to split a node, the minimum number of samples required to be at a leaf node, and the number of features considered when looking for the best split at each node. Adjusting these hyperparameters directly impacts the model's predictive accuracy, robustness, and computational efficiency. Other examples include the batch size used during training, the number of training epochs, the regularization strength to prevent overfitting, or the minimum number of samples required to split an internal node in a tree-based model.

These hyperparameter settings can determine how quickly the model learns, how well it generalizes from the training data to unseen samples, and the overall complexity and interpretability of the resulting model. Selecting appropriate hyperparameter values is critical because poor choices can lead to models that either underfit or overfit the data. Underfitting occurs when a model is too simple to capture the underlying patterns in the data, resulting in poor predictive performance. Overfitting, on the other hand, happens when a model is too complex and starts to learn random noise or artifacts in the training data as if they were meaningful signals, which reduces its ability to generalize to new data. By carefully setting and optimizing hyperparameters through the data subsystem 705 and the training and validation subsystem 715, the machine learning pipeline supports the development of robust and high-performing models for genomic instability prediction.

### 2. Training, Validation, and Testing

The training and validation subsystem 715 is comprised of a combination of specialized hardware and software to efficiently handle the computational demands required for training, validating, and testing a machine learning model. On the hardware side, high-performance GPUs (Graphics Processing Units) may be used for their ability to perform parallel processing, drastically speeding up the training of complex models, especially deep learning networks. CPUs (Central Processing Units), while generally slower for this task, may also be used for less complex model training or when parallel processing is less critical. TPUs (Tensor Processing Units), designed specifically for tensor calculations, provide another level of optimization for machine learning tasks. On the software side, a variety of frameworks and libraries are utilized, including TensorFlow, PyTorch, Keras, and scikit-learn. These tools offer comprehensive libraries and functions that facilitate the design, training, validation, and testing of a wide range of machine learning models across different computing platforms, whether local machines, cloud-based systems, or hybrid setups, enabling developers to focus more on model architecture and less on underlying computational details.

Training is the initial phase of developing machine learning models 730 where the model learns to make predictions or decisions based on data training data provided from the training and validation datasets 710. During this phase, the model iteratively adjusts its internal model parameters 745 to minimize the difference between its predictions and the actual outcomes in the training data. This process, known as fitting, is fundamental because it directly influences the accuracy and effectiveness of the model. The training phase is driven by three primary components: the model architecture (which defines the structure of the algorithm(s) 720), the training data (which provides the examples from which to learn), and the learning algorithm (which dictates how the model adjusts its model parameters). The goal is for the model to capture the underlying patterns of the data without memorizing specific examples, thus enabling it to perform well on new, unseen data.

The model architecture refers to the specific arrangement and structure of the components and layers that make up a machine learning model, as well as the algorithms and feature selection strategies employed. Tree-based model architectures, including random forest models and recursive partitioning and regression tree (rpart) models, are effectiveness in handling multi-omics data for genomic instability prediction. The architecture determines how input data-including genomic alteration data, gene expression features, and optional immunophenotypic variables—are processed and transformed through sequential or branched computational steps to yield the final output.

For tree-based models, the architecture involves constructing a series of hierarchical decision nodes, where each node splits the data according to the value of a specific feature. In a random forest, multiple decision trees are built in parallel, each trained on a bootstrapped subset of the data and using a randomly selected subset of features at each split. This ensemble strategy reduces variance, improves generalization, and provides robust predictions even when individual features are noisy or missing. The rpart model is a single decision tree model that partitions the data based on feature values to maximize homogeneity of the outcome variable within each terminal node. Both random forest and rpart models are well-suited to high-dimensional, heterogeneous, and partially missing data, which are common characteristics of multi-omics datasets.

The architecture directly influences the model's ability to learn from the data effectively and efficiently. Random forest and rpart models can automatically capture complex, non-linear relationships between genomic instability status and multi-omics features without requiring extensive data transformation or manual variable engineering. These models are interpretable, as the decision paths can be visualized and analyzed for feature importance, which is critical for clinical transparency and regulatory compliance. Moreover, tree-based architectures are memory-efficient, since they require only the storage of split rules and terminal node values, and they allow for fast inference by quickly traversing the tree structure. The use of random forest and rpart models in this invention provides several technical improvements and benefits over conventional approaches. These include enhanced handling of missing or noisy data, built-in feature selection through assessment of variable importance, reduced risk of overfitting due to ensembling or pruning strategies, and scalability to large, multi-omics datasets. These models also offer rapid training and inference times, which is essential for clinical applications where real-time or near-real-time predictions may be required. By selecting the optimal algorithmic architecture based on the specific characteristics of the input data and the clinical prediction task, the system achieves high sensitivity, specificity, and robustness in predicting genomic instability status, as validated through rigorous cross-validation and performance benchmarking.

The model architecture can encompass a wide range of algorithms 720, each suitable for different tasks and data types, the architecture can be designed to support not only random forest and rpart models but also other supervised and unsupervised machine learning algorithms, including but not limited to Classification and Regression Trees (CART), Gradient Boosting Machine (GBM), K-Nearest Neighbors (K-NN), Linear Discriminant Analysis (LDA), Logistic Regression, Multi-Layer Perceptron (MLP), Naive Bayes, Support Vector Machine with Radial Basis Function Kernel (SVM Radial), Extreme Gradient Boosting (XGBoost), Boruta algorithm, and deep learning models such as convolutional neural networks (CNNs) and recurrent neural networks (RNNs). These algorithms can be implemented using a variety of machine learning libraries and frameworks, such as scikit-learn for tree-based models and ensemble methods, or TensorFlow and PyTorch for deep learning architectures.

The learning algorithm is the overall method or procedure used to adjust the model parameters 745 to fit the data. It dictates how the model learns from the data provided during training, including the rules and steps followed to process input data and refine the model's internal structure. In this invention, learning algorithms are specifically chosen and implemented to optimize the performance of machine learning models for genomic instability prediction using multi-omics data. For example, tree-based models such as random forests and rpart models use recursive partitioning and splitting criteria (such as information gain or Gini impurity) to select the most informative features and build decision trees that classify samples based on their molecular and phenotypic characteristics. Other learning algorithms, such as those for neural networks, use gradient-based optimization and backpropagation to minimize classification or regression loss functions.

Various techniques may be employed by training and validation subsystem 715 to train machine learning models 730 using the learning algorithm, depending on the type of model and the specific task. For supervised learning models, where the training data includes both inputs and expected outputs (e.g., ground truth labels), gradient descent is a possible method. This technique iteratively adjusts the model parameters 745 to minimize or maximize an objective function (e.g., a loss function, a cost function, a contrastive loss function, etc.). The objective function is a method to measure how well the model's predictions match the actual labels or outcomes in the training data. It quantifies the error between predicted values and true values and presents this error as a single real number. The goal of training is to minimize this error, indicating that the model's predictions are, on average, close to the true data. Common examples of loss functions include mean squared error for regression tasks and cross-entropy loss for classification tasks. For decision tree and random forest models, the algorithm repeatedly splits the training data based on attribute value tests, recursively partitioning the dataset to maximize the purity of the resulting subsets. In some embodiments, training of models can be performed using the train function in the caret R package, with 10-fold cross-validation employed to ensure robust model evaluation. During each fold, the model is trained on a subset of the data and validated on the held-out portion, with the process repeated ten times to assess stability and generalizability of the learned patterns.

The adjustment of the model parameters 745 is performed by the optimization function or algorithm, which refers to the specific method used to minimize (or maximize) the objective function. The optimization function is the engine behind the learning algorithm, guiding how the model parameters 745 are adjusted during training. It determines the strategy to use when searching for the best weights that minimize (or maximize) the objective function. For example, in neural networks, stochastic gradient descent or advanced variants like Adam or RMSprop are used to optimize the network's weights. In tree-based models, recursive partitioning and pruning are used to select optimal splits and prevent overfitting. Once trained, models are evaluated using comprehensive validation metrics. In the described workflow, the trained models are used to predict MSI status in independent test sets, using the predict function in caret. Model predictions are then compared to ground truth MSI status, with performance assessed using the confusionMatrix function in caret. Other common evaluation metrics include accuracy, specificity, positive predictive value (PPV), negative predictive value (NPV), recall or sensitivity, F1 score, and balanced accuracy.

Further, model evaluation is extended to additional unseen cohorts to assess generalizability. Receiver operating characteristic (ROC) and precision-recall (PR) curves are generated, and area under the curve (AUC) is calculated for each cohort using the top performing model. AUC-ROC and AUC-PR metrics can provide a comprehensive assessment of model discrimination and predictive power. The best performing model across all cohorts is then applied to cases that previously failed conventional microsatellite testing to demonstrate the feasibility and utility of using the machine learning pipeline for real-world genomic instability determination when standard assays are inconclusive or unsuccessful.

In unsupervised learning, where training data does not include labels, different techniques are used. Clustering is one method where data is grouped into clusters that maximize the similarities of data within the same cluster and maximize the differences with data in other clusters. The K-Means algorithm, for example, assigns each data point to the nearest cluster by minimizing the sum of distances between data points and their respective cluster centroids. Another technique, Principal Component Analysis (PCA), involves reducing the dimensionality of data by transforming it into a new set of variables, the principal components, which are uncorrelated and ordered so that the first few retain most of the variation present in all of the original variables. These techniques help uncover hidden structures or patterns in the data, which can be essential for feature reduction, anomaly detection, or preparing data for further supervised learning tasks.

Validating is another phase of developing machine learning models 730 where the model is checked for deficiencies in performance and the hyperparameters 740 are optimized based on validation data provided from the training and validation datasets 710. The validation data helps to evaluate the model's performance, such as accuracy, precision, recall, or F1-score, to gauge how well the model is likely to perform in real-world scenarios. Hyperparameter optimization, on the other hand, involves adjusting the settings that govern the model's learning process (e.g., learning rate, number of layers, size of the layers in neural networks) to find the combination that yields the best performance on the validation data. One optimization technique is grid search, where a set of predefined hyperparameter values are systematically evaluated. The model is trained with each combination of these values, and the combination that produces the best performance on the validation set is chosen. Although thorough, grid search can be computationally expensive and impractical when the hyperparameter space is large. A more efficient alternative optimization technique is random search, which samples hyperparameter combinations from a defined distribution randomly. This approach can in some instances find a good combination of hyperparameter values faster than grid search. Advanced methods like Bayesian optimization, genetic algorithms, and gradient-based optimization may also be used to find optimal hyperparameters more effectively. These techniques model the hyperparameter space and use statistical methods to intelligently explore the space, seeking hyperparameters that yield improvements in model performance.

Once a machine learning model has been trained and validated, it undergoes a final evaluation using test data provided from the training and validation datasets 710, which is a separate subset of the data that has not been used during the training or validation phases. This step is crucial as it provides an unbiased assessment of the model's performance in simulating real-world operation. The test dataset serves as new, unseen data for the model, mimicking how the model would perform when deployed in actual use. During testing, the model's predictions are compared against the true values in the test dataset using various performance metrics such as accuracy, precision, recall, and mean squared error, depending on the nature of the problem (classification or regression). This process helps to verify the generalizability of the model-its ability to perform well across different data samples and environments-highlighting potential issues like overfitting or underfitting and ensuring that the model is robust and reliable for practical applications. The machine learning models 730 are fully validated and tested once the output predictions have been deemed acceptable by user defined acceptance parameters. Acceptance parameters may be determined using correlation techniques such as Bland-Altman method and the Spearman's rank correlation coefficients and calculating performance metrics such as the error, accuracy, precision, recall, receiver operating characteristic curve (ROC), etc.

### 3. Inference Phase for Machine Learning Models

The inference subsystem 725 is comprised of various components for deploying the machine learning models 730 in a production environment. Deploying the machine learning models 730 includes moving the models from a development environment (e.g., the training and validation subsystem 715, where it has been trained, validated, and tested), into a production environment where it can make inferences on real-world data (e.g., input data 750). This step typically starts with the model being saved after training, including its parameters and configuration such as final architecture and hyperparameters. It is then converted, if necessary, into a format that is suitable for deployment, depending on the deployment environment. For instance, a model trained in a scientific computing environment such as Python might be converted into a Java-friendly format for integration into a larger enterprise application. Deployment can be conducted on various platforms, including on-premises servers, cloud environments like AWS, Azure, Google.

In various embodiments, the deployed models are configured to predict genomic instability status, such as homologous recombination deficiency (HRD) or microsatellite instability (MSI), by leveraging integrated multi-omics data that include genomic alteration, gene expression, and immunophenotypic features. Deployment of these models enables automated, accurate, and rapid determination of genomic instability status in real-world clinical and laboratory contexts. This approach addresses the limitations of traditional single-modality and rule-based techniques by allowing the system to process complex, high-dimensional data in a scalable and reproducible way.

Once deployed, the model is ready to receive input data 750, which may include multi-omics features generated from patient samples, in order to produce output predictions (inferences 755) of genomic instability status. The technical benefit of this deployment is that clinicians, laboratory staff, or automated systems can submit genomic and immune data for a patient and receive a rapid prediction of HRD, MSI, or other instability status, along with supporting metrics such as confidence scores or key contributing features. This also enables the use of the disclosed techniques as a component of a larger clinical decision support system, a research workflow, or a diagnostic service. Integration can be accomplished through user-friendly interfaces or through APIs that connect the deployed model with other applications and databases. The application layer manages data formatting, quality control, and communication, ensuring that input data from a storage device 440 described with respect to FIG. 4 or multi-omics databases is properly prepared for model inference. The model then processes the data, applies the trained decision logic, and returns actionable predictions to support clinical or research decisions. This deployment directly contributes to improved diagnostic accuracy, therapy selection, or research insights, particularly in cases where conventional testing methods are inconclusive or incomplete.

To ensure ongoing performance, the deployed model is continuously monitored in the production environment. This monitoring includes tracking prediction accuracy, response times, and user feedback, as well as evaluating operational metrics to detect any signs of model drift or declining reliability. Technical improvements provided by the trained machine learning models, such as the use of random forest and rpart architectures, include enhanced robustness to missing or noisy data, high interpretability of feature importance, and computational efficiency suitable for high-throughput laboratory and clinical workflows. The system is designed to support retraining or updating of the model as new data are collected or as clinical standards evolve. This ensures that predictive accuracy and clinical relevance are maintained over time. Ongoing evaluation and maintenance of the deployed model preserve the trustworthiness and real-world utility of the solution in dynamic clinical and research settings. The deployment of these advanced machine learning models provides a scalable, automated, and highly accurate solution for genomic instability testing, directly supporting precision medicine and timely patient care.

### VII. GRADIENT BOOSTING DECISION TREE MACHINE LEARNING MODEL

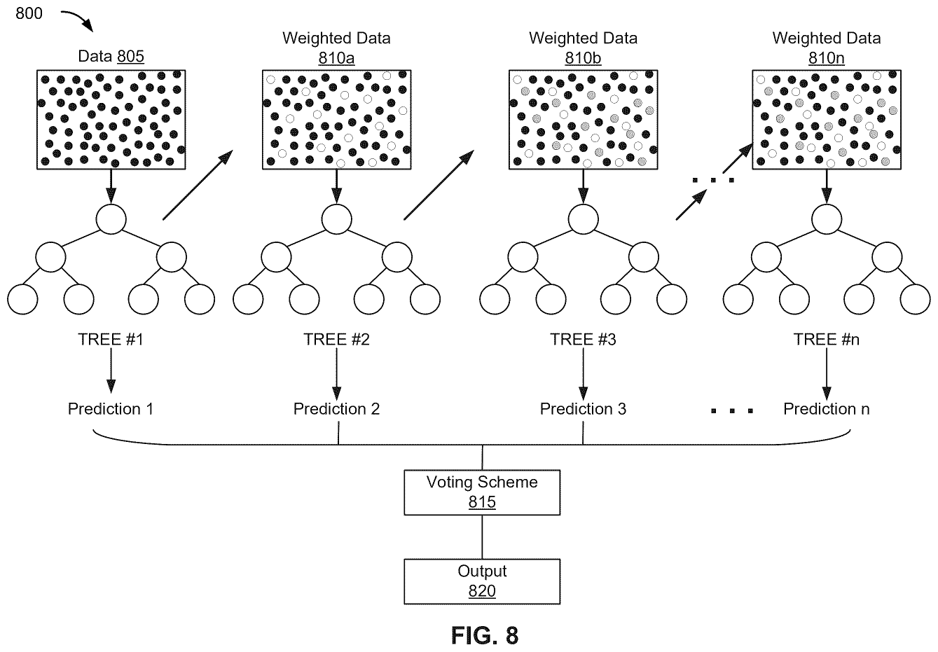

FIG. 8 shows an exemplary illustration of a gradient boosting decision tree machine learning model 800 in accordance with various embodiments. The gradient boosting decision tree machine learning model 800 is an example of a tree-based architecture that is particularly well suited for genomic instability prediction, such as MSI or HRD status. Gradient boosting models are highlighted here because they have distinct technical advantages when analyzing high-dimensional, multi-omics data that may be noisy or partially missing. Unlike conventional rule-based or single-model approaches, gradient boosting assembles a strong and accurate predictive model by iteratively combining multiple weak learners, here implemented as individual decision trees. Other tree-based models, such as RF and rpart, may also be suitable for genomic instability prediction and may be used in alternative embodiments.

The goal of gradient boosting decision tree models is to combine many weak individual learners (e.g., individual decision trees) to generate a strong learner. This is achieved because the trees are connected in a series and thus each tree minimizes the error from the previous tree, making the model 800 highly accurate. In other words, the gradient boosting decision tree machine learning model 800 builds an initial model (e.g., tree #1) using dataset 805 (e.g., CGIP dataset). The initial model makes predictions for all samples and calculates the error by comparing predicted and actual values of the genomic instability status.

For those samples that are misclassified or poorly predicted, the model assigns greater weight, generating a new weighted dataset 810a. Then the model 800 creates another tree (e.g., tree #2) that attempts to fix the errors from the last tree. A second round of predictions are made using the entire weighted dataset 810b. This process repeats generating sequential ‘n’ sequential trees, with each new tree aiming to correct the errors generated by the previous tree. Each decision tree in the gradient boosting model may vary in its depth or structure, allowing the model to adapt to the complexity of the data and to specific predictive challenges.

During the construction of each decision tree, feature selection is inherently integrated. Rather than evaluating all possible features for every split, the algorithm may consider a random subset of features at each node, introducing diversity and helping the model capture complex relationships. For example, out of hundreds of features, including gene expression ranks, mutation presence or absence, and immune scores, each tree may use a unique combination of 10 to 1000 features for its splits. This targeted selection enhances model interpretability and accuracy.

Each decision tree is a decision support tool that uses a binary tree graph to make decisions and/or predict their possible consequences. In constructing a gradient boosting decision tree model 800, each decision tree is constructed independently based on a random subset of the training data (e.g., features identified during feature selection). For example, when constructing each decision tree, instead of considering all the features in the training data for each split, a random subset of features (Ni features) is generally selected, which helps introduce randomness and diversity among the decision trees in the forest. Across the ensemble, the trees collectively learn to distinguish between cases such as MSI-high and MSS or HRD-high and HRD-low, based on the patterns in the input features.

A significant technical advantage of the gradient boosting decision tree model is its ability to correct errors sequentially, which leads to improved predictive accuracy over single-tree approaches. The gradient boosting trees are built in sequence, allowing each new tree to focus on the specific errors of the previous trees. This makes the model especially effective for datasets where subtle interactions or nonlinearities between features are important for prediction, such as in multi-omics data.

Additionally, these models are computationally efficient and memory-saving. The structure of the model, which only stores split rules and terminal node outcomes, avoids the need for large parameter matrices. This efficiency is crucial for applications in clinical or laboratory settings with limited computing resources. The model can also handle missing values during both training and inference. If a feature needed for a split is missing, surrogate splitting is used, where another highly correlated feature is chosen to make the decision. If no suitable surrogate is available, default values such as the mean or median from the training data may be used, ensuring that reliable predictions are still produced without discarding incomplete samples.

At the end of the sequential learning process, the gradient boosting decision tree machine learning model 800 uses a voting scheme 815 to combine the outputs from all of the individual decision trees and produce a final, robust prediction 820. In the context of classification tasks, such as distinguishing between MSI-high and MSS or HRD-high and HRD-low, the voting scheme can be implemented as majority voting. In majority voting, each tree in the ensemble generates its own classification for the input sample, and the final class assigned to the sample is the one that receives the most votes across all trees. This approach increases prediction accuracy and stability, as it reduces the influence of any single tree that may have made an error due to noise or outlier features.

Alternatively, in the case of regression or probability-based outputs, the voting scheme 815 may involve calculating a weighted mean of the predictions produced by each tree. In this method, each tree contributes a probability score or a numerical value, and these values are averaged, sometimes with weights assigned based on the confidence or performance of each tree during training, to generate the final output prediction 820. This weighted aggregation allows the model to reflect the collective assessment of all trees, capturing subtle patterns that may be detected by different parts of the ensemble.

The choice of voting scheme is another technical advantage because it enables the gradient boosting decision tree machine learning model 800 to deliver highly accurate and reliable predictions. By aggregating the decisions of many weak learners, the voting scheme mitigates overfitting, smooths out individual errors, and provides a consensus result that is more robust to variation in the input data. This ensemble decision-making process also allows the model to handle missing or incomplete data more effectively, since not every tree needs every feature to make a decision. Furthermore, the voting scheme supports interpretability, as the contribution of each tree and the importance of each feature in the final prediction can be analyzed and reported, aiding in clinical validation and regulatory review.

### VIII. EXAMPLES, MATERIALS, AND METHODS

The following examples are provided by way of illustration only and not by way of limitation. Those of skill in the art will readily recognize a variety of non-critical parameters that could be changed or modified to yield essentially the same or similar results.

### a) Patient Cohorts

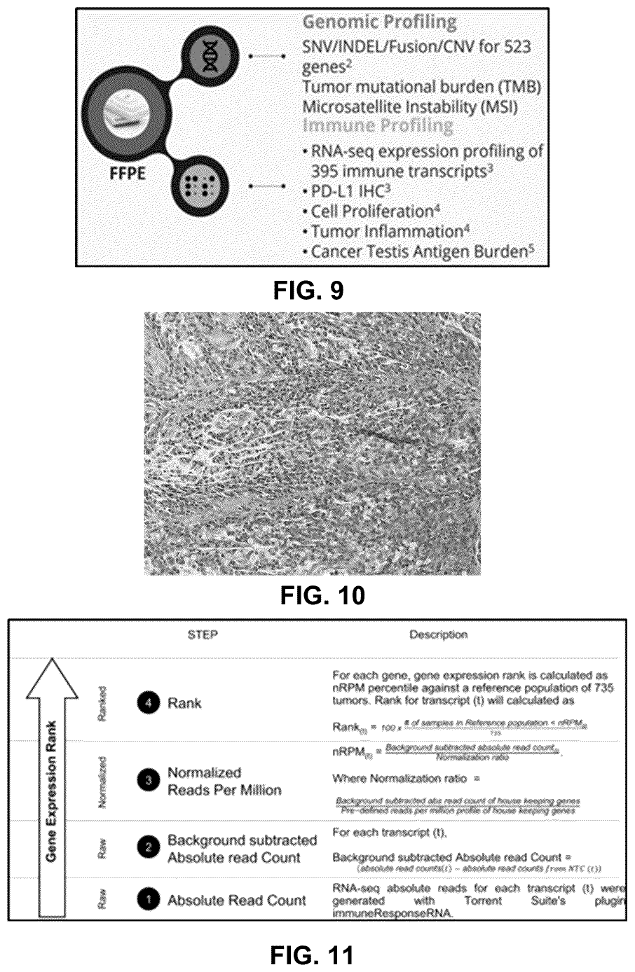

A retrospective cohort of 2,469 colorectal cancer (CRC) cases with comprehensive genomic and immune profiling (CGIP) data were analyzed using the OmniSeq® INSIGHT assay (FIG. 9, Table 1). Cases passing microsatellite testing (N=2,282, 92%) were randomly split into a training (N=1,597, 70%) and testing (N=685, 30%) dataset to use for training and validating machine learning classification models. Additionally, data for a cohort of colon adenocarcinoma (COAD) and rectum adenocarcinoma (READ) cases were extracted from The Cancer Genome Atlas (TCGA) data portal to use as an additional, independent validation dataset for trained classification models (Table 1). A cohort of uterine cancer (UC) cases tested via CGIP (N=449) were analyzed to determine the prediction capabilities of trained classification models in UC (Table 1).

TABLE 1
Patient characteristics

| | | | Mini- | Maxi- | | MSI- |
|---|---|---|---|---|---|---|
| | Sample | Median | mum | mum | Female | High |
| Cohort | Size (n) | Age (y) | Age (y) | Age (y) | (%) | (%) |
| CRC training | 1597 | 65 | 24 | ≥90 | 45% | 6% |
| CRC testing | 685 | 65 | 29 | ≥90 | 46% | 9% |
| TCGA- | 364 | 66 | 31 | ≥90 | 47% | 14% |
| COAD/ | | | | | | |
| READ | | | | | | |
| UC | 449 | 69 | 26 | ≥90 | 100% | 16% |
| CRC MSI | 187 | 65 | 26 | 87 | 46% | 8%* |
| failures | | | | | | |

*Predicted using trained CART model

Finally, data for CRC cases that failed microsatellite testing (N=187, 8%) were assessed with the final trained classification model to determine if they could be used to predict MSI status of cases failing microsatellite testing. The model identified one case as potentially MSI-H (FIG. 10), which failed testing due to a low number of total microsatellite sites assessed (n=23). Out of these 23 sites, 19 were determined as unstable by NGS sequencing. Per the archival pathology report, this tumor was deficient in MLH1 and PMS2 by IHC.

### b) Genomic Profiling

DNA and RNA were co-extracted from FFPE tissue specimens and submitted for library preparation, DNA sequencing, and RNA sequencing using OmniSeq® INSIGHT (OmniSeq, Buffalo, NY, USA). OmniSeq® INSIGHT is a next generation sequencing-based, laboratory-developed test for the detection of genomic variants, signatures, and immune gene expression in FFPE tumor tissue, performed in a laboratory accredited by the College of American Pathologists (CAP) and certified by the Clinical Laboratory Improvement Amendments (CLIA). DNA sequencing with the hybrid-capture-based TruSight® Oncology 500 assay (Illumina, San Diego, CA, USA) was used to detect small variants in the full exonic coding region of 523 genes (single and multi-nucleotide substitutions, insertions, and deletions), copy number alterations in 59 genes (gains and losses), MSI and TMB genomic signatures.

Briefly, for TCGA cases, tumor and normal specimens were taken from patients and processed in two resource centers. Then, aliquots of purified nucleic acids were shipped to genome sequencing centers where DNA sequencing of coding regions and profiling of genomic alterations was performed by whole-exome capture followed by sequencing on the SOLID or Illumina HiSeq platforms. Presence or absence of single nucleotide variants (SNVs) summarized at the gene-level were utilized as genomic features in the present work.

### c) Immune Gene Expression Profiling and Bioinformatics Processing

Gene expression of 395 immune-related genes was interrogated via an amplicon-based targeted RNA sequencing assay (Oncomine™ Immune Response Research Assay, ThermoFisher, Waltham, MA, USA). Absolute read counts for each gene transcript were generated using the Ion Torrent Suite Software plugin immuneResponseRNA (ThermoFisher, Waltham, MA), then background read counts from a sequenced no template control sample were subtracted to produce background subtracted read counts (BSRC). Per sample normalized reads per million (nRPM) values were then calculated to make RNA-seq measurements across runs and samples comparable. Briefly, sample BSRCs are normalized by obtaining sample-to-control ratios of 10 housekeeping gene BSRCs compared against a pre-constructed housekeeping gene RPM profile from an external, validated control sample, and the median ratio is used as a normalization ratio for the sample. Then, for each transcript, sample BSRCs are divided by the normalization ratio to obtain nRPM values. From the nRPM values, normalized gene expression ranks are calculated as a percentile rank from 0 to 100 by comparing nRPM values to those of a pan-cancer reference population derived from 735 unique tumors. FIG. 11 illustrates a summary of this workflow. The process was the same for TCGA COAD/READ cases except gene expression data was generated using whole-transcriptome RNA sequencing.

### d) Training and Testing of Machine Learning Classification Models

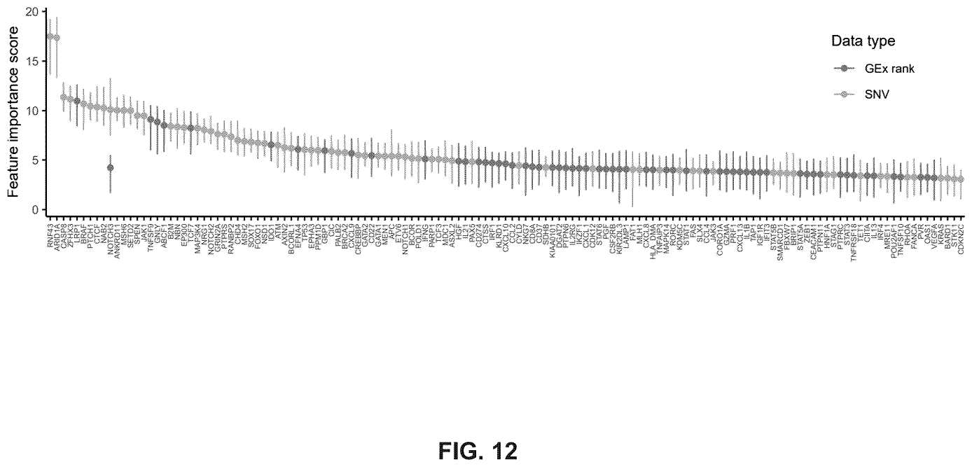

Using the CRC training dataset (see Table 1), feature selection was performed using the Boruta algorithm via the ‘Boruta’ function in the Boruta R package (e.g., v8.0.0) to determine what immune gene expression and SNVs were most important in differentiating MSI cases from microsatellite stable (MSS) cases. Feature selection was performed separately for gene expression and SNVs, and features confirmed by the Boruta algorithm (N=138 features) were extracted (along with TMB). FIG. 12 shows the identified features from the feature selection process. Sixty-two (62) gene expression changes, 76 genomic alterations (single nucleotide variants, insertions, and deletions), and TMB were identified as informative features for MSI prediction. Accordingly, these features were used as input for training 8 different machine learning models, commonly used for classification, for predicting MSI status.

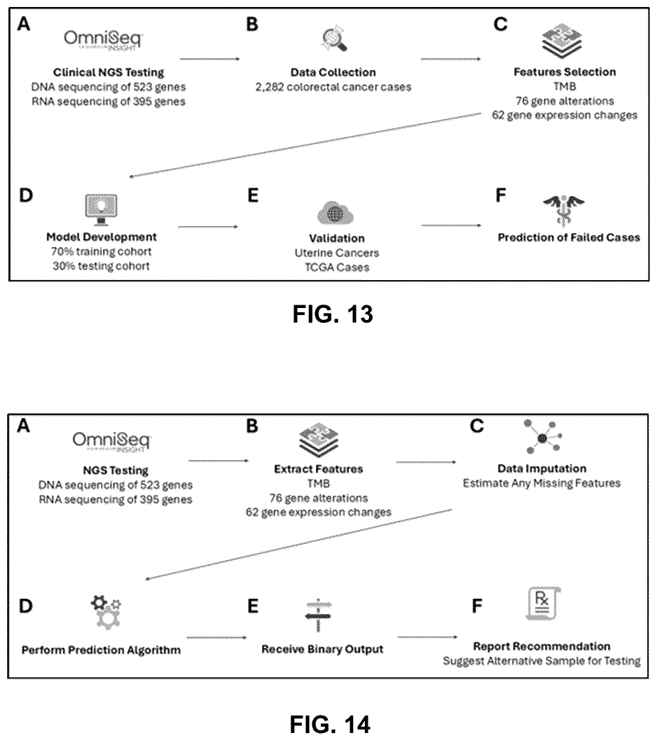

Models included generalized linear models (GLM), k-nearest neighbor (K-NN), linear discriminant analysis (LDA), support vector machine with linear kernel (SVM Linear), random forest (RF), classification and regression trees (CARTs) as implemented in the rpart R package, stochastic gradient boosting (GBM), and XGBoost with decision trees (XGBTree). Training of models was performed using the ‘train’ function in the caret R package (e.g., v6.0.94) with 10-fold cross validation. Trained models were then used to predict MSI status of cases in the CRC test dataset using the ‘predict’ function in caret. Model predictions of test cases were evaluated against their MSI status obtained from microsatellite testing using the ‘confusionMatrix’ function in caret R package, and common model evaluation metrics were compared across model types including accuracy, specificity, positive predictive value (PPV), negative predictive value (NPV), recall/sensitivity, F1, and balanced accuracy. Model predictions and evaluations were repeated for the TCGA COAD/READ cases, and models with the highest performances in both CRC and TCGA COAD/READ cohorts were evaluated in the UC cohort. Receiver operating curves (ROC) and precision-recall (PR) curves were generated, and area under the curve (AUC) calculated, for each cohort using the top performing model. AUC-ROC and -PR calculations were performed using the ‘evalm’ function from the MLeval R package (e.g., v0.3). The top performing model across all cohorts was then used to predict MSI status for CRC cases that previously failed microsatellite testing to assess the feasibility of using the model in a real-world setting to obtain MSI status for cases that may fail microsatellite testing. An overview of the training, testing, and model performance evaluation workflow can be found in FIG. 13.

### e) Predicting MSI Status for Cases Failing Microsatellite Testing

To predict MSI status for cases failing microsatellite testing using the trained classification model, data for model features (TMB, gene expression rank for 62 genes, gene-level summarized SNV presence or absence for 76 genes) are first extracted for failing cases. Then, data for failed cases are combined with passing cases (N=2,282) and any missing data imputed using the following steps:
1. Missing data for immune gene expression and gene-level summarized SNV presence or absence is imputed using the Multivariate Imputation by Chained Equations (MICE) method implemented in the mice R package (e.g., v3.16.0 used for the current study) with method set to predictive mean matching (PMM) and number of imputations and iterations set to 5.
2. Immune gene expression and gene-level summarized SNV presence or absence is used to fit a linear regression model for TMB using the ‘Im’ function in R with Formula 1:

log(TMB+1)~GEX_{1,2,3 . . . n}+SNV_{1,2,3 . . . n} Formula 1:
where GEX_{1, 2, 3 . . . n} and SNV_{1, 2, 3 . . . n} refer to the gene expression rank or gene-level summarized SNV presence or absence of each model feature, respectively.
3. The fitted linear regression model is used to predict missing TMB values by first generating predicted log (TMB) values using the ‘predict’ function in R with data for cases missing TMB, then taking the exponent of predicted values to obtain predicted TMB values.

Failed cases are combined with passing cases prior to imputation to increase the accuracy and robustness of the data imputation as cases failing microsatellite testing frequently have other missing genomic data such as SNV data and TMB. Once missing data has been imputed, MSI status for failed cases is predicted using the trained classification model and a classification of “likely MSI-high” or “likely MSS” is received based on the model's classification. The classification output would appear on the clinical report along with language that would suggest further testing for MSI. FIG. 14 shows an overview of the workflow for predicting MSI status in cases failing microsatellite testing.

### f) Results

Feature selection identified 138 features total including gene expression rank data for 62 genes and gene-level summarized SNV presence or absence for 76 genes (FIG. 12). Using these features, along with TMB, 8 different classification models were trained, tested, and evaluated on CRC case data resulting in performance metric values ranging from 0.58 (knn, recall/sensitivity) to 1 (rf and rpart, NPV and recall/sensitivity) (FIG. 15 and Tables 2 and 3).

TABLE 2
CRC testing

| Metric | glm | knn | Ida | svmLinear | rf | rpart | gbm | xgbTree |
|---|---|---|---|---|---|---|---|---|
| Accuracy | 0.965 | 0.95 | 0.972 | 0.98 | 0.99 | 0.987 | 0.993 | 0.99 |
| Specificity | 0.99 | 0.987 | 0.995 | 0.994 | 0.989 | 0.986 | 0.994 | 0.992 |
| PPV | 0.88 | 0.818 | 0.939 | 0.929 | 0.899 | 0.873 | 0.938 | 0.923 |
| NPV | 0.972 | 0.959 | 0.975 | 0.984 | 1 | 1 | 0.998 | 0.997 |
| Recall | 0.71 | 0.581 | 0.742 | 0.839 | 1 | 1 | 0.984 | 0.968 |
| F1 | 0.786 | 0.679 | 0.829 | 0.881 | 0.947 | 0.932 | 0.961 | 0.945 |
| Balanced Accuracy | 0.85 | 0.784 | 0.869 | 0.916 | 0.994 | 0.993 | 0.989 | 0.98 |

TABLE 3
Confusion matrices of top performing models

Reference

| | Prediction | MSS | MSI_H |
|---|---|---|---|
| RF | MSS | 616 | 0 |
| | MSI_H | 7 | 62 |
| RPART | MSS | 614 | 0 |
| | MSI_H | 9 | 62 |
| GBM | MSS | 619 | 1 |
| | MSI_H | 4 | 61 |

Abbreviations: RF: random forest; RPART: recursive partitioning and regressing trees; GBM: gradient boosting model; MSS: microsatellite stable; MSI_H: high microsatellite instability.

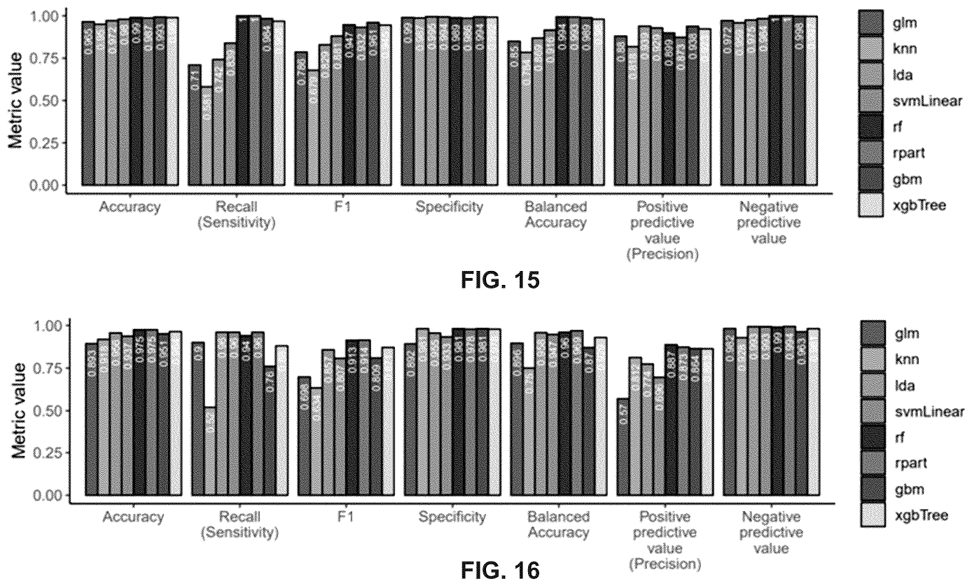

Both rf and rpart models had perfect recall/sensitivity, correctly classifying all MSI high cases, while gbm had the second highest missing only 1 MSI high case. When evaluating model performance within the TCGA COAD/READ cohort, again, rf, rpart, and gbm models were among the higher performers with the rpart model having the highest NPV (0.94), recall/sensitivity (0.96), F1 (0.91), and balanced accuracy (0.97) (FIG. 16 and Tables 4 and 5).

TABLE 4
TCGA COAD and READ testing

| Metric | glm | knn | Ida | svmLinear | rf | rpart | gbm | xgbTree |
|---|---|---|---|---|---|---|---|---|
| Accuracy | 0.893 | 0.918 | 0.956 | 0.937 | 0.975 | 0.975 | 0.951 | 0.964 |
| Specificity | 0.892 | 0.981 | 0.955 | 0.933 | 0.981 | 0.978 | 0.981 | 0.978 |
| PPV | 0.57 | 0.812 | 0.774 | 0.696 | 0.887 | 0.873 | 0.864 | 0.863 |
| NPV | 0.982 | 0.928 | 0.993 | 0.993 | 0.99 | 0.994 | 0.963 | 0.981 |
| Recall | 0.9 | 0.52 | 0.96 | 0.96 | 0.94 | 0.96 | 0.76 | 0.88 |
| F1 | 0.698 | 0.634 | 0.857 | 0.807 | 0.913 | 0.914 | 0.809 | 0.871 |
| Balanced Accuracy | 0.896 | 0.75 | 0.958 | 0.947 | 0.96 | 0.969 | 0.87 | 0.929 |

TABLE 5
Confusion matrices of top performing models

Reference

| | Prediction | MSS | MSI_H |
|---|---|---|---|
| RF | MSS | 308 | 3 |
| | MSI_H | 6 | 47 |
| RPART | MSS | 307 | 2 |
| | MSI_H | 7 | 48 |
| GBM | MSS | 308 | 12 |
| | MSI_H | 6 | 38 |

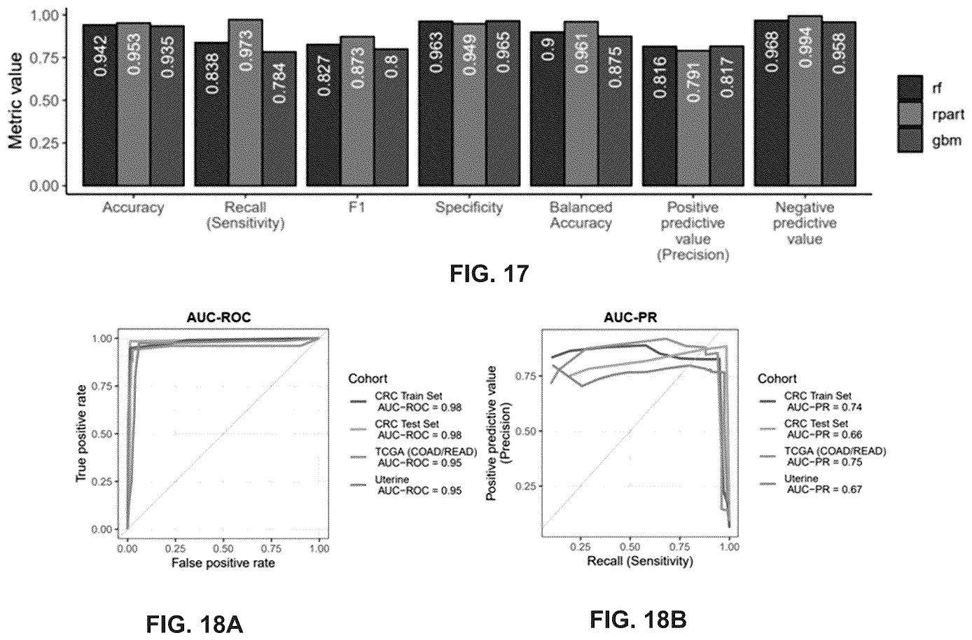

High performance of these models was observed when extended to UC cases with the rpart model having higher performance metrics than rf and gbm models except for specificity and PPV (FIG. 17 and Tables 6 and 7).

TABLE 6
UC testing

| Metric | rf | rpart | gbm |
|---|---|---|---|
| Accuracy | 0.942 | 0.953 | 0.935 |
| Specificity | 0.963 | 0.949 | 0.965 |
| PPV | 0.816 | 0.791 | 0.817 |
| NPV | 0.968 | 0.994 | 0.958 |
| Recall | 0.838 | 0.973 | 0.784 |
| F1 | 0.827 | 0.873 | 0.8 |
| Balanced Accuracy | 0.9 | 0.961 | 0.875 |

TABLE 7
Confusion matrices of top performing models

Reference

| | Prediction | MSS | MSI_H |
|---|---|---|---|
| RF | MSS | 361 | 12 |
| | MSI_H | 14 | 62 |
| GBM | MSS | 362 | 16 |
| | MSI_H | 13 | 58 |

Due to its high performance across cohorts (FIGS. 18A and 18B), especially for recall/sensitivity (see Table 8), the rpart model was chosen to use as the model for predicting MSI status of cases failing microsatellite testing. Using this model, 16 cases out of 187 total failed cases (8%) were predicted to be “likely MSI-high,” which is a frequency in line with what was observed in CRC cases that passed microsatellite testing (Table 1). While the majority of the 16 “likely MSI-high” cases did not have any available data from microsatellite testing (69%), 3 out of 5 cases (60%) that had some available data from testing showed evidence of MSI based on the limited microsatellite sites that were able to be assessed.

TABLE 8
Cohort demographics and diagnostic metrics comparing ML and direct
sequencing assessment of MSI status.

| | | | | MSI- | | | | |
|---|---|---|---|---|---|---|---|---|
| | Sample | Median Age | Female | High | Sensitivity | Specificity | PPV | NPV |
| Cohort | Size (n) | (range; y) | (%) | (%) | (%) | (%) | (%) | (%) |
| Training | 1597 | 65 (24 to ≥ 90) | 45% | 6% | NA | NA | NA | NA |
| Validation | 685 | 65 (29 to ≥ 90) | 46% | 9% | 100% | 99% | 87% | 100% |
| TCGA | 364 | 66 (31 to ≥ 90) | 47% | 14% | 96% | 98% | 87% | 99% |
| (COAD/READ) | | | | | | | | |
| Uterine | 449 | 69 (26 to ≥ 90) | 100% | 16% | 97% | 95% | 79% | 99% |
| Indeterminate | 77 | 65 (40 to 85) | 52% | NA | NA | NA | NA | NA |

### g) Discussion and Conclusions

MSI status is a critical biomarker in oncology for guiding the use of immunotherapies, such as PD-L1 inhibitors, which have shown robust and durable responses in patients with MSI-high tumors. Accurate determination of MSI status is essential, but technical limitations in NGS testing can sometimes result in failed determinations. Predicting MSI status in these cases ensures that patients who may benefit from immunotherapy are identified and appropriately treated. The algorithm addresses this by predicting MSI status from comprehensive NGS data, thus bridging the gap caused by failed tests and optimizing patient care. By integrating TMB, genomic alterations, and gene expression data, the algorithm provides an orthogonal approach to predict MSI status that is independent of direct sequencing of microsatellite sites. This method can identify cases that require further MSI testing, thereby ensuring that all patients receive comprehensive evaluations. The development of a prediction algorithm for MSI status is potentially beneficial to clinicians. The algorithm performs calculations on 139 datapoints and delivers a result within seconds, a task that would be infeasible for humans to complete manually in a clinically relevant timeframe. This algorithm was robustly identified among other commonly used algorithms, and in conjunction with our novel workflow for imputing missing datapoints in cases failing microsatellite testing, improves upon existing workflows for accurate identification of MSI-high cases.

### IX. ADDITIONAL CONSIDERATIONS

Specific details are given in the above description to provide a thorough understanding of the embodiments. However, it is understood that the embodiments can be practiced without these specific details. For example, circuits can be shown in block diagrams in order not to obscure the embodiments in unnecessary detail. In other instances, well-known circuits, processes, algorithms, structures, and techniques can be shown without unnecessary detail in order to avoid obscuring the embodiments.

Implementation of the techniques, blocks, steps and means described above can be done in various ways. For example, these techniques, blocks, steps and means can be implemented in hardware, software, or a combination thereof. For a hardware implementation, the processing units can be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described above, and/or a combination thereof.

Also, it is noted that the embodiments can be described as a process which is depicted as a flowchart, a flow diagram, a data flow diagram, a structure diagram, or a block diagram. Although a flowchart can describe the operations as a sequential process, many of the operations can be performed in parallel or concurrently. In addition, the order of the operations can be re-arranged. A process is terminated when its operations are completed, but could have additional steps not included in the figure. A process can correspond to a method, a function, a procedure, a subroutine, a subprogram, etc. When a process corresponds to a function, its termination corresponds to a return of the function to the calling function or the main function.

Furthermore, embodiments can be implemented by hardware, software, scripting languages, firmware, middleware, microcode, hardware description languages, and/or any combination thereof. When implemented in software, firmware, middleware, scripting language, and/or microcode, the program code or code segments to perform the tasks can be stored in a machine readable medium such as a storage medium. A code segment or machine-executable instruction can represent a procedure, a function, a subprogram, a program, a routine, a subroutine, a module, a software package, a script, a class, or any combination of instructions, data structures, and/or program statements. A code segment can be coupled to another code segment or a hardware circuit by passing and/or receiving information, data, arguments, parameters, and/or memory contents. Information, arguments, parameters, data, etc. can be passed, forwarded, or transmitted via any suitable means including memory sharing, message passing, ticket passing, network transmission, etc.

For a firmware and/or software implementation, the methodologies can be implemented with modules (e.g., procedures, functions, and so on) that perform the functions described herein. Any machine-readable medium tangibly embodying instructions can be used in implementing the methodologies described herein. For example, software codes can be stored in a memory. Memory can be implemented within the processor or external to the processor. As used herein the term “memory” refers to any type of long term, short term, volatile, nonvolatile, or other storage medium and is not to be limited to any particular type of memory or number of memories, or type of media upon which memory is stored.

Moreover, as disclosed herein, the term “storage medium”, “storage” or “memory” can represent one or more memories for storing data, including read only memory (ROM), random access memory (RAM), magnetic RAM, core memory, magnetic disk storage mediums, optical storage mediums, flash memory devices and/or other machine readable mediums for storing information. The term “machine-readable medium” includes, but is not limited to portable or fixed storage devices, optical storage devices, wireless channels, and/or various other storage mediums capable of storing that contain or carry instruction(s) and/or data.

While the principles of the disclosure have been described above in connection with specific apparatuses and methods, it is to be clearly understood that this description is made only by way of example and not as limitation on the scope of the disclosure.

What is claimed is:

1. A computer-implemented method, comprising:
performing a genomic instability testing on a first sample obtained from a subject to generate an indication of a genomic instability of the first sample, wherein the indication comprises a presence of the genomic instability, an absence of the genomic instability, or a failure to make the indication;
performing an AI-based genomic instability determination by:
obtaining, using one or more multi-analyte assays, multi-omics data for the subject, wherein the one or more multi-analyte assays comprise DNA sequencing and RNA sequencing, and wherein the multi-omics data comprises genomic alteration data for a first set of genes obtained using the DNA sequencing and expression data for a second set of immune genes obtained using the RNA sequencing;
inputting the multi-omics data into a machine learning model, wherein the machine learning model comprises a tree-based architecture configured to analyze one or more features by traversing a path from a root node to a terminal node in each tree of the tree-based architecture based on values of one or more features generated from the multi-omics data; and
predicting a genomic instability status of the subject using the machine learning model based on the paths and/or the terminal nodes; and
providing the predicted genomic instability status via a notification to a user interface or through a testing report.

2. The computer-implemented method of claim 1, further comprising:
in response to the failure to make the indication, providing the indication through the testing report; and
in response to the indication of the presence or absence of the genomic instability, comparing the indication with the predicted genomic instability status, and providing the comparison through the testing report confirming if the indication is consistent or inconsistent with the predicted genomic instability status.

3. The computer-implemented method of claim 1, wherein (i) the genomic instability is microsatellite instability (MSI), and the predicted genomic instability status is an MSI status, or (ii) the genomic instability is homologous recombination deficiency (HRD), and the predicted genomic instability status is an HRD status.

4. The computer-implemented method of claim 3, wherein (i) the MSI status is MSI-high or microsatellite stable (MSS), or (ii) the HRD status is HRD-high or HRD-low.

5. The computer-implemented method of claim 1, wherein the multi-omics data is obtained by:
obtaining a biopsy sample from the subject, wherein the biopsy sample is the first sample or a portion thereof, or a different sample;
extracting DNA and RNA from the biopsy sample;
performing the DNA sequencing to obtain DNA sequencing data;
performing the RNA sequencing to obtain RNA sequencing data;
generating the genomic alteration data for the first set of genes based on the DNA sequencing data; and
generating the expression data for the second set of immune genes based on the RNA sequencing data.

6. The computer-implemented method of claim 5, further comprising imputing missing genomic alteration data for one or more genes in the first set of genes or missing expression data for one or more immune genes in the second set of immune genes based on the DNA sequencing data, the RNA sequencing data, or available genomic alteration data or available expression data from one or more other genes in the rest of the first set of genes or the second set of immune genes.

7. The computer-implemented method of claim 1, further comprising performing a confirmatory testing using a second sample obtained from a subject to confirm the predicted genomic instability status, wherein the second sample is the first sample or a portion thereof, or a different sample.

8. The computer-implemented method of claim 7, wherein the confirmatory testing comprises one or more of:
(a) MLH1 promoter methylation analysis,
(b) mismatch repair gene sequencing,
(c) loss of heterozygosity (LOH) analysis,
(d) functional DNA repair assay,
(e) fluorescence in situ hybridization (FISH) assay,
(f) chromosomal microarray analysis,
(g) targeted sequencing using an extended genomic instability panel,
(h) Sanger sequencing, and
(i) immunohistochemistry for mismatch repair proteins.

9. The computer-implemented method of claim 1, further comprising outputting the testing report, wherein the testing report comprises the indication for the genomic instability testing, the predicted genomic instability status using the AI-based genomic instability determination, the multi-omics data and/or features used by the machine learning model to determine the predicted genomic instability status.

10. The computer-implemented method of claim 1, wherein the genomic alteration data comprises data of single nucleotide variants (SNVs), insertions and deletions (indels), copy number variations (CNVs), gene fusions, splice variants, and/or tumor mutational burden (TMB), and wherein the multi-omics data for the subject further comprises immunohistochemistry data, cell proliferation data, tumor inflammation data, and/or cancer testis antigen burden data.

11. The computer-implemented method of claim 10, wherein (i) the immunohistochemistry data comprises PD-L1 immunohistochemistry data, (ii) the cell proliferation data comprises Ki-67 proliferation index data, (iii) the tumor inflammation data comprises gene expression signatures associated with tumor inflammation, and/or (iv) the cancer testis antigen burden data comprises expression levels of one or more cancer testis antigens.

12. The computer-implemented method of claim 10, further comprising:
(i) staining a formalin-fixed, paraffin-embedded (FFPE) tissue sample obtained from the subject with an antibody specific to a protein of interest and evaluating the stained FFPE tissue sample by light microscopy or digital image analysis to obtain the immunohistochemistry data, the cell proliferation data, the tumor inflammation data, and/or the cancer testis antigen burden data; and/or
(ii) extracting RNA from the FFPE tissue sample and performing gene expression profiling RNA sequencing or quantitative PCR to obtain the cell proliferation data, the tumor inflammation data, and/or the cancer testis antigen burden data.

13. The computer-implemented method of claim 1, wherein the machine learning model is trained using training samples with known genomic instability statuses to learn patterns from multi-omics data of the training samples to predict genomic instability statuses of training samples.

14. The computer-implemented method of claim 13, further comprising training the machine learning model, wherein the training comprises:
obtaining training data associated with the training samples, wherein the training data comprises genomic alteration data, gene expression data, and the known genomic instability statuses;
selecting, using a feature-selection model, candidate features from a set of features based on the training data, wherein the candidate features are predictors to the genomic instability status;
training the machine learning model using input data with the candidate features to predict the genomic instability status; and
outputting the trained machine learning model.

15. The computer-implemented method of claim 14, further comprising
dividing the input data into training sets and testing sets;
performing the training using the training sets to learn a mapping from the candidate features to the genomic instability status;
evaluating the trained machine learning model on the testing sets to generate one or more performance metrics indicative of an ability of the trained machine learning model to predict the genomic instability status; and
outputting the trained machine learning model with the one or more performance metrics.

16. The computer-implemented method of claim 14, wherein the training samples are biological samples obtained from patients having been diagnosed with cancer, and wherein the cancer is one or more cancer selected from the group consisting of: colorectal cancer, colon adenocarcinoma, rectum adenocarcinoma, uterine cancer, adrenal gland cancer, bile duct cancer, bladder cancer, bone cancer, bone marrow and blood cancer, brain cancer, breast cancer, cervix cancer, esophageal cancer, eye cancer, head and neck cancer, kidney cancer, liver cancer, lung cancer, lymph node cancer, nervous system cancer, ovarian cancer, pancreatic cancer, pleura cancer, prostate cancer, skin cancer, soft tissue cancer, stomach cancer, testicular cancer, thymus cancer, thyroid cancer, and uterine cancer.

17. The computer-implemented method of claim 14, further comprising:
selecting a plurality of machine learning algorithms;
training each machine learning model with one or more machine learning algorithm in the plurality of machine learning algorithms;
generating a set of performance metrics for each trained machine learning model; and
selecting the machine learning model based on a set of performance metrices.

18. The computer-implemented method of claim 17, wherein the feature-selection model is a Boruta algorithm-based model or a random forest model, and wherein the plurality of machine learning algorithms comprises Classification and Regression Trees (CART), stochastic gradient boosting, Gradient Boosting Machine (GBM), K-Nearest Neighbors (K-NN), Linear Discriminant Analysis (LDA), Logistic Regression, Multi-Layer Perceptron (MLP), Naive Bayes, Random Forest, Support Vector Machine with Radial Basis Function Kernel (SVM Radial), and/or Extreme Gradient Boosting (XGBoost).

19. A non-transitory computer-readable medium storing instructions which, when executed by one or more processors, cause the one or more processors to perform operations comprising:
performing a genomic instability testing on a first sample obtained from a subject to generate an indication of a genomic instability of the first sample, wherein the indication comprises a presence of the genomic instability, an absence of the genomic instability, or a failure to make the indication;
performing an AI-based genomic instability determination by:
obtaining, using one or more multi-analyte assays, multi-omics data for the subject, wherein the one or more multi-analyte assays comprise DNA sequencing and RNA sequencing, and wherein the multi-omics data comprises genomic alteration data for a first set of genes obtained using the DNA sequencing and expression data for a second set of immune genes obtained using the RNA sequencing;
inputting the multi-omics data into a machine learning model, wherein the machine learning model comprises a tree-based architecture configured to analyze one or more features by traversing a path from a root node to a terminal node in each tree of the tree-based architecture based on values of one or more features generated from the multi-omics data; and
predicting a genomic instability status of the subject using the machine learning model based on the paths and/or the terminal nodes; and
providing the predicted genomic instability status via a notification to a user interface or through a testing report.

20. A system comprising:
one or more processors; and
one or more computer-readable media storing instructions which, when executed by the one or more processors, cause the system to perform the operations comprising:
performing a genomic instability testing on a first sample obtained from a subject to generate an indication of a genomic instability of the first sample, wherein the indication comprises a presence of the genomic instability, an absence of the genomic instability, or a failure to make the indication;
performing an AI-based genomic instability determination by:
obtaining, using one or more multi-analyte assays, multi-omics data for the subject, wherein the one or more multi-analyte assays comprise DNA sequencing and RNA sequencing, and wherein the multi-omics data comprises genomic alteration data for a first set of genes obtained using the DNA sequencing and expression data for a second set of immune genes obtained using the RNA sequencing;
inputting the multi-omics data into a machine learning model, wherein the machine learning model comprises a tree-based architecture configured to analyze one or more features by traversing a path from a root node to a terminal node in each tree of the tree-based architecture based on values of one or more features generated from the multi-omics data; and
predicting a genomic instability status of the subject using the machine learning model based on the paths and/or the terminal nodes; and
providing the predicted genomic instability status via a notification to a user interface or through a testing report.
